(19) United States
(12) Patent Application Publication
(10) Pub. No.: US 2020/0190087 A1
(43) Pub. Date: Jun. 18, 2020

(54) 2-PHENYL-2H-PYRAZOLO[3,4-D]PYRIDAZINE DERIVATIVES HAVING ACTIVITY AGAINST PAIN

(71) Applicant: ESTEVE PHARMACEUTICALS, S.A., Barcelona (ES)

(72) Inventors: Félix CUEVAS-CORDOBÉS, Valdemoro (ES); Carmen ALMANSA-ROSALES, Barcelona (ES)

(21) Appl. No.: 16/464,339

(22) PCT Filed: Nov. 30, 2017
(86) PCT No.: PCT/EP2017/080948

§ 371 Date: May 28, 2019

(30) Foreign Application Priority Data
Nov. 30, 2016 (EP) 16382576.3

(51) Int. Cl. C07D 487/04

(52) U.S. Cl. CPC C07D 487/04

(57) ABSTRACT

The present invention relates to 2-phenyl-2H-pyrazolo[3,4-d]pyridazine derivatives having pharmacological activity towards the α2δ subunit, in particular the α2δ-1 subunit, of the voltage-gated calcium channel, in particular having dual pharmacological activity towards both the α2δ subunit, in particular the α2δ-1 subunit, of the voltage-gated calcium channel and the μ-opioid receptor. The present invention also relates to processes of preparation of such compounds, to pharmaceutical compositions comprising them, and to their use in therapy, in particular for the treatment of pain.

### FIELD OF THE INVENTION

The present invention relates to compounds having pharmacological activity towards the α₂δ subunit of the voltage-gated calcium channel. In particular, the present invention relates to compounds having dual pharmacological activity towards both the α₂δ subunit of the voltage-gated calcium channel, and the μ-opioid receptor (MOR or mu-opioid receptor). More particularly, the present invention relates to 2-phenyl-2H-pyrazolo[3,4-d]pyridazine derivatives having this pharmacological activity, to processes of preparation of such compounds, to pharmaceutical compositions comprising them, and to their use in therapy, in particular for the treatment of pain.

### BACKGROUND OF THE INVENTION

The adequate management of pain constitutes an important challenge, since currently available treatments provide in many cases only modest improvements, leaving many patients unrelieved (Turk, D. C., Wilson, H. D., Cahana, A.; 2011; Lancet; 377; 2226-2235). Pain affects a big portion of the population with an estimated prevalence of 20% and its incidence, particularly in the case of chronic pain, is increasing due to the population ageing. Additionally, pain is clearly related to comorbidities, such as depression, anxiety and insomnia, which lead to important productivity losses and socio-economical burden (Goldberg, D. S., McGee, S. J.; 2011; BMC Public Health; 11; 770). Existing pain therapies include non-steroidal anti-inflammatory drugs (NSAIDs), opioid agonists, calcium channel blockers and antidepressants, but they are much less than optimal regarding their safety ratio. All of them show limited efficacy and a range of secondary effects that preclude their use, especially in chronic settings.

Voltage-gated calcium channels (VGCC) are required for many key functions in the body. Different subtypes of voltage-gated calcium channels have been described (Zamponi et al., Pharmacol Rev. 2015 67:821-70). The VGCC are assembled through interactions of different subunits, namely α₁ (Caᵥa₁), β (Caᵥβ) α₂δ (Caᵥα₂δ) and γ (Caᵥγ). The α₁ subunits are the key porous forming units of the channel complex, being responsible for the Ca²⁺ conduction and generation of Ca²⁺ influx. The α₂δ, β, and γ subunits are auxiliary, although very important for the regulation of the channel since they increase the expression of the α₁ subunits in the plasma membrane as well as modulate their function, resulting in functional diversity in different cell types. Based on their physiological and pharmacological properties, VGCC can be subdivided into low voltage-activated T-type (Caᵥ3.1, Caᵥ3.2, and Caᵥ3.3), and high voltage-activated L-(Caᵥ1.1 through Caᵥ1.4), N-(Caᵥ2.2), P/Q-(Caᵥ2.1), and R-(Caᵥ2.3) types, depending on the channel forming Caᵥα subunits. All of these five subclasses are found in the central and peripheral nervous systems. Regulation of intracellular calcium through activation of these VGCC plays obligatory roles in: 1) neurotransmitter release, 2) membrane depolarization and hyperpolarization, 3) enzyme activation and inactivation, and 4) gene regulation (Perret and Luo, Neurotherapeutics. 2009 6:679-92; Zamponi et al., 2015 supra; Neumaier et al., Prog Neurobiol. 2015 129:1-36.). A large body of data has clearly indicated that VGCC are implicated in mediating various disease states including pain processing. Drugs interacting with the different calcium channel subtypes and subunits have been developed. Current therapeutic agents include drugs targeting L-type Caᵥ1.2 calcium channels, particularly 1,4-dihydropyridines, which are widely used in the treatment of hypertension. T-type (Caᵥ3) channels are the target of ethosuximide, widely used in absence epilepsy. Ziconotide, a peptide blocker of N-type (Caᵥ2.2) calcium channels, has been approved as a treatment of intractable pain. (Perret and Luo, 2009, supra; Vink and Alewood, Br J Pharmacol. 2012 167:970-89.). The Caᵥ1 and Caᵥ2 subfamilies contain an auxiliary α₂δ subunit, which is the therapeutic target of the gabapentinoid drugs of value in certain epilepsies and chronic neuropathic pain. To date, there are four known α₂δ subunits, each encoded by a unique gene and all possessing splice variants. Each α₂δ protein is encoded by a single messenger RNA and is posttranslationally cleaved and then linked by disulfide bonds. Four genes encoding α₂δ subunits have now been cloned. α₂δ-1 was initially cloned from skeletal muscle and shows a fairly ubiquitous distribution. The α₂δ-2 and α₂δ-3 subunits were subsequently cloned from brain. The most recently identified subunit, α₂δ-4, is largely nonneuronal. The human α₂δ-4 protein sequence shares 30, 32 and 61% identity with the human α₂δ-1, α₂δ-2 and α₂δ-3 subunits, respectively. The gene structure of all α₂δ subunits is similar. All α₂δ subunits show several splice variants (Davies et al., Trends Pharmacol Sci. 2007 28:220-8.; Dolphin AC, Nat Rev Neurosci. 2012 13:542-55., Biochim Biophys Acta. 2013 1828:1541-9.).

The Caᵥα₂δ-1 subunit may play an important role in neuropathic pain development (Perret and Luo, 2009, supra; Vink and Alewood, 2012, supra). Biochemical data have indicated a significant Caᵥα₂δ-1, but not Caᵥα₂δ-2, subunit upregulation in the spinal dorsal horn, and DRG (dorsal root ganglia) after nerve injury that correlates with neuropathic pain development. In addition, blocking axonal transport of injury-induced DRG Caᵥα₂δ-1 subunit to the central presynaptic terminals diminishes tactile allodynia in nerve injured animals, suggesting that elevated DRG Caᵥα₂δ-1 subunit contributes to neuropathic allodynia.

The Caᵥα₂δ-1 subunit (and the Caᵥα₂δ-2, but not Caᵥα₂δ-3 and Caᵥα₂δ-4, subunits) is the binding site for gabapentin which has anti-allodynic/hyperalgesic properties in patients and animal models. Because injury-induced Caᵥα₂δ-1 expression correlates with neuropathic pain development and maintenance, and various calcium channels are known to contribute to spinal synaptic neurotransmission and DRG neuron excitability, injury-induced Caᵥα₂δ-1 subunit upregulation may contribute to the initiation and maintenance of neuropathic pain by altering the properties and/or distribution of VGCC in the subpopulation of DRG neurons and their central terminals, therefore modulating excitability and/or synaptic neuroplasticity in the dorsal horn. Intrathecal antisense oligonucleotides against the Caᵥα₂δ-1 subunit can block nerve injury-induced Caᵥα₂δ-1 upregulation and prevent the onset of allodynia and reserve established allodynia.

As mentioned above, the α₂δ subunits of VGCC form the binding site for gabapentin and pregabalin, which are structural derivatives of the inhibitory neurotransmitter GABA although they do not bind to GABAA, GABAB, or benzodiazepine receptors, or alter GABA regulation in animal brain preparations. The binding of gabapentin and pregabalin to the Caᵥα₂δ subunit results in a reduction in the calcium-dependent release of multiple neurotransmitters, leading to efficacy and tolerability for neuropathic pain management. Gabapentinoids may also reduce excitability by inhibiting synaptogenesis (Perret and Luo, 2009, supra; Vink and Alewood, 2012, supra, Zamponi et al., 2015, supra).

Thus, the present invention relates to compounds with inhibitory effect towards the α₂δ subunit, in particular the α₂δ-1 subunit, of voltage-gated calcium channels.

As mentioned before, there are few available therapeutic classes for the treatment of pain, and opioids are among the most effective, especially when addressing severe pain states. They act through three different types of opioid receptors (mu, kappa and gamma) which are transmembrane G-protein coupled receptors (GPCRs). Still, the main analgesic action is attributed to the activation of the μ-opioid receptor (MOR). However, the general administration of MOR agonists is limited due to their important side effects, such as constipation, respiratory depression, tolerance, emesis and physical dependence [Meldrum, M. L. (Ed.). Opioids and Pain Relief: A Historical Perspective. Progress in Pain Research and Management, Vol 25. IASP Press, Seattle, 2003]. Additionally, MOR agonists are not optimal for the treatment of chronic pain as indicated by the diminished effectiveness of morphine against chronic pain conditions. This is especially proven for the chronic pain conditions of neuropathic or inflammatory origin, in comparison to its high potency against acute pain. The finding that chronic pain can lead to MOR down-regulation may offer a molecular basis for the relative lack of efficacy of morphine in long-term treatment settings [Dickenson, A. H., Suzuki, R. Opioids in neuropathic pain: Clues from animal studies. Eur J Pain 9, 113-6 (2005)]. Moreover, prolonged treatment with morphine may result in tolerance to its analgesic effects, most likely due to treatment-induced MOR down-regulation, internalization and other regulatory mechanisms. As a consequence, long-term treatment can result in substantial increases in dosing in order to maintain a clinically satisfactory pain relief, but the narrow therapeutic window of MOR agonists finally results in unacceptable side effects and poor patient compliance.

Polypharmacology is a phenomenon in which a drug binds multiple rather than a single target with significant affinity. The effect of polypharmacology on therapy can be positive (effective therapy) and/or negative (side effects). Positive and/or negative effects can be caused by binding to the same or different subsets of targets; binding to some targets may have no effect. Multi-component drugs or multi-targeting drugs can overcome toxicity and other side effects associated with high doses of single drugs by countering biological compensation, allowing reduced dosage of each compound or accessing context-specific multitarget mechanisms. Because multitarget mechanisms require their targets to be available for coordinated action, one would expect synergies to occur in a narrower range of cellular phenotypes given differential expression of the drug targets than would the activities of single agents. In fact, it has been experimentally demonstrated that synergistic drug combinations are generally more specific to particular cellular contexts than are single agent activities, such selectivity is achieved through differential expression of the drugs' targets in cell types associated with therapeutic, but not toxic, effects (Lehar et al., Nat Biotechnol 2009; 27: 659-666.).

In the case of chronic pain, which is a multifactorial disease, multi-targeting drugs may produce concerted pharmacological intervention of multiple targets and signaling pathways that drive pain. Because they actually make use of biological complexity, multi-targeting (or multi-component drugs) approaches are among the most promising avenues toward treating multifactorial diseases such as pain (Gilron et al., Lancet Neurol. 2013 November; 12(11):1084-95.). In fact, positive synergistic interaction for several compounds, including analgesics, has been described (Schröder et al., J Pharmacol Exp Ther. 2011; 337:312-20. Erratum in: J Pharmacol Exp Ther. 2012; 342:232.; Zhang et al., Cell Death Dis. 2014; 5:e1138.; Gilron et al., 2013, supra).

Given the significant differences in pharmacokinetics, metabolisms and bioavailability, reformulation of drug combinations (multi-component drugs) is challenging. Further, two drugs that are generally safe when dosed individually cannot be assumed to be safe in combination. In addition to the possibility of adverse drug-drug interactions, if the theory of network pharmacology indicates that an effect on phenotype may derive from hitting multiple targets, then that combined phenotypic perturbation may be efficacious or deleterious. The major challenge to both drug combination strategies is the regulatory requirement for each individual drug to be shown to be safe as an individual agent and in combination (Hopkins, Nat Chem Biol. 2008; 4:682-90.).

An alternative strategy for multitarget therapy is to design a single compound with selective polypharmacology (multi-targeting drug). It has been shown that many approved drugs act on multiple targets. Dosing with a single compound may have advantages over a drug combination in terms of equitable pharmacokinetics and biodistribution. Indeed, troughs in drug exposure due to incompatible pharmacokinetics between components of a combination therapy may create a low-dose window of opportunity where a reduced selection pressure can lead to drug resistance. In terms of drug registration, approval of a single compound acting on multiple targets faces significantly lower regulatory barriers than approval of a combination of new drugs (Hopkins, 2008, supra).

Thus, in a preferred embodiment, the compounds of the present invention, having inhibitory effects towards the α₂δ subunit, in particular the α₂δ-1 subunit, of voltage-gated calcium channels, additionally inhibit mu opioid receptor. The present invention relates also to the advantages of having dual activity, for μ-receptor and the α₂δ-1 subunit of voltage-gated calcium channels, in the same molecule to treat chronic pain.

In this way, the present invention relates to compounds having a mechanism of action on blocking the α₂δ subunit, in particular the α₂δ-1 subunit, of voltage-gated calcium channels. The present invention also relates to compounds having a complementary dual mechanism of action (μ-receptor agonist and blocker of the α₂δ subunit, in particular the α₂δ-1 subunit, of voltage-gated calcium channels) which implies a better profile of tolerability than the strong opioids (morphine, oxycodone, fentanyl etc) and/or better efficacy and tolerability than gabapentinoids (pregabalin and gabapentin).

Pain is multimodal in nature, since in nearly all pain states several mediators, signaling pathways and molecular mechanisms are implicated. Consequently, monomodal therapies can be complemented with a dual mechanism of action to provide complete pain relief. Currently, combining existing therapies is a common clinical practice and many efforts are directed to assess the best combination of available drugs in clinical studies (Mao, J., Gold, M. S., Backonja, M.; 2011; J. Pain; 12; 157-166).

Accordingly, there is still a need to find compounds that have an alternative or improved pharmacological activity in the treatment of pain, being both effective and showing the desired selectivity, and having good “drugability” properties, i.e. good pharmaceutical properties related to administration, distribution, metabolism and excretion.

The authors of the present invention, have found a serie of compounds that show pharmacological activity towards the α₂δ subunit, in particular the α₂δ-1 subunit, of the voltage-gated calcium channel, or compounds that show dual pharmacological activity towards both the α₂δ subunit, in particular the α₂δ-1 subunit, of the voltage-gated calcium channel and the μ-opioid receptor (MOR or mu-opioid receptor) resulting in an innovative, effective, complementary and alternative solution for the treatment of pain.

In view of the existing results of the currently available therapies and clinical practices, the present invention offers a solution by developing compounds binding to a single target or by combining in a single compound binding to two different targets relevant for the treatment of pain. This was mainly achieved by providing the compounds according to the invention that bind to the α₂δ subunit, in particular the α₂δ-1 subunit, of the voltage-gated calcium channel, or both to the μ-opioid receptor and to the α₂δ subunit, in particular the α₂δ-1 subunit, of the voltage-gated calcium channel.

### SUMMARY OF THE INVENTION

In this invention a family of structurally distinct 2-phenyl-2H-pyrazolo[3,4-d]pyridazine derivatives, encompassed by formula (I), which have a pharmacological activity towards the α₂δ subunit, in particular the α₂δ-1 subunit, of the voltage-gated calcium channel, or which have a dual pharmacological activity towards both the α₂δ subunit, in particular the α₂δ-1 subunit, of the voltage-gated calcium channel and the μ-opioid receptor, were identified thus solving the above problem of identifying alternative or improved pain treatments by offering such compounds.

The main object of the invention is directed to a compound having binding capacity to the α₂δ subunit, in particular the α₂δ-1 subunit, of the voltage-gated calcium channel for use in the treatment of pain.

Another object of the invention is directed to a compound having a dual activity for binding to the α₂δ subunit, in particular the α₂δ-1 subunit, of the voltage-gated calcium channel and the μ-opioid receptor for use in the treatment of pain.

As this invention is aimed at providing a compound or a chemically related series of compounds which act as ligands of the α₂δ subunit, in particular the α₂δ-1 subunit, of the voltage-gated calcium channel and/or the μ-opioid receptor it is a very preferred embodiment if the compound has a binding expressed as Kᵢ responding to the following scales:

Kᵢ(μ) is preferably<1000 nM, more preferably<500 nM, even more preferably<100 nM.

Kᵢ(α₂δ-1) is preferably<10000 nM, more preferably<5000 nM, even more preferably<3000 nM or even more preferably<500 nM.

The invention is directed in a main aspect to a compound of general Formula (In),

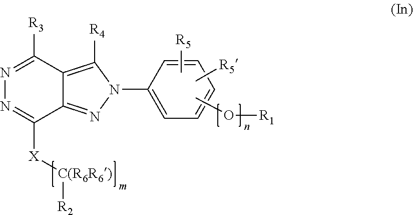

wherein R₁, R₂, R₃, R₄, R₅, R_{5′}, R₆, R_{6′}, X and m and n are as defined below in the detailed description.

The invention is directed in another aspect to a compound of general Formula (I),

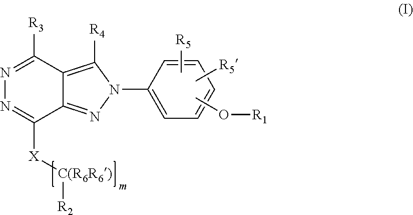

wherein R₁, R₂, R₃, R₄, R₅, R_{5′}, R₆, R_{6′}, X and m are as defined below in the detailed description.

A further object of the invention refers to the processes for preparation of compounds of general formula (In).

A still further object of the invention refers to the use of intermediate compounds for the preparation of a compound of general formula (In).

It is also an object of the invention a pharmaceutical composition comprising a compound of formula (In).

A further object of the invention refers to the processes for preparation of compounds of general formula (I).

A still further object of the invention refers to the use of intermediate compounds for the preparation of a compound of general formula (I).

It is also an object of the invention a pharmaceutical composition comprising a compound of formula (I).

Finally, it is an object of the invention the use of compound as a medicament and more particularly for the treatment of pain and pain related conditions.

### DETAILED DESCRIPTION OF THE INVENTION

The invention is directed to a family of structurally distinct 2-phenyl-2H-pyrazolo[3,4-d]pyridazine derivatives which have primary pharmacological activity towards the α₂δ subunit, in particular the α₂δ-1 subunit, of the voltage-gated calcium channel or which have a dual pharmacological activity towards both the α₂δ subunit, in particular the α₂δ-1 subunit, of the voltage-gated calcium channel and the μ-opioid receptor.

The invention is directed to compounds having primary activity binding to the α₂δ subunit, in particular the α₂δ-1 subunit, of the voltage-gated calcium channel or having a dual activity binding to the α₂δ subunit, in particular the α₂δ-1 subunit, of the voltage-gated calcium channel and the μ-opioid receptor for use in the treatment of pain.

As this invention is aimed at providing a compound or a chemically related series of compounds which act as ligands of the α_{2δ} subunit, in particular the α₂δ-1 subunit, of the voltage-gated calcium channel or as dual ligands of the α₂δ subunit, in particular the α₂δ-1 subunit, of the voltage-gated calcium channel and the μ-opioid receptor it is a preferred embodiment if the compound has a binding expressed as Kᵢ responding to the following scales:

Kᵢ(μ) is preferably<1000 nM, more preferably<500 nM.

Kᵢ(α₂δ-1) is preferably<10000 nM, more preferably<5000 nM, even more preferably<3000 nM or even more preferably<500 nM.

The applicant has surprisingly found that the problem of providing a new effective and alternative solution for treating pain and pain related disorders can be solved by using an analgesic approach using binding to the α₂δ subunit, in particular the α₂δ-1 subunit, of the voltage-gated calcium channel or a multimodal balanced analgesic approach combining two different synergistic activities in a single drug (i.e., dual ligands which are bifunctional and bind to μ-opioid receptor and to α₂δ subunit, in particular the α₂δ-1 subunit, of the voltage-gated calcium channel), thereby enhancing through the α₂δ blockade without increasing the undesirable side effects of the μ-opioid activity. This supports the therapeutic value of a dual agent, whereby the α₂δ binding component acts as an intrinsic adjuvant of the MOR binding component.

A dual compound that possess binding to both the μ-opioid receptor and to the α₂δ subunit of the voltage-gated calcium channel shows a highly valuable therapeutic potential by achieving an outstanding analgesia (enhanced in respect to the potency of the opioid component alone) with a reduced side-effect profile (safety margin increased compared to that of the opioid component alone) versus existing opioid therapies.

Advantageously, the compounds according to the present invention would in addition show one or more the following functionalities: blockade of the α₂δ subunit, in particular the α₂δ-1 subunit, of the voltage-gated calcium channel and μ-opioid receptor agonism.

It has to be noted, though, that functionalities “antagonism” and “agonism” are also sub-divided in their effect into subfunctionalities like partial agonism or inverse agonism. Accordingly, the functionalities of the compounds should be considered within a relatively broad bandwidth.

An antagonist blocks or dampens agonist-mediated responses. Known subfunctionalities are neutral antagonists or inverse agonists.

An agonist increases the activity of the receptor above its basal level. Known subfunctionalities are full agonists, or partial agonists.

In addition, the two mechanisms complement each other since MOR agonists are only marginally effective in the treatment of neuropathic pain, while the blockers of the α₂δ subunit, in particular the α₂δ-1 subunit, of voltage-gated calcium channels show outstanding effects in preclinical neuropathic pain models. Thus, the α₂δ component, in particular the α₂δ-1 component, adds unique analgesic actions in opioid-resistant pain. Finally, the dual approach has clear advantages over MOR agonists in the treatment of chronic pain as lower and better tolerated doses would be needed based on the potentiation of analgesia but not of the adverse events of MOR agonists.

A further advantage of using designed multiple ligands is a lower risk of drug-drug interactions compared to cocktails or multi-component drugs, thus involving simpler pharmacokinetics and less variability among patients. Additionally, this approach may improve patient compliance and broaden the therapeutic application in relation to monomechanistic drugs, by addressing more complex aetiologies. It is also seen as a way of improving the R&D output obtained using the “one drug-one target” approach, which has been questioned over the last years [Bornot A, Bauer U, Brown A, Firth M, Hellawell C, Engkvist O. Systematic Exploration of Dual-Acting Modulators from a Combined Medicinal Chemistry and Biology Perspective. J. Med. Chem, 56, 1197-1210 (2013)].

In its broader aspect, the present invention is directed to compounds of general Formula (In):

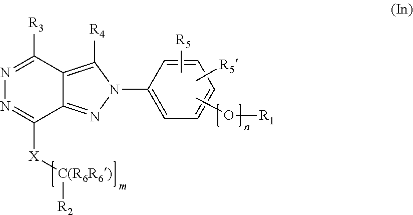

wherein

m is 0, 1, 2, 3, 4 or 5;

n is 0 or 1;

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl; wherein
Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;
R_{x′} is selected from hydrogen, substituted or unsubstituted alkylaryl, substituted or unsubstituted alkylcycloalkyl and substituted or unsubstituted alkylheterocyclyl;
wherein R₈ is selected from hydrogen, substituted or unsubstituted C₁₋₆ alkyl, substituted or unsubstituted C₂₋₆ alkenyl and substituted or unsubstituted C₂₋₆ alkynyl;

R₁ is selected from substituted or unsubstituted C₁₋₆ alkyl, substituted or unsubstituted C₂₋₆ alkenyl and substituted or unsubstituted C₂₋₆ alkynyl;

R₂ is selected from —NR₇R_{7′″}, —CN and substituted or unsubstituted N-containing-heterocyclyl;
wherein R₇ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc;
and wherein R_{7′″} is selected from hydrogen, substituted or unsubstituted C₁₋₆ alkyl, substituted or unsubstituted C₂₋₆ alkenyl and substituted or unsubstituted C₂₋₆ alkynyl;

R₃ is selected from hydrogen, substituted or unsubstituted C₁₋₆ alkyl, substituted or unsubstituted C₂₋₆ alkenyl and substituted or unsubstituted C₂₋₆ alkynyl;

R₄ is selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9″}, —SR₉ , —S(O)R₉, S(O)₂R₉, —CN, haloalkyl, haloalkoxy, —C(O)OR₉, —C(O)NR₉R_{9′}, —NR₉S(O)₂NR_{9′}R_{9″} and C(CH₃)₂OR₉;
wherein R₉, R_{9′} and R_{9″} are independently selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl; optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof;

In another aspect, the present invention is directed to compounds of general Formula (I):

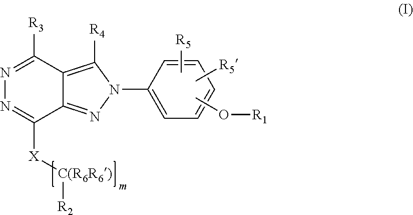

wherein

m is 0, 1, 2, 3, 4 or 5;

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl;
Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈—;
R_{x′}, is selected from hydrogen, substituted or unsubstituted alkylaryl, substituted or unsubstituted alkylcycloalkyl and substituted or unsubstituted alkylheterocyclyl;
wherein R₈ is selected from hydrogen, substituted or unsubstituted C₁₋₆ alkyl, substituted or unsubstituted C₂₋₆ alkenyl and substituted or unsubstituted C₂₋₆ alkynyl;

R₁ is selected from substituted or unsubstituted C₁₋₆ alkyl, substituted or unsubstituted C₂₋₆ alkenyl and substituted or unsubstituted C₂₋₆ alkynyl;

R₂ is selected from —NR₇R_{7′″}, —CN and substituted or unsubstituted N-containing-heterocyclyl;

R₃ is selected from hydrogen, substituted or unsubstituted C₁₋₆ alkyl, substituted or unsubstituted C₂₋₆ alkenyl and substituted or unsubstituted C₂₋₆ alkynyl;

R₄ is selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9″}, —SR₉ , —S(O)R₉, S(O)₂R₉, —CN, haloalkyl, haloalkoxy, —C(O)OR₉, —C(O)NR₉R_{9′}, —NR₉S(O)₂NR_{9′}R_{9″} and C(CH₃)₂OR₉;
wherein R₉, R_{9′} and R_{9″} are independently selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl;

These compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment, these compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof.

In a particular embodiment the following proviso applies:

the point of attachment of the —X-[CR₆R_{6′}]ₘ—R₂ moiety to the pyrazolopyridazine structure is not represented by a nitrogen atom

In a particular embodiment the following proviso applies:

—X—[CR₆R_{6′}]ₘ—R₂ is attached to the pyrazolopyridazine structure through a carbon atom.

In a particular embodiment the following proviso applies:

when X is a bond and m is 0, then R₂ is not —NR₇R_{7′″}.

In a particular embodiment the following proviso applies:

when X is a bond and m is 0 and R₂ is N-containing-heterocyclyl, then said N-containing-heterocyclyl is attached to the pyrazolopyridazine structure through a carbon atom.

In another particular embodiment the following proviso applies:

when R₂ is a N-containing heterocyclyl mono-substituted on the nitrogen with R₇ₐ, then R₇ₐ is selected from substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc.

These provisos apply to the compounds of the present invention, that is, to compounds of formula (I) or compounds of formula (In),(I), (I²′), (I³′), (I^{4′}), (I^{5′}), (I⁶′), (I^{7a}′) or (I^{7b}′)or (I^{8′}), as described below.

In a particular embodiment the following compounds are excluded:

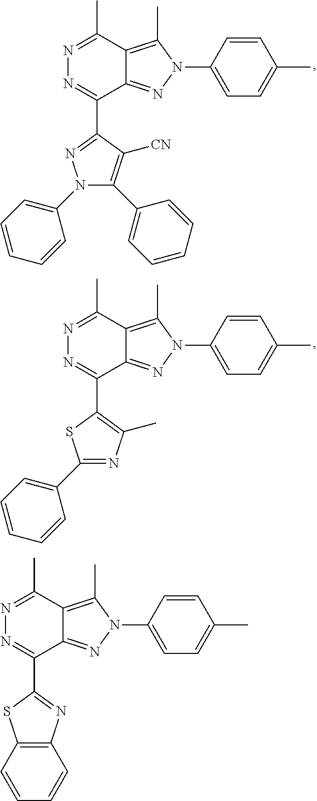

In a further embodiment the compound according to the invention of general Formula (I) is a compound of general Formula (I′)

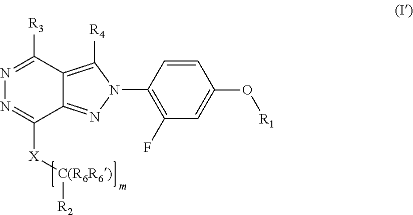

In a further embodiment the compound according to the invention of general Formula (I) or (In) is a compound of general Formula (I′), (I²′), (I³′), (I^{4′}), (I^{5′}), (I⁶′), (I^{7a}′) or (I^{7b}′) or (I^{8′}),

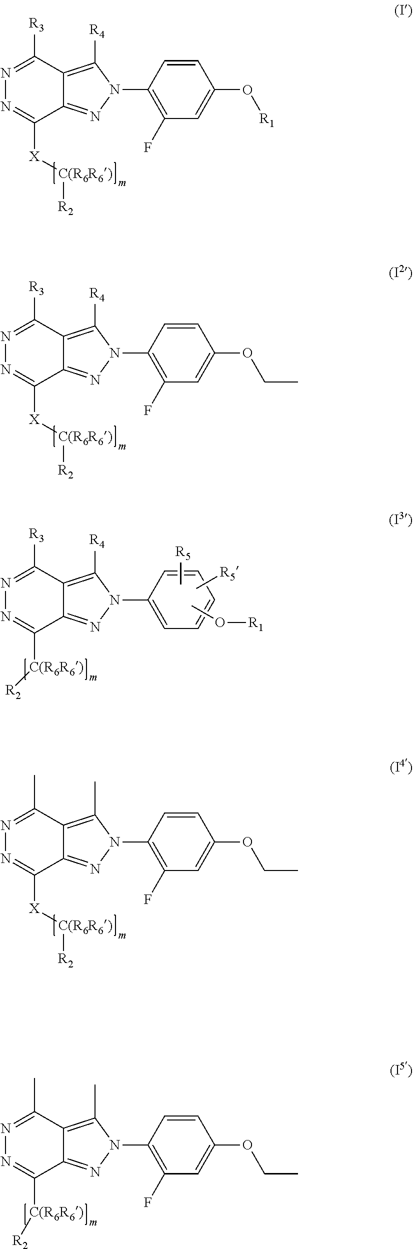

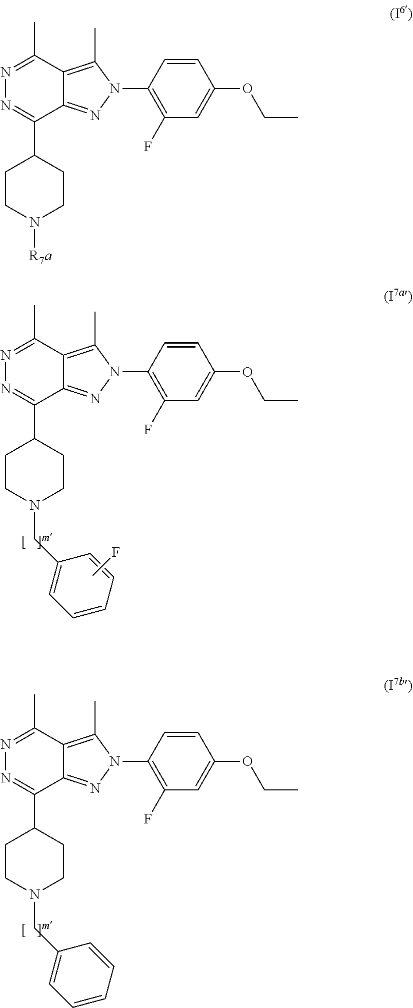

wherein R₁, R₂, R₃, R₄, R₅, R_{5′}, R₆, R_{6′}, R₇ₐ, X and m, m′ are as defined below in the detailed description,

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I′)

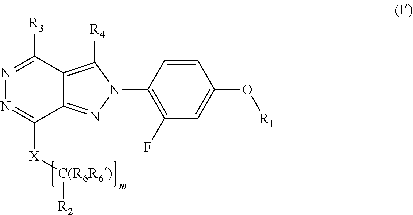

wherein

m is 0, 1, 2, 3, 4 or 5;

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl;
Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;

R_{x′}, is selected from hydrogen, substituted or unsubstituted alkylaryl, substituted or unsubstituted alkylcycloalkyl and substituted or unsubstituted alkylheterocyclyl;

wherein R₈ is selected from hydrogen, substituted or unsubstituted C₁₋₆ alkyl, substituted or unsubstituted C₂₋₆ alkenyl and substituted or unsubstituted C₂₋₆ alkynyl;

R₁ is selected from substituted or unsubstituted C₁₋₆ alkyl, substituted or unsubstituted C₂₋₆ alkenyl and substituted or unsubstituted C₂₋₆ alkynyl;

R₂ is selected from —NR₇R_{7′″}, —CN and substituted or unsubstituted N-containing-heterocyclyl;

R₃ is selected from hydrogen, substituted or unsubstituted C₁₋₆ alkyl, substituted or unsubstituted C₂₋₆ alkenyl and substituted or unsubstituted C₂₋₆ alkynyl;

R₄ is selected from hydrogen, substituted or unsubstituted C₁₋₆ alkyl, substituted or unsubstituted C₂₋₆ alkenyl and substituted or unsubstituted C₂₋₆ alkynyl;

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I²′)

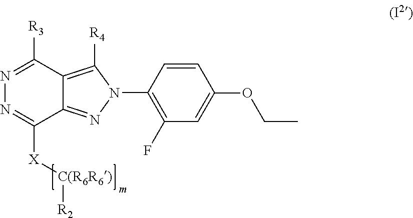

wherein

m is 0, 1, 2, 3, 4 or 5;

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl;
Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;
R_{x′}, is selected from hydrogen, substituted or unsubstituted alkylaryl, substituted or unsubstituted alkylcycloalkyl and substituted or unsubstituted alkylheterocyclyl;
wherein R₈ is selected from hydrogen, substituted or unsubstituted C₁₋₆ alkyl, substituted or unsubstituted C₂₋₆ alkenyl and substituted or unsubstituted C₂₋₆ alkynyl;

R₂ is selected from —NR₇R_{7′″}, —CN and substituted or unsubstituted N-containing-heterocyclyl;

R₃ is selected from hydrogen, substituted or unsubstituted C₁₋₆ alkyl, substituted or unsubstituted C₂₋₆ alkenyl and substituted or unsubstituted C₂₋₆ alkynyl;

R₄ is selected from hydrogen, substituted or unsubstituted C₁₋₆ alkyl, substituted or unsubstituted C₂₋₆ alkenyl and substituted or unsubstituted C₂₋₆ alkynyl;

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I³′)

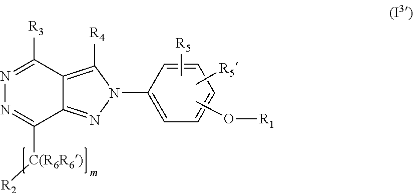

wherein

m is 0, 1, 2, 3, 4 or 5;

R₁ is selected from substituted or unsubstituted C₁₋₆ alkyl, substituted or unsubstituted C₂₋₆ alkenyl and substituted or unsubstituted C₂₋₆ alkynyl;

R₂ is selected from —NR₇R_{7′″}, —CN and substituted or unsubstituted N-containing-heterocyclyl;

R₃ is selected from hydrogen, substituted or unsubstituted C₁₋₆ alkyl, substituted or unsubstituted C₂₋₆ alkenyl and substituted or unsubstituted C₂₋₆ alkynyl;

R₄ is selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9″}, —R₉ , —S(O)R₉, S(O)₂R₉, —CN, haloalkyl, haloalkoxy, —C(O)OR₉, —C(O)NR₉R_{9′}, —NR₉S(O)₂NR_{9′}R_{9″} and C(CH₃)₂OR₉;
wherein R₉, R_{9′} and R_{9″} are independently selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I⁴′)

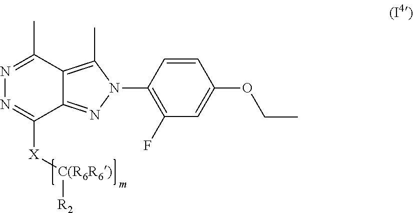

wherein m is 0, 1, 2, 3, 4 or 5;

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl;
Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;
R_{x′}, is selected from hydrogen, substituted or unsubstituted alkylaryl, substituted or unsubstituted alkylcycloalkyl and substituted or unsubstituted alkylheterocyclyl;
wherein R₈ is selected from hydrogen, substituted or unsubstituted C₁₋₆ alkyl, substituted or unsubstituted C₂₋₆ alkenyl and substituted or unsubstituted C₂₋₆ alkynyl;

R₂ is selected from —NR₇R_{7′″}, —CN and substituted or unsubstituted N-containing-heterocyclyl;

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I⁵′)

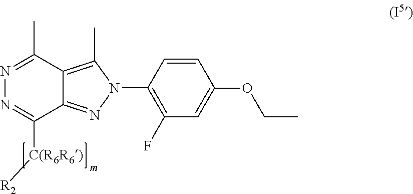

wherein

m is 0, 1, 2, 3, 4 or 5;

R₂ is selected from —NR₇R_{7′″}, —CN and substituted or unsubstituted N-containing-heterocyclyl;

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I⁶′)

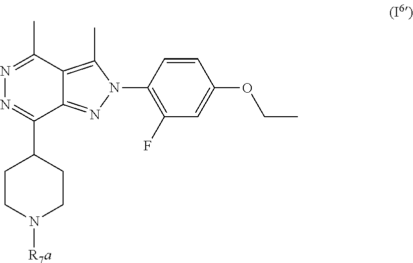

wherein R₇ₐ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I^{7a}′)

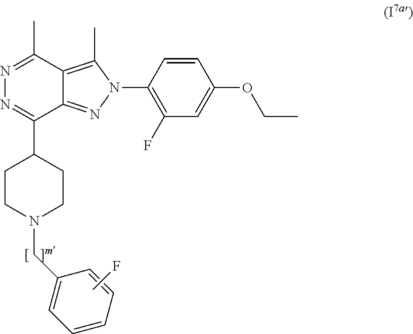

wherein

m′ is 1 or 2

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound of general Formula (I^{7b}′)

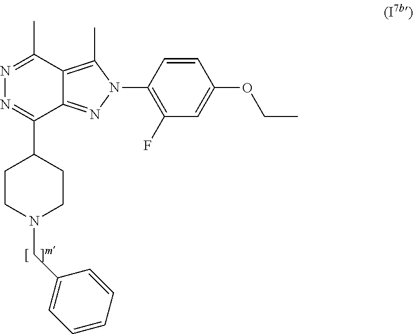

wherein

m′ is 1 or 2

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

For clarity purposes, reference is also made to the following statements below in the definitions of substitutions on alkyl etc. or aryl etc. that “wherein when different radicals R₁ to R_{14′′″} and Rₓ, R_{x′}, are present simultaneously in Formula I they may be identical or different”. This statement is reflected in the below general Formula (I^{8′}) being derived from and falling into general Formula (I) as well as Formula (I).

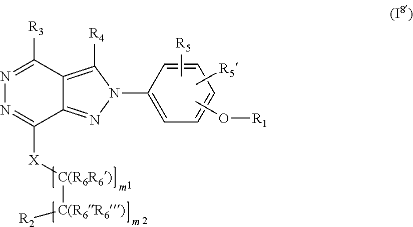

wherein R₁, R₂, R₃, R₄, R₅, R_{5′}, R₆, R_{6′} and X are as defined in the description. In addition, m¹ (being 0 or 1), m² (being 0, 1, 2, 3 or 4), R_{6″} and R_{6′″} are added. As said above, this statement is thus reflected in that R_{6″} and R_{6′″} are or could be different from R₆ and R_{6′} or not and—accordingly—m² being 0, 1, 2, 3 or 4 is naturally resulting from m (in general Formula (In), (I) or (I′) being 0, 1, 2, 3, 4 or 5).

The same would be applicable mutatis mutandis for general Markush Formulas like general Formula (I) as well as the other general Markush Formulas (I^{′}) to (I^{7′}), (I^{7a′}), (I^{7b′}) and (In) above as well as to all the intermediates of synthesis.

For clarity purposes, all groups and definitions described in the present description and referring to compounds of general Markush Formula (I), also apply to compounds of general Markush Formulae (In), (I′), (I²′), (I³′), (I^{4′}), (I_{5′}), (I⁶′), (I^{7a}′) and (I^{7b}′) and (I^{8′}), (where applicable), as well as to all the intermediates of synthesis, since compounds of general Markush Formulae (I′), (I²′), (I³′), (I^{4′}), (I^{5′}), (I⁶′), (I^{7a}′) and (I^{7b}′) and (I^{8′}), are included within the scope of the larger definition of general Markush Formula (I).

For clarity purposes, the general Markush Formula (I)

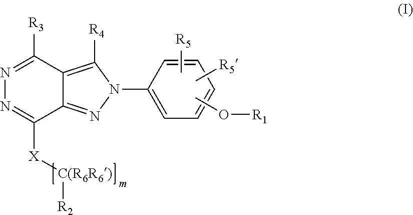

is equivalent to

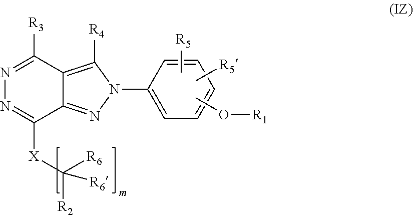

wherein only —C(R₆R_{6′})— is included into the brackets, and m means the number of times that —C(R₆R_{6′})— is repeated. The same would apply, when applicable, to general Markush Formulae (In), (I′), (I²′), (I³′), (I^{4′}), (I^{5′}), (I⁶′), (I^{7a}′), (I^{7b}′) and (I^{8′}) and also to all the intermediates of synthesis.

In addition, and for clarity purposes, it should further be understood that naturally if m is 0, R₂ and/or X are still present, when applicable, in general Markush Formulae (I), (In), (I′), (I²′), (I³′), (I^{4′}), (I^{5′}), (I⁶′), (I^{7a}′), (I^{7b}′) and (I^{8′}) and to all the intermediates of synthesis.

For clarity purposes, a compound of Formula (Ia), is a compound of Formula (I)

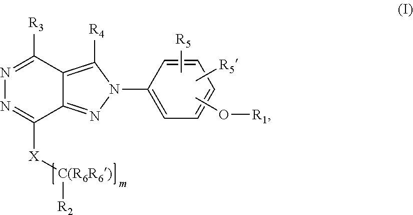

wherein X is —HC═CH—,

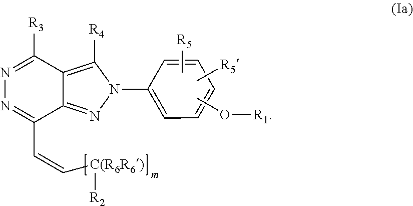

For clarity purposes, a compound of Formula (Ib), is a compound of Formula (I)

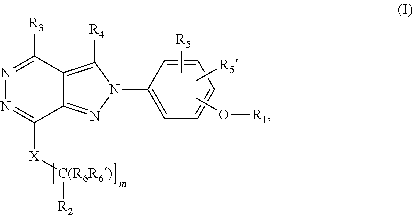

wherein X is —CH₂CH₂—,

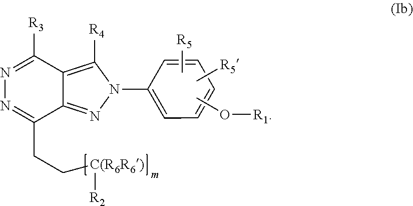

In the context of this invention, alkyl is understood as meaning saturated, linear or branched hydrocarbons, which may be unsubstituted or substituted once or several times. It encompasses e.g. —CH₃ and —CH₂—CH₃. In these radicals, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4-, or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₈-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C10-alkyl and C₁₋₁₈-alkyl represents C1-, C2-, C3-, C4-, C5-, C6-, C7-, C8-, C9-, C10-, C11-, C12-, C13-, C14-, C15-, C16-, C17- or C18-alkyl. The alkyl radicals are preferably methyl, ethyl, propyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, if substituted also CHF₂, CF₃ or CH₂OH etc. Preferably alkyl is understood in the context of this invention as C₁₋₈alkyl like methyl, ethyl, propyl, butyl, pentyl, hexyl, heptyl, or octyl; preferably is C₁₋₆alkyl like methyl, ethyl, propyl, butyl, pentyl, or hexyl; more preferably is C₁₋₄alkyl like methyl, ethyl, propyl or butyl.

Alkenyl is understood as meaning unsaturated, linear or branched hydrocarbons, which may be unsubstituted or substituted once or several times. It encompasses groups like e.g. —CH═CH—CH₃. The alkenyl radicals are preferably vinyl (ethenyl), allyl (2-propenyl). Preferably in the context of this invention alkenyl is C₂₋₁₀-alkenyl or C₂₋₈-alkenyl like ethylene, propylene, butylene, pentylene, hexylene, heptylene or octylene; or is C₂₋₆-alkenyl like ethylene, propylene, butylene, pentylene, or hexylene; or is C₂₋₄-alkenyl, like ethylene, propylene, or butylenes.

Alkynyl is understood as meaning unsaturated, linear or branched hydrocarbons, which may be unsubstituted or substituted once or several times. It encompasses groups like e.g. —C═C—CH₃ (1-propinyl). Preferably alkynyl in the context of this invention is C₂₋₁₀-alkynyl or C₂₋₈-alkynyl like ethyne, propyne, butyene, pentyne, hexyne, heptyne, or octyne; or is C₂₋₆-alkynyl like ethyne, propyne, butyene, pentyne, or hexyne; or is C₂₋₄-alkynyl like ethyne, propyne, butyene, pentyne, or hexyne.

In connection with alkyl (also in alkylaryl, alkylheterocyclyl or alkylcycloalkyl), alkenyl, alkynyl and O-alkyl —unless defined otherwise—the term substituted in the context of this invention is understood as meaning replacement of at least one hydrogen radical on a carbon atom by halogen (F, Cl, Br, I), —NRₖR_{k′″}, —SRₖ, —S(O)Rₖ, —S(O)₂Rₖ, —ORₖ, —C(O)Rₖ, —C(O)ORₖ, —CN, —C(O)NRₖR_{k′}, haloalkyl, haloalkoxy , being Rₖ represented by R₁₁ or R₁₂ or R₁₃, (being R_{k′} represented by R_{11′}, or R_{12′} or R_{13′}; being R_{k″} represented by R_{11″} or R_{12″} or R_{13″}; being R_{k′″} represented by R_{11′″} or R_{12′″} or R_{13′″}), wherein R₁ to R_{14′″} and Rₓ and R_{x′}, are as defined in the description, and wherein when different radicals R₁ to R_{14′″} and Rₓ and R_{x′}, are present simultaneously in Formula I they may be identical or different.

Most preferably in connection with alkyl (also in alkylaryl, alkylheterocyclyl or alkylcycloalkyl), alkenyl, alkynyl or O-alkyl, substituted is understood in the context of this invention that any alkyl (also in alkylaryl, alkylheterocyclyl or alkylcycloalkyl), alkenyl, alkynyl or O-alkyl which is substituted is substituted with one or more of halogen (F, Cl, Br, I), —ORₖ, —C(O)Rₖ, —CN, —SRₖ, —S(O)Rₖ, and —S(O)₂Rₖ, haloalkyl, haloalkoxy being Rₖ represented by R₁₁ or R₁₂ or R₁₃, (being R_{k′} represented by R_{11′} or R_{12′} or R_{13′}; being R_{k″} represented by R_{11″} or R_{12″} or R_{13″}; being R_{k′″} represented by R_{11′″} or R_{12′″} or R_{13′″} , wherein R₁ to R_{14′″} and Rₓ and R_{x′}, are as defined in the description, and wherein when different radicals R₁ to R_{14′″} and Rₓ and R_{x′} are present simultaneously in Formula I they may be identical or different.

More than one replacement on the same molecule and also on the same carbon atom is possible with the same or different substituents. This includes for example 3 hydrogens being replaced on the same C atom, as in the case of CF₃, or at different places of the same molecule, as in the case of e.g. —CH(OH)—CH═CH—CHCl₂.

In the context of this invention haloalkyl is understood as meaning an alkyl being substituted once or several times by a halogen (selected from F, Cl, Br, I). It encompasses e.g. —CH₂Cl, —CH₂F, —CHCl₂, —CHF₂, —CCl₃, —CF₃ and —CH₂—CHCl₂. Preferably haloalkyl is understood in the context of this invention as halogen-substituted C₁₋₄-alkyl representing halogen substituted C1-, C2-, C3- or C4-alkyl. The halogen-substituted alkyl radicals are thus preferably methyl, ethyl, propyl, and butyl. Preferred examples include —CH₂Cl, —CH₂F, —CHCl₂, —CHF₂, and —CF₃.

In the context of this invention haloalkoxy is understood as meaning an —O-alkyl being substituted once or several times by a halogen (selected from F, Cl, Br, I). It encompasses e.g. —OCH₂C, —OCH₂F, —OCHCl₂, —OCHF₂, —OCCl₃, —OCF₃ and —OCH₂—CHCl₂. Preferably haloalkyl is understood in the context of this invention as halogen-substituted —OC₁₋₄-alkyl representing halogen substituted C1-, C2-, C3- or C4-alkoxy. The halogen-substituted alkyl radicals are thus preferably O-methyl, O-ethyl, O-propyl, and O-butyl. Preferred examples include —OCH₂Cl, —OCH₂F, —OCHCl₂, —OCHF₂, and —OCF₃.

In the context of this invention cycloalkyl is understood as meaning saturated and unsaturated (but not aromatic) cyclic hydrocarbons (without a heteroatom in the ring), which can be unsubstituted or once or several times substituted. Furthermore, C₃₋₄-cycloalkyl represents C3- or C4-cycloalkyl, C₃₋₅-cycloalkyl represents C3-, C4- or C5-cycloalkyl, C₃₋₆-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C₄₋₆-cycloalkyl represents C4-, C5- or C6-cycloalkyl, C₄₋₇-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. Examples are cyclopropyl, 2-methylcyclopropyl, cyclopropylmethyl, cyclobutyl, cyclopentyl, cyclopentylmethyl, cyclohexyl, cycloheptyl, cyclooctyl, and also adamantly. Preferably in the context of this invention cycloalkyl is C₃₋₈cycloalkyl like cyclopropyl, cyclobutyl, cyclopentyl, cyclohexyl, cycloheptyl, or cyclooctyl; or is C₃₋₇cycloalkyl like cyclopropyl, cyclobutyl, cyclopentyl, cyclohexyl, or cycloheptyl; or is C₃₋₆cycloalkyl like cyclopropyl, cyclobutyl, cyclopentyl or cyclohexyl, especially cyclopentyl or cyclohexyl.

Aryl is understood as meaning 5 to 18 membered mono or polycyclic ring systems with at least one aromatic ring but without heteroatoms even in only one of the rings. Examples are phenyl, naphthyl, fluoranthenyl, fluorenyl, tetralinyl or indanyl, 9H-fluorenyl or anthracenyl radicals, which can be unsubstituted or once or several times substituted. Most preferably aryl is understood in the context of this invention as phenyl, naphthyl or anthracenyl, preferably is phenyl.

A heterocyclyl radical or group (also called heterocyclyl hereinafter) is understood as meaning 5 to 18 membered mono or polycyclic heterocyclic ring systems, with at least one saturated or unsaturated ring which contains one or more heteroatoms selected from the group consisting of nitrogen, oxygen and/or sulfur in the ring. A heterocyclic group can also be substituted once or several times.

Examples include non-aromatic heterocyclyls such as tetrahydropyran, oxazepane, morpholine, piperidine, pyrrolidine as well as heteroaryls such as furan, benzofuran, thiophene, benzothiophene, pyrrole, pyridine, pyrimidine, pyrazine, quinoline, isoquinoline, phthalazine, thiazole, benzothiazole, indole, benzotriazole, carbazole and quinazoline.

Subgroups inside the heterocyclyls as understood herein include heteroaryls and non-aromatic heterocyclyls.
the heteroaryl (being equivalent to heteroaromatic radicals or aromatic heterocyclyls) is an aromatic 5 to 18 membered mono or polycyclic heterocyclic ring system of one or more rings of which at least one aromatic ring contains one or more heteroatoms selected from the group consisting of nitrogen, oxygen and/or sulfur in the ring; preferably is an 5 to 18 membered mono or polyclic aromatic heterocyclic ring system of one or two rings of which at least one aromatic ring contains one or more heteroatoms selected from the group consisting of nitrogen, oxygen and/or sulfur in the ring, more preferably is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, imidazole, pyrazole, oxazole, thiophene and benzimidazole;
the non-aromatic heterocyclyl is a 5 to 18 membered mono or polycyclic heterocyclic ring system of one or more rings of which at least one ring —with this (or these) ring(s) then not being aromatic—contains one or more heteroatoms selected from the group consisting of nitrogen, oxygen and/or sulfur in the ring; preferably is a 5 to 18 membered mono or polycyclic heterocyclic ring system of one or two rings of which one or both rings—with this one or two rings then not being aromatic—contain/s one or more heteroatoms selected from the group consisting of nitrogen, oxygen and/or sulfur in the ring, more preferably is selected from oxazepam, pyrrolidine, piperidine, piperazine, tetrahydropyran, morpholine, indoline, oxopyrrolidine, benzodioxane, especially is benzodioxane, morpholine, tetrahydropyran, piperidine, oxopyrrolidine and pyrrolidine.

Preferably in the context of this invention heterocyclyl is defined as a 5 to 18 membered mono or polycyclic heterocyclic ring system of one or more saturated or unsaturated rings of which at least one ring contains one or more heteroatoms selected from the group consisting of nitrogen, oxygen and/or sulfur in the ring. Preferably it is a 5 to 18 membered mono or polycyclic heterocyclic ring system of one or two saturated or unsaturated rings of which at least one ring contains one or more heteroatoms selected from the group consisting of nitrogen, oxygen and/or sulfur in the ring.

Preferred examples of heterocyclyls include oxazepan, pyrrolidine, imidazole, oxadiazole, tetrazole, pyridine, pyrimidine, piperidine, piperazine, benzofuran, benzimidazole, indazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, tetrahydroisoquinoline, phthalazine, benzo-1,2,5-thiadiazole, indole, benzotriazole, benzoxazole oxopyrrolidine, pyrimidine, benzodioxolane, benzodioxane, carbazole and quinazoline, especially is pyridine, pyrazine, indazole, benzodioxane, thiazole, benzothiazole, morpholine, tetrahydropyran, pyrazole, imidazole, piperidine, thiophene, indole, benzimidazole, pyrrolo[2,3b]pyridine, benzoxazole, oxopyrrolidine, pyrimidine, oxazepane and pyrrolidine.

In the context of this invention oxopyrrolidine is understood as meaning pyrrolidin-2-one.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

In the context of this invention alkylaryl is understood as meaning an aryl group (see above) being connected to another atom through a C₁₋₆-alkyl (see above) which may be branched or linear and is unsubstituted or substituted once or several times. Preferably alkylaryl is understood as meaning an aryl group (see above) being connected to another atom through 1 to 4 (—CH₂—) groups. Most preferably alkylaryl is benzyl (i.e. —CH₂-phenyl).

In the context of this invention alkylheterocyclyl is understood as meaning an heterocyclyl group being connected to another atom through a C₁₋₆-alkyl (see above) which may be branched or linear and is unsubstituted or substituted once or several times. Preferably alkylheterocyclyl is understood as meaning an heterocyclyl group (see above) being connected to another atom through 1 to 4 (—CH₂—) groups. Most preferably alkylheterocyclyl is —CH₂-pyridine.

In the context of this invention alkylcycloalkyl is understood as meaning an cycloalkyl group being connected to another atom through a C₁₋₆-alkyl (see above) which may be branched or linear and is unsubstituted or substituted once or several times. Preferably alkylcycloalkyl is understood as meaning an cycloalkyl group (see above) being connected to another atom through 1 to 4 (—CH₂—) groups. Most preferably alkylcycloalkyl is —CH₂-cyclopropyl.

Preferably, the aryl is a monocyclic aryl. More preferably the aryl is a 5, 6 or 7 membered monocyclic aryl. Even more preferably the aryl is a 5 or 6 membered monocyclic aryl.

Preferably, the heteroaryl is a monocyclic heteroaryl. More preferably the heteroaryl is a 5, 6 or 7 membered monocyclic heteroaryl. Even more preferably the heteroaryl is a 5 or 6 membered monocyclic heteroaryl.

Preferably, the non-aromatic heterocyclyl is a monocyclic non-aromatic heterocyclyl. More preferably the non-aromatic heterocyclyl is a 4, 5, 6 or 7 membered monocyclic non-aromatic heterocyclyl. Even more preferably the non-aromatic heterocyclyl is a 5 or 6 membered monocyclic non-aromatic heterocyclyl.

Preferably, the cycloalkyl is a monocyclic cycloalkyl. More preferably the cycloalkyl is a 3, 4, 5, 6, 7 or 8 membered monocyclic cycloalkyl. Even more preferably the cycloalkyl is a 3, 4, 5 or 6 membered monocyclic cycloalkyl.

In connection with aryl (including alkyl-aryl), cycloalkyl (including alkyl-cycloalkyl), or heterocyclyl (including alkyl-heterocyclyl), substituted is understood—unless defined otherwise—as meaning substitution of the ring-system of the aryl or alkyl-aryl, cycloalkyl or alkyl-cycloalkyl; heterocyclyl or alkyl-heterocyclyl with one or more of halogen (F, Cl, Br, I), —Rₖ , —ORₖ, —CN, —NO₂, —NRₖR_{k′″}, —C(O)ORₖ, NRₖC(O)R_{k′}, —C(O)NRₖR_{k′}, —NRₖS(O)₂R_{k′}, ═O, —OCH₂CH₂OH, —NRₖC(O)NR_{k′}R_{k″}, —S(O)₂NRₖR_{k″}, —NRₖS(O)₂NR_{k′}R_{k″}, haloalkyl, haloalkoxy, —SRₖ, —S(O)Rₖ, —S(O)₂Rₖ or C(CH₃)ORₖ; NRₖR_{k′″}, substituted or unsubstituted alkylcycloalkyl, substituted or unsubstituted alkylaryl, substituted or unsubstituted alkylheterocyclyl, with Rₖ R_{k′}, R_{k″}, R_{k′″} and R_{k′′″} independently being either H or a saturated or unsaturated, linear or branched, substituted or unsubstituted C₁₋₆-alkyl; a saturated or unsaturated, linear or branched, substituted or unsubstituted C₁₋₆-alkyl; a saturated or unsaturated, linear or branched, substituted or unsubstituted —O—C₁₋₆-alkyl (alkoxy); a saturated or unsaturated, linear or branched, substituted or unsubstituted —S—C₁₋₆-alkyl; a saturated or unsaturated, linear or branched, substituted or unsubstituted —C(O)—C_{1-6—}alkyl-group; a saturated or unsaturated, linear or branched, substituted or unsubstituted —C(O)—O—C₁₋₆-alkyl-group; a substituted or unsubstituted aryl or alkyl-aryl; a substituted or unsubstituted cycloalkyl or alkyl-cycloalkyl; a substituted or unsubstituted heterocyclyl or alkyl-heterocyclyl, being Rₖ one of R₁₂ or R₁₄ or R₇ₐ, (being R_{k′} one of R_{12′} or R_{14′}; being R_{k″} one of R_{12″} or R_{14″}; being R_{k′″} one of R_{12′″} or R_{14′″}, wherein R₁ to R_{14′″} and Rₓ and R_{x′}, are as defined in the description, and wherein when different radicals R₁ to R_{14′″} and Rₓ and R_{x′} are present simultaneously in Formula I they may be identical or different.

Most preferably in connection with aryl (including alkyl-aryl), cycloalkyl (including alkyl-cycloalkyl), or heterocyclyl (including alkyl-heterocyclyl), substituted is understood in the context of this invention that any aryl, cycloalkyl and heterocyclyl which is substituted is substituted (also in an alyklaryl, alkylcycloalkyl or alkylheterocyclyl) with one or more of halogen (F, Cl, Br, I), —Rₖ ,—ORₖ, —CN , —NO₂ , —NRₖR_{k′″}, NRₖC(O)R_{k′}, —NRₖS(O)₂R_{k′}, —S(O)₂NRₖR_{k′}, —NRₖC(O)NR_{k′}R_{k″}, haloalkyl, haloalkoxy, —SRₖ , —S(O)Rₖ or S(O)₂Rₖ; being Rₖ one of R₁₂ or R₁₄ or R₇ₐ, (being R_{k′} one of R_{12′} or R_{14′}; being R_{k″} one of R_{12″} or R_{14″}; being R_{k″} one of R_{12′″} or R_{14′″}, wherein R₁ to R_{14′″} and Rₓ and R_{x′} are as defined in the description, and wherein when different radicals R₁ to R_{14′″} and R_{x′} and R_{x′}, are present simultaneously in Formula I they may be identical or different.

In connection with cycloalkyl (including alkyl-cycloalkyl), or heterocycly (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl with [symbol] (leading to a spiro structure) or with ═O.

A ring system is a system consisting of at least one ring of connected atoms but including also systems in which two or more rings of connected atoms are joined with “joined” meaning that the respective rings are sharing one (like a spiro structure), two or more atoms being a member or members of both joined rings.

The term “leaving group” means a molecular fragment that departs with a pair of electrons in heterolytic bond cleavage. Leaving groups can be anions or neutral molecules. Common anionic leaving groups are halides such as Cl—, Br—, and I—, and sulfonate esters, such as tosylate (TsO—) or mesylate.

The term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes via ionic interactions.

The term “physiologically acceptable salt” means in the context of this invention any salt that is physiologically tolerated (most of the time meaning not being toxic—especially not caused by the counter-ion) if used appropriately for a treatment especially if used on or applied to humans and/or mammals.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH₄, but in particular (mono)- or (di)sodium, (mono)- or (di)potassium, magnesium or calcium salts.

Physiologically acceptable salts can also be formed with anions or acids and in the context of this invention is understood as meaning salts of at least one of the compounds used according to the invention as the cation with at least one anion which are physiologically tolerated—especially if used on humans and/or mammals. By this is understood in particular, in the context of this invention, the salt formed with a physiologically tolerated acid, that is to say salts of the particular active compound with inorganic or organic acids which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The compounds of the invention may be present in crystalline form or in the form of free compounds like a free base or acid.

Any compound that is a solvate of a compound according to the invention like a compound according to general formula I defined above is understood to be also covered by the scope of the invention. Methods of solvation are generally known within the art. Suitable solvates are pharmaceutically acceptable solvates. The term “solvate” according to this invention is to be understood as meaning any form of the active compound according to the invention in which this compound has attached to it via non-covalent binding another molecule (most likely a polar solvent). Especially preferred examples include hydrates and alcoholates, like methanolates or ethanolates.

Any compound that is a prodrug of a compound according to the invention like a compound according to general formula I defined above is understood to be also covered by the scope of the invention. The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the present compounds: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a N-oxide of a compound according to the invention like a compound according to general formula I defined above is understood to be also covered by the scope of the invention.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by ¹³C— or ¹⁴C-enriched carbon or of a nitrogen by ¹⁵N-enriched nitrogen are within the scope of this invention. This would especially also apply to the provisos described above so that any mentioning of hydrogen or any “H” in a formula would also cover deuterium or tritium.

The compounds of formula (I) as well as their salts or solvates of the compounds are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

In a further aspect, the present invention is directed to compounds of general Formula (In):

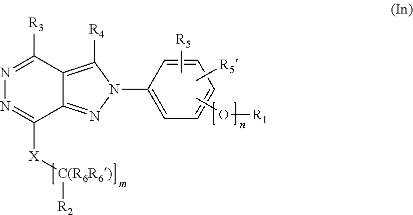

wherein

m is 0, 1, 2, 3, 4 or 5;

n is 0 or 1;

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl; wherein
Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;
R_{x′} is selected from hydrogen, substituted or unsubstituted alkylaryl, substituted or unsubstituted alkylcycloalkyl and substituted or unsubstituted alkylheterocyclyl;
wherein R₈ is selected from hydrogen, substituted or unsubstituted C₁₋₆ alkyl, substituted or unsubstituted C₂₋₆ alkenyl and substituted or unsubstituted C₂₋₆ alkynyl;

R₁ is selected from substituted or unsubstituted C₁₋₆ alkyl, substituted or unsubstituted C₂₋₆ alkenyl and substituted or unsubstituted C₂₋₆ alkynyl;
wherein the alkyl, alkenyl or alkynyl in R₁, if substituted, is substituted with one or more substituent/s selected from —OR₁₁, —C(O)R₁₁, halogen, —CN, haloalkyl, haloalkoxy and —NR₁₁R_{11′″};
wherein R₁₁ is selected from hydrogen, unsubstituted C₁₋₆ alkyl, unsubstituted C₂₋₆ alkenyl and unsubstituted C₂₋₆ alkynyl;
and R_{11′″} is selected from hydrogen, unsubstituted C₁₋₆ alkyl, unsubstituted C₂₋₆ alkenyl, unsubstituted C₂₋₆ alkynyl and -Boc;

R₂ is selected from —NR₇R_{7′″}, —CN and substituted or unsubstituted N-containing-heterocyclyl;
wherein said N-containing-heterocyclyl in R₂, if substituted, is mono-substituted with R₇ₐ;
wherein R₇ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc;
and wherein R_{7′″}, is selected from hydrogen, substituted or unsubstituted C₁₋₆ alkyl, substituted or unsubstituted C₂₋₆ alkenyl and substituted or unsubstituted C₂₋₆ alkynyl;
wherein R₇ₐ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl and -Boc;
wherein, the alkyl, alkenyl or alkynyl, also in alkylaryl, alkyheterocyclyl or alkycycloalkyl, in R₇ₐ, if substituted, is substituted with one or more substituent/s selected from —OR₁₂, halogen, —CN, haloalkyl, haloalkoxy and —NR₁₂R_{12′″};

and wherein, the aryl, heterocyclyl or cycloalkyl, also in alkylaryl, alkyheterocyclyl or alkycycloalkyl, in R₇ₐ, if substituted, is substituted with one or more substituent/s selected from halogen, —R₁₂, —OR₁₂, —NO₂, —NR₁₂R_{12′″}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂ , —S(O)R₁₂, S(O)₂R₁₂, —CN, haloalkyl, haloalkoxy, —C(O)OR₁₂, —C(O)NR₁₂R_{12′}, —NR₁₂S(O)₂NR_{12′}, R_{12″}, unsubstituted alkylcycloalkyl, unsubstituted alkylaryl, and unsubstituted alkylheterocyclyl;
wherein R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;
and wherein R_{12′″} is selected from hydrogen, unsubstituted C₁₋₆ alkyl, unsubstituted C₂₋₆ alkenyl, unsubstituted C₂₋₆ alkynyl and -Boc;

R₃ is selected from hydrogen, substituted or unsubstituted C₁₋₆ alkyl, substituted or unsubstituted C₂₋₆ alkenyl and substituted or unsubstituted C₂₋₆ alkynyl;

R₄ is selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9′}, —SR₉ , —S(O)R₉, S(O)₂R₉, —CN, haloalkyl, haloalkoxy, —C(O)OR₉, —C(O)NR₉R_{9′}, —NR₉S(O)₂NR_{9′}R_{9″} and C(CH₃)₂—OR₉;
wherein R₉, R_{9′} and R_{9″} are independently selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl;

wherein, the alkyl, alkenyl or alkynyl, also in alkylaryl, alkyheterocyclyl or alkycycloalkyl, other than those defined in R₁, R₂ or R₇ₐ, if substituted, is substituted with one or more substituent/s selected from —OR₁₃, halogen, —CN, haloalkyl, haloalkoxy and —NR₁₃R_{13′″};
wherein R₁₃ is selected from hydrogen, unsubstituted C₁₋₆ alkyl, unsubstituted C₂₋₆ alkenyl, and unsubstituted C₂₋₆ alkynyl;
and wherein R_{13′″} is selected from hydrogen, unsubstituted C₁₋₈ alkyl, unsubstituted C₂₋₈ alkenyl, unsubstituted C₂₋₈ alkynyl and -Boc;

and wherein, the aryl, heterocyclyl or cycloalkyl, also in alkylaryl, alkyheterocyclyl or alkycycloalkyl, other than those defined in R₁, R₂ or R₇ₐ, if substituted, is substituted with one or more substituent/s selected from halogen, —R₁₄, —OR₁₄, —NO₂, —NR₁₄R_{14′″}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄ , —S(O)R₁₄, S(O)₂R₁₄, —CN, haloalkyl, haloalkoxy, —C(O)OR₁₄, —C(O)NR₁₄R_{14′}, —NR₁₄S(O)₂NR_{14′}R_{14″} and C(CH₃)₂OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;
and wherein R_{14′″} is selected from hydrogen, unsubstituted C₁₋₆ alkyl, unsubstituted C₂₋₆ alkenyl, unsubstituted C₂₋₆ alkynyl and -Boc;

with the proviso that point of attachment of the —X—[CR₆R_{6′}]ₘ—R₂ moiety to the pyrazolopyridazine structure is not represented by a nitrogen atom;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof;

In a further aspect, the present invention is directed to compounds of general Formula (In):

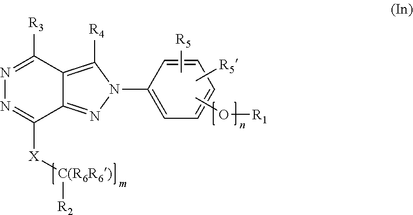

wherein

m is 0, 1, 2, 3, 4 or 5;

n is 0 or 1;

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl; wherein
Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;
Rₓ is selected from hydrogen, substituted or unsubstituted alkylaryl, substituted or unsubstituted alkylcycloalkyl and substituted or unsubstituted alkylheterocyclyl;
wherein R₈ is selected from hydrogen, substituted or unsubstituted C₁₋₆ alkyl, substituted or unsubstituted C₂₋₆ alkenyl and substituted or unsubstituted C₂₋₆alkynyl;

R₁ is selected from substituted or unsubstituted C₁₋₆ alkyl, substituted or unsubstituted C₂₋₆ alkenyl and substituted or unsubstituted C₂₋₆ alkynyl;
wherein the alkyl, alkenyl or alkynyl in R₁, if substituted, is substituted with one or more substituent/s selected from —OR₁₁, —C(O)R₁₁, halogen, —CN, haloalkyl, haloalkoxy and —NR₁₁R_{11′″};
wherein R₁₁ is selected from hydrogen, unsubstituted C₁₋₆ alkyl, unsubstituted C₂₋₆ alkenyl and unsubstituted C₂₋₆ alkynyl;
and R_{11′″} is selected from hydrogen, unsubstituted C₁₋₆ alkyl, unsubstituted C₂₋₆ alkenyl, unsubstituted C₂₋₆ alkynyl and -Boc;

R₂ is selected from —NR₇R_{7′″}, —CN and substituted or unsubstituted N-containing-heterocyclyl;
wherein said N-containing-heterocyclyl in R₂, if substituted, is mono-substituted with R₇ₐ;
wherein R₇ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc;
and wherein R_{7′″} is selected from hydrogen, substituted or unsubstituted C₁₋₆ alkyl, substituted or unsubstituted C₂₋₆ alkenyl and substituted or unsubstituted C₂₋₆ alkynyl;
wherein R₇ₐ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl and -Boc;
wherein, the alkyl, alkenyl or alkynyl, also in alkylaryl, alkyheterocyclyl or alkycycloalkyl, in R₇ₐ, if substituted, is substituted with one or more substituent/s selected from —OR₁₂, halogen, —CN, haloalkyl, haloalkoxy and —NR₁₂R_{12′″};
and wherein, the aryl, heterocyclyl or cycloalkyl, also in alkylaryl, alkyheterocyclyl or alkycycloalkyl, in R₇ₐ, if substituted, is substituted with one or more substituent/s selected from halogen, —R₁₂, —OR₁₂, —NO₂, —NR₁₂R_{12′″}, NR₁₂C(O)R_{12′}, —NR₁₂S(O)₂R_{12′}, —S(O)₂NR₁₂R_{12′}, —NR₁₂C(O)NR_{12′}R_{12′}, —SR₁₂, —S(O)R₁₂, S(O)₂R₁₂, —CN, haloalkyl, haloalkoxy, —C(O)OR₁₂, —C(O)NR₁₂R_{12′}, —NR₁₂S(O)₂NR_{12′}R_{12″}, unsubstituted alkylcycloalkyl, unsubstituted alkylaryl, and unsubstituted alkylheterocyclyl;
wherein R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;
and wherein R_{12′″} is selected from hydrogen, unsubstituted C₁₋₆ alkyl, unsubstituted C₂₋₆ alkenyl, unsubstituted C₂₋₆ alkynyl and -Boc;

R₃ is selected from hydrogen, substituted or unsubstituted C₁₋₆ alkyl, substituted or unsubstituted C₂₋₆ alkenyl and substituted or unsubstituted C₂₋₆ alkynyl;

R₄ is selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9″}, —SR₉ , —S(O)R₉, S(O)₂R₉, —CN, haloalkyl, haloalkoxy, —C(O)OR₉, —C(O)NR₉R_{9′}, —NR₉S(O)₂NR_{9′}R_{9″} and C(CH₃)₂—OR₉;
wherein R₉, R_{9′} and R_{9″} are independently selected from hydrogen, unsubstituted C₁₋₆ alkyl, unsubstituted C₂₋₆ alkenyl, and unsubstituted C₂₋₆ alkynyl;
and wherein R_{9′″} selected from hydrogen, unsubstituted C₁₋₈ alkyl, unsubstituted C₂₋₈ alkenyl, unsubstituted C₂₋₈ alkynyl and -Boc;

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl;

wherein, the alkyl, alkenyl or alkynyl, also in alkylaryl, alkyheterocyclyl or alkycycloalkyl, other than those defined in R₁, R₂ or R₇ₐ, if substituted, is substituted with one or more substituent/s selected from —OR₁₃, halogen, —CN, haloalkyl, haloalkoxy and —NR₁₃R_{13′″};
wherein R₁₃ is selected from hydrogen, unsubstituted C₁₋₆ alkyl, unsubstituted C₂₋₆ alkenyl, and unsubstituted C₂₋₆ alkynyl;
and wherein R_{13′″} is selected from hydrogen, unsubstituted C₁₋₈ alkyl, unsubstituted C₂₋₈ alkenyl, unsubstituted C₂₋₈ alkynyl and -Boc;

and wherein, the aryl, heterocyclyl or cycloalkyl, also in alkylaryl, alkyheterocyclyl or alkycycloalkyl, other than those defined in R₁, R₂ or R₇ₐ, if substituted, is substituted with one or more substituent/s selected from halogen, —R₁₄, —OR₁₄, —NO₂, —NR₁₄R_{14′″}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄ , —S(O)R₁₄, S(O)₂R₁₄, —CN, haloalkyl, haloalkoxy, —C(O)OR₁₄, —C(O)NR₁₄R—_{14′}, —NR₁₄S(O)₂NR_{14′}R_{14″} and C(CH₃)₂—OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;
and wherein R_{14′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof;

In a further embodiment the compound according to the invention has the general Formula (I)

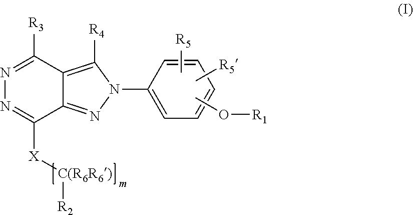

wherein

m is 0, 1, 2, 3, 4 or 5;

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl;
Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;
R_{x′}, is selected from hydrogen, substituted or unsubstituted alkylaryl, substituted or unsubstituted alkylcycloalkyl and substituted or unsubstituted alkylheterocyclyl;
wherein R₈ is selected from hydrogen, substituted or unsubstituted C₁₋₆ alkyl, substituted or unsubstituted C₂₋₆ alkenyl and substituted or unsubstituted C₂₋₆ alkynyl;

R₁ is selected from substituted or unsubstituted C₁₋₆ alkyl, substituted or unsubstituted C₂₋₆ alkenyl and substituted or unsubstituted C₂₋₆ alkynyl;
wherein the alkyl, alkenyl or alkynyl in R₁, if substituted, is substituted with one or more substituent/s selected from —OR₁₁, —C(O)R₁₁, halogen, —CN, haloalkyl, haloalkoxy and —NR₁₁R_{11′″};
wherein R₁₁ is selected from hydrogen, unsubstituted C₁₋₆ alkyl, unsubstituted C₂₋₆ alkenyl and unsubstituted C₂₋₆ alkynyl;
and R_{11′″} is selected from hydrogen, unsubstituted C₁₋₆ alkyl, unsubstituted C₂₋₆ alkenyl, unsubstituted C₂₋₆ alkynyl and -Boc;

R₂ is selected from —NR₇R_{7′″}, —CN and substituted or unsubstituted N-containing-heterocyclyl;
wherein said N-containing-heterocyclyl in R₂, if substituted, is mono-substituted with R₇ₐ;
wherein R₇ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc;
and wherein R_{7′″} is selected from hydrogen, substituted or unsubstituted C₁₋₆ alkyl, substituted or unsubstituted C₂₋₆ alkenyl and substituted or unsubstituted C₂₋₆ alkynyl;
wherein R₇ₐ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl and -Boc;
wherein, the alkyl, alkenyl or alkynyl, also in alkylaryl, alkyheterocyclyl or alkycycloalkyl, in R₇ₐ, if substituted, is substituted with one or more substituent/s selected from —OR₁₂, halogen, —CN, haloalkyl, haloalkoxy and —NR₁₂R_{12′″};
and wherein, the aryl, heterocyclyl or cycloalkyl, also in alkylaryl, alkyheterocyclyl or alkycycloalkyl, in R₇ₐ, if substituted, is substituted with one or more substituent/s selected from halogen, —R₁₂, —OR₁₂, —NO₂, —NR₁₂R_{12′″}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —NR₁₂S(O)₂NR_{12′}R_{12″}, unsubstituted alkylcycloalkyl and unsubstituted alkylaryl, unsubstituted alkylheterocyclyl;
wherein R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;
and wherein R_{12′″} is selected from hydrogen, unsubstituted C₁₋₆ alkyl, unsubstituted C₂₋₆ alkenyl, unsubstituted C₂₋₆ alkynyl and -Boc;

R₃ is selected from hydrogen, substituted or unsubstituted C₁₋₆ alkyl, substituted or unsubstituted C₂₋₆ alkenyl and substituted or unsubstituted C₂₋₆ alkynyl;

R₄ is selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9″}, —SR₉ , —S(O)R₉, S(O)₂R₉, —CN, haloalkyl, haloalkoxy, —C(O)OR₉, —C(O)NR₉R_{9′}, —NR₉S(O)₂NR_{9′}R_{9″} and C(CH₃)₂OR₉;
wherein R₉, R_{9′} and R_{9″} are independently selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl;

wherein, the alkyl, alkenyl or alkynyl, also in alkylaryl, alkyheterocyclyl or alkycycloalkyl, other than those defined in R₁, R₂ or R₇ₐ, if substituted, is substituted with one or more substituent/s selected from —OR₁₃, halogen, —CN, haloalkyl, haloalkoxy and —NR₁₃R_{13′″};
wherein R₁₃ is selected from hydrogen, unsubstituted C₁₋₆ alkyl, unsubstituted C₂₋₆ alkenyl, and unsubstituted C₂₋₆ alkynyl;
and wherein R_{13′″} is selected from hydrogen, unsubstituted C₁₋₈ alkyl, unsubstituted C₂₋₈ alkenyl, unsubstituted C₂₋₈ alkynyl and -Boc;

and wherein, the aryl, heterocyclyl or cycloalkyl, also in alkylaryl, alkyheterocyclyl or alkycycloalkyl, other than those defined in R₁, R₂ or R₇ₐ, if substituted, is substituted with one or more substituent/s selected from halogen, —R₁₄, —OR₁₄, —NO₂, —NR₁₄R_{14′″}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄ , —S(O)R₁₄, S(O)₂R₁₄, —CN, haloalkyl, haloalkoxy, —C(O)OR₁₄, —C(O)NR₁₄R_{14′}, —NR₁₄S(O)₂NR_{14′}R_{14″} and C(CH₃)₂—OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;
and wherein R_{14′″} is selected from hydrogen, unsubstituted C₁₋₆ alkyl, unsubstituted C₂₋₆ alkenyl, unsubstituted C₂₋₆ alkynyl and -Boc;

These preferred compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention has the general Formula (I)

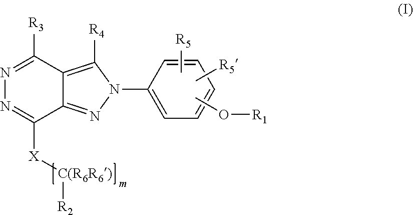

wherein

m is 0, 1, 2, 3, 4 or 5;

X is selected from a bond, —C(RₓR_{x′}, —CH═CH—, —CH₂CH₂—, substituted or unsubstituted cycloalkyl, substituted or unsubstituted aryl and substituted or unsubstituted heterocyclyl;
Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;
R_{x′} is selected from hydrogen, substituted or unsubstituted alkylaryl, substituted or unsubstituted alkylcycloalkyl and substituted or unsubstituted alkylheterocyclyl;
wherein R₈ is selected from hydrogen, substituted or unsubstituted C₁₋₆ alkyl, substituted or unsubstituted C₂₋₆ alkenyl and substituted or unsubstituted C₂₋₆ alkynyl;

R₁ is selected from substituted or unsubstituted C₁₋₆ alkyl, substituted or unsubstituted C₂₋₆ alkenyl and substituted or unsubstituted C₂₋₆ alkynyl;
wherein the alkyl, alkenyl or alkynyl in R₁, if substituted, is substituted with one or more substituent/s selected from —OR₁ , —C(O)R₁₁, halogen, —CN, haloalkyl, haloalkoxy and —NR₁₁R_{11′″};
wherein R₁₁ is selected from hydrogen, unsubstituted C₁₋₆ alkyl, unsubstituted C₂₋₆ alkenyl and unsubstituted C₂₋₆ alkynyl;
and R_{11′″} is selected from hydrogen, unsubstituted C₁₋₆ alkyl, unsubstituted C₂₋₆ alkenyl, unsubstituted C₂₋₆ alkynyl and -Boc;

R₂ is selected from —NR₇R_{7′″}, —CN and substituted or unsubstituted N-containing-heterocyclyl;
wherein said N-containing-heterocyclyl in R₂, if substituted, is mono-substituted with R₇ₐ;
wherein R₇ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc;
and wherein R_{7′″} is selected from hydrogen, substituted or unsubstituted C₁₋₆ alkyl, substituted or unsubstituted C₂₋₆ alkenyl and substituted or unsubstituted C₂₋₆ alkynyl;
wherein R₇ₐ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl and -Boc;
wherein, the alkyl, alkenyl or alkynyl, also in alkylaryl, alkyheterocyclyl or alkycycloalkyl, in R₇ₐ, if substituted, is substituted with one or more substituent/s selected from —OR₁₂, halogen, —CN, haloalkyl, haloalkoxy and —NR₁₂R_{12′″};
and wherein, the aryl, heterocyclyl or cycloalkyl, also in alkylaryl, alkyheterocyclyl or alkycycloalkyl, in R₇ₐ, if substituted, is substituted with one or more substituent/s selected from halogen, —R₁₂, —OR₁₂, —NO₂, —NR₁₂R_{12′″}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —NR₁₂S(O)₂NR_{12′}R_{12″}, unsubstituted alkylcycloalkyl and unsubstituted alkylaryl, unsubstituted alkylheterocyclyl;
wherein R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;
and wherein R_{12′″} is selected from hydrogen, unsubstituted C₁₋₆ alkyl, unsubstituted C₂₋₆ alkenyl, unsubstituted C₂₋₆ alkynyl and -Boc;

R₃ is selected from hydrogen, substituted or unsubstituted C₁₋₆ alkyl, substituted or unsubstituted C₂₋₆ alkenyl and substituted or unsubstituted C₂₋₆ alkynyl; R₄ is selected from hydrogen, substituted or unsubstituted C₁₋₆ alkyl, substituted or unsubstituted C₂₋₆ alkenyl and substituted or unsubstituted C₂₋₆ alkynyl;

R₅ and R_{5′} are independently selected from hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9″}, —SR₉ , —S(O)R₉, S(O)₂R₉, —CN, haloalkyl, haloalkoxy, —C(O)OR₉, —C(O)NR₉R_{9′}, —NR₉S(O)₂NR_{9′}R_{9″} and C(CH₃)₂—OR₉;
wherein R₉, R_{9′} and R_{9″} are independently selected from hydrogen, unsubstituted C₁₋₆ alkyl, unsubstituted C₂₋₆ alkenyl, and unsubstituted C₂₋₆ alkynyl;
and wherein R_{9′″} is selected from hydrogen, unsubstituted C₁₋₈ alkyl, unsubstituted C₂₋₈ alkenyl, unsubstituted C₂₋₈ alkynyl and -Boc;

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl;

wherein, the alkyl, alkenyl or alkynyl, also in alkylaryl, alkyheterocyclyl or alkycycloalkyl, other than those defined in R₁, R₂ or R₇ₐ, if substituted, is substituted with one or more substituent/s selected from —OR₁₃, halogen, —CN, haloalkyl, haloalkoxy and —NR₁₃R_{13′″};
wherein R₁₃ is selected from hydrogen, unsubstituted C₁₋₆ alkyl, unsubstituted C₂₋₆ alkenyl, and unsubstituted C₂₋₆ alkynyl;
and wherein R_{13′″} is selected from hydrogen, unsubstituted C₁₋₈ alkyl, unsubstituted C₂₋₈ alkenyl, unsubstituted C₂₋₈ alkynyl and -Boc;

and wherein, the aryl, heterocyclyl or cycloalkyl, also in alkylaryl, alkyheterocyclyl or alkycycloalkyl, other than those defined in R₁, R₂ or R₇ₐ, if substituted, is substituted with one or more substituent/s selected from halogen, —R₁₄, —OR₁₄, —NO₂, —NR₁₄R_{14′″}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄ , —S(O)R₁₄, S(O)₂R₁₄, —CN, haloalkyl, haloalkoxy, —C(O)OR₁₄, —C(O)NR₁₄R_{14′}, —NR₁₄S(O)₂NR_{14′}R_{14″} and C(CH₃)₂—OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;
and wherein R_{14′″} is selected from hydrogen, unsubstituted C₁₋₆ alkyl, unsubstituted C₂₋₆ alkenyl, unsubstituted C₂₋₆ alkynyl and -Boc;

with the proviso that point of attachment of the —X—[CR₆R_{6′}]ₘ—R₂ moiety to the pyrazolopyridazine structure is not represented by a nitrogen atom.

These preferred compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (In) is a compound wherein

n is 0 or 1;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

m is 0, 1, 2, 3, 4 or 5;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

m is 0, 1, 2 or 3;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (1^{7a}) and (1^{7b}) is a compound wherein

m′ is 1 or 2;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is a bond;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is —C(RₓR_{x′})—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is —CH═CH—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is —CH₂CH₂—;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is substituted or unsubstituted cycloalkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is substituted or unsubstituted aryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

X is substituted or unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is selected from substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₁ is substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₂ is selected from —NR₇R_{7′″}, —CN and substituted or unsubstituted N-containing-heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₃ is selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₃ is selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₃ is substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₄ is selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₄ is selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₄ is substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₅ and R_{5′} are independently selected from hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9″}, —SR₉ , —S(O)R₉, S(O)₂R₉, —CN, haloalkyl, haloalkoxy, —C(O)OR₉, —C(O)NR₉R_{9′}, —NR₉S(O)₂NR_{9′}R_{9″} and C(CH₃)₂—OR₉;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of general Formula (I) is a compound wherein

R₅ and R_{5′} are independently selected from hydrogen and halogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₆ and R_{6′} are independently selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₇ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₇ is selected from hydrogen, substituted or unsubstituted C₁₋₆ alkyl, substituted or unsubstituted alkylaryl, and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₇ is selected from hydrogen, substituted or unsubstituted C₁₋₆ alkyl and substituted or unsubstituted alkylaryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R_{7′″} is selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R_{7′″} is selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₇ₐ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₇ₐ is selected from substituted or unsubstituted C₁₋₆ alkyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₇ₐ is selected from substituted or unsubstituted C₁₋₆ alkyl, substituted or unsubstituted aryl, substituted or unsubstituted alkylcycloalkyl, substituted or unsubstituted alkylaryl, substituted or unsubstituted alkylheterocyclyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

R₇ₐ is selected from substituted or unsubstituted C₁₋₆ alkyl, substituted or unsubstituted aryl, substituted or unsubstituted alkylcycloalkyl, substituted or unsubstituted alkylaryl and substituted or unsubstituted alkylheterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₈ is selected from hydrogen, substituted or unsubstituted C₁₋₆ alkyl, substituted or unsubstituted C₂₋₈ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₈ is selected from hydrogen and substituted or unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein
R₉, R_{9′} and R_{9″} are independently selected from hydrogen, unsubstituted C₁₋₆ alkyl, unsubstituted C₂₋₆ alkenyl, and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₉, R_{9′} and R_{9″} are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{9′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{9′″} is selected from hydrogen, unsubstituted C₁₋₈ alkyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{9′″} is selected from hydrogen and unsubstituted C₁₋₈ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₁ is selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₁ is selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{11′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{11′″} is selected from hydrogen, unsubstituted C₁₋₆ alkyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{11′″} is selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₂, R_{12′} and R_{12″} are independently selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₂, R_{12′} and R_{12″} are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₃ is selected from hydrogen, unsubstituted C₁₋₆ alkyl, unsubstituted C₂₋₆ alkenyl, and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₃ is selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{13′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{13′″} is selected from hydrogen, unsubstituted C₁₋₈ alkyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{13′″} is selected from hydrogen and unsubstituted C₁₋₈ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R₁₄, R_{14′} and R_{14″} are independently selected from hydrogen, unsubstituted C₁₋₆ alkyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{14′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{14′″} is selected from hydrogen, unsubstituted C₁₋₆ alkyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{14′″} is selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

Rₓ is selected from hydrogen, substituted or unsubstituted C₁₋₆ alkyl, —C(O)R₈ and —C(O)OR₈;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

Rₓ is selected from hydrogen and —C(O)OR₈;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

R_{x′} is selected from hydrogen, substituted or unsubstituted alkylaryl, substituted or unsubstituted alkylcycloalkyl and substituted or unsubstituted alkylheterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein R_{x′} is selected from hydrogen and substituted or unsubstituted alkylaryl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the according to the invention of general Formula (I) is a compound wherein

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl;

wherein

Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;

R_{x′} is selected from hydrogen, substituted or unsubstituted alkylaryl, substituted or unsubstituted alkylcycloalkyl and substituted or unsubstituted alkylheterocyclyl;
wherein R₈ is selected from hydrogen, substituted or unsubstituted C₁₋₆ alkyl, substituted or unsubstituted C₂₋₆ alkenyl and substituted or unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

m is 0, 1, 2, 3, 4 or 5; preferably m is 0, 1, 2 or 3;

and/or

R₁ is substituted or unsubstituted C₁₋₆ alkyl; preferably is substituted or unsubstituted ethyl; more preferably unsubstituted ethyl;

and/or

R₂ is selected from —NR₇R_{7′″}, —CN and substituted or unsubstituted N-containing-heterocyclyl; preferably is selected from —NH₂, substituted or unsubstituted —N(H)(methyl), substituted or unsubstituted —N(H)(benzyl), substituted or unsubstituted—N(methyl)(phenethyl), substituted or unsubstituted —N(H)(phenethyl), —N(H)(Boc), substituted or unsubstituted —N(methyl)(Boc), substituted or unsubstituted —N(methyl)₂, —CN, substituted or unsubstituted tetrahydropyridine, substituted or unsubstituted azetidine and substituted or unsubstituted piperidine; more preferably is selected from —NH₂, unsubstituted —N(H)(methyl), unsubstituted —N(H)(benzyl), unsubstituted —N(methyl)(phenethyl), unsubstituted —N(H)(phenethyl), —N(H)(Boc), unsubstituted —N(methyl)(Boc), unsubstituted —N(methyl)₂, —CN, substituted or unsubstituted tetrahydropyridine, substituted or unsubstituted azetidine and substituted or unsubstituted piperidine;

and/or

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl; preferably is selected from a bond, —C(RₓR_{x′})—, —CH═CH—, substituted or unsubstituted aryl and substituted or unsubstituted heterocyclyl; more preferably is selected from a bond, —C(H)(C(O))-ter-butyl)-, —C(benzyl)(C(O)O-ter-butyl)-, —C(H)(benzyl)-, —CH═CH—, substituted or unsubstituted phenyl and substituted or unsubstituted benzimidazol;

and/or

Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈; preferably Rₓ is selected from hydrogen and —C(O)OR₈; more preferably Rₓ is hydrogen or —C(O)O-ter-butyl;

and/or

R_{x′} is selected from hydrogen, substituted or unsubstituted alkylaryl, substituted or unsubstituted alkylcycloalkyl and substituted or unsubstituted alkylheterocyclyl; preferably R_{x′} is selected from hydrogen and substituted or unsubstituted alkylaryl; more preferably R_{x′} is hydrogen or substituted or unsubstituted benzyl;

and/or

R₃ is selected from hydrogen, substituted or unsubstituted C₁₋₆ alkyl, substituted or unsubstituted C₂₋₆ alkenyl and substituted or unsubstituted C₂₋₆ alkynyl; preferably R₃ is substituted or unsubstituted C₁₋₆ alkyl; more preferably R₃ is unsubstituted C₁₋₆ alkyl; even more preferably R₃ is substituted or unsubstituted methyl; even more preferably R₃ is unsubstituted methyl;

and/or

R₄ is selected from hydrogen, substituted or unsubstituted C₁₋₆ alkyl, substituted or unsubstituted C₂₋₆ alkenyl and substituted or unsubstituted C₂₋₆ alkynyl; preferably R₄ is substituted or unsubstituted C₁₋₆ alkyl; more preferably R₄ is unsubstituted C₁₋₆ alkyl; even more preferably R₄ is substituted or unsubstituted methyl; even more preferably R₄ is unsubstituted methyl;

and/or

R₅ and R_{5′} are independently selected from hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9″}, —SR₉ , —S(O)R₉, S(O)₂R₉, —CN, haloalkyl, haloalkoxy, —C(O)OR₉, —C(O)NR₉R_{9′}, —NR₉S(O)₂NR_{9′}R_{9″} and C(CH₃)₂—OR₉; preferably R₅ and R_{5′} are selected from hydrogen and halogen; more preferably R₅ and R_{5′} are selected from hydrogen and fluorine;

and/or

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl; more preferably, R₆ and R_{6′} are both hydrogen;

and/or

R₇ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc; preferably R₇ is selected from hydrogen, substituted or unsubstituted C₁₋₆ alkyl, substituted or unsubstituted alkylaryl and -Boc; more preferably, R₇ is hydrogen, substituted or unsubstituted methyl, substituted or unsubstituted benzyl, substituted or unsubstituted phenethyl and -Boc; even more preferably, R₇ is hydrogen, unsubstituted methyl, unsubstituted benzyl, unsubstituted phenethyl and -Boc;

and/or

R_{7′″} is selected from hydrogen, substituted or unsubstituted C₁₋₆ alkyl, substituted or unsubstituted C₂₋₆ alkenyl and substituted or unsubstituted C₂₋₆ alkynyl; preferably R_{7′″} is selected from hydrogen and substituted or unsubstituted C₁₋₆ alkyl; more preferably, R_{7′″} is hydrogen or substituted or unsubstituted methyl; even more preferably, R_{7′″}is hydrogen or unsubstituted methyl;

and/or

R₇ₐ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl and -Boc; preferably, R₇ₐ is selected from substituted or unsubstituted C₁₋₆ alkyl, substituted or unsubstituted aryl, substituted or unsubstituted alkylcycloalkyl, substituted or unsubstituted alkylaryl, substituted or unsubstituted alkylheterocyclyl and -Boc; more preferably, R₇ₐ is selected from substituted or unsubstituted methyl, substituted or unsubstituted phenyl, substituted or unsubstituted —CH₂-cyclopropyl, substituted or unsubstituted benzyl, substituted or unsubstituted phenethyl, substituted or unsubstituted —CH₂—CH(OH)-phenethyl, substituted or unsubstituted —CH₂-tetrahydropyran, substituted or unsubstituted —CH₂—CH₂-tetrahydropyran, substituted or unsubstituted —CH₂-triazole, substituted or unsubstituted —CH₂—CH₂-pyridine, substituted or unsubstituted CH₂-pyridine and -Boc;

and/or

R₈ is selected from hydrogen, substituted or unsubstituted C₁₋₆ alkyl, substituted or unsubstituted C₂₋₆ alkenyl and substituted or unsubstituted C₂₋₆ alkynyl; preferably R₈ is substituted or unsubstituted C₁₋₆ alkyl; more preferably, R₈ is substituted or unsubstituted ter-butyl; even more preferably, R₈ is unsubstituted ter-butyl;

and/or

R₉, R_{9′} and R_{9″} are independently selected from hydrogen, unsubstituted C₁₋₆ alkyl, unsubstituted C₂₋₆ alkenyl, and unsubstituted C₂₋₆ alkynyl;

and/or

R_{9′″} is selected from hydrogen, unsubstituted C₁₋₈ alkyl, unsubstituted C₂₋₈ alkenyl, unsubstituted C₂₋₈ alkynyl and -Boc;

and/or

R₁₁ is selected from hydrogen, unsubstituted C₁₋₆ alkyl, unsubstituted C₂₋₆ alkenyl and unsubstituted C₂₋₆ alkynyl;

and/or

and R_{11′″} is selected from hydrogen, unsubstituted C₁₋₆ alkyl, unsubstituted C₂₋₆ alkenyl, unsubstituted C₂₋₆ alkynyl and -Boc;

and/or

R₁₂, R_{12′} and R_{12″} are independently selected from hydrogen, unsubstituted C₁₋₆ alkyl, unsubstituted C₂₋₆ alkenyl and unsubstituted C₂₋₆ alkynyl; preferably R₁₂, R_{12′} and R_{12″} are independently selected from hydrogen and unsubstituted C₁₋₆ alkyl; more preferably R₁₂, R_{12′} and R_{12″} are independently selected from hydrogen and unsubstituted methyl;

and/or

and wherein R_{12′″} is selected from hydrogen, unsubstituted C₁₋₆ alkyl, unsubstituted C₂₋₆ alkenyl, unsubstituted C₂₋₆ alkynyl and -Boc;

and/or

R₁₃ is selected from hydrogen, unsubstituted C₁₋₆ alkyl, unsubstituted C₂₋₆ alkenyl, and unsubstituted C₂₋₆ alkynyl;

and/or

R_{13′″} is selected from hydrogen, unsubstituted C₁₋₈ alkyl, unsubstituted C₂₋₈ alkenyl, unsubstituted C₂₋₈ alkynyl and -Boc;

and/or

R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

and/or

R_{14′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention of general Formula (I) is a compound wherein

m is 0, 1, 2, 3, 4 or 5; preferably m is 0, 1, 2 or 3;

and/or

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl; preferably is selected from a bond, —C(RₓR_{x′})—, —CH═CH—, —CH₂CH₂—, substituted or unsubstituted aryl and substituted or unsubstituted heterocyclyl; more preferably is selected from a bond, —C(H)(C(O)O-ter-butyl)-, —C(benzyl)(C(O)O-ter-butyl)-, —C(H)(benzyl)-, —CH═CH—, substituted or unsubstituted phenyl and substituted or unsubstituted benzimidazol;

wherein
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphthyl, or anthracene; preferably is naphthyl and phenyl; preferably the aryl is phenyl;

and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;preferably the heterocyclyl is benzimidazole;

and/or

R₁ is selected from substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl,pentyl, hexyl, isopropyl, or 2-methylpropyl, more preferably the C₁₋₆ alkyl is ethyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or

R₂ is selected from —NR₇R_{7′″}, —CN and substituted or unsubstituted N-containing-heterocyclyl;
wherein
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; preferably, the heterocyclyl is tetrahydropyran, azetidine or piperidine;
and/or

R₃ is selected from hydrogen, substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl,pentyl, hexyl, isopropyl, or 2-methylpropyl, more preferably the C₁₋₆ alkyl is methyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or

R₄ is selected from hydrogen, substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl,pentyl, hexyl, isopropyl, or 2-methylpropyl, more preferably the C₁₋₆ alkyl is methyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or

R₅ and R_{5′} are independently selected from hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9″}, —SR₉ , —S(O)R₉, S(O)₂R₉, —CN, haloalkyl, haloalkoxy, —C(O)OR₉, —C(O)NR₉R_{9′}, —NR₉S(O)₂NR_{9′}R_{9″} and C(CH₃)₂—OR₉;

wherein
the alkyl is C₁₋₆ alkyl like methyl, ethyl, propyl, butyl,pentyl, hexyl, isopropyl, or 2-methylpropyl;
and/or

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or

R₇ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc;

wherein
the C₁₋₆ alkyl is preferably selected from methyl, ethyl, propyl, butyl,pentyl, hexyl, isopropyl, or 2-methylpropyl, more preferably the C₁₋₆ alkyl is methyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or
the alkyl is C₁₋₆ alkyl like methyl, ethyl, propyl, butyl,pentyl, hexyl, isopropyl, or 2-methylpropyl; preferably the alkyl is methyl or ethyl;

and/or

R_{7′″} is selected from hydrogen, substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl,pentyl, hexyl, isopropyl, or 2-methylpropyl, more preferably the C₁₋₆ alkyl is methyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or

R₇ₐ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl and -Boc;
wherein
the C₁₋₆ alkyl is preferably selected from methyl, ethyl, propyl, butyl,pentyl, hexyl, isopropyl, or 2-methylpropyl, more preferably the C₁₋₆ alkyl is methyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or
the alkyl is C₁₋₆ alkyl like methyl, ethyl, propyl, butyl,pentyl, hexyl, isopropyl, or 2-methylpropyl; preferably the alkyl is methyl or ethyl;
and/or
the aryl is selected from phenyl, naphthyl, or anthracene; preferably is naphthyl and phenyl; preferably, the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; preferably the heterocyclyl is tetrahydropyran, triazole or pyridine;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or

R₈ is selected from hydrogen, substituted or unsubstituted C₁₋₆ alkyl, substituted or unsubstituted C₂₋₆ alkenyl and substituted or unsubstituted C₂₋₆ alkynyl;

wherein
the C₁₋₆ alkyl is preferably selected from methyl, ethyl, propyl, butyl,pentyl, hexyl, isopropyl, or 2-methylpropyl; preferably the C₁₋₆ alkyl is ter-butyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or

R₉, R_{9′} and R_{9″} are independently selected from hydrogen, unsubstituted C₁₋₆ alkyl, unsubstituted C₂₋₆ alkenyl, and unsubstituted C₂₋₆ alkynyl;

wherein
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

and/or

R_{9′″} is selected from hydrogen, unsubstituted C₁₋₈ alkyl, unsubstituted C₂₋₈ alkenyl, unsubstituted C₂₋₈ alkynyl and -Boc; wherein
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or

Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;

wherein
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or

R_{x′} is selected from hydrogen, substituted or unsubstituted alkylaryl, substituted or unsubstituted alkylcycloalkyl and substituted or unsubstituted alkylheterocyclyl;

wherein
the alkyl is C₁₋₆ alkyl like methyl, ethyl, propyl, butyl,pentyl, hexyl, isopropyl, or 2-methylpropyl; preferably the alkyl is methyl;
and/or
the aryl is selected from phenyl, naphthyl, or anthracene; preferably is naphthyl and phenyl; preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or

R₁₁ is selected from hydrogen, unsubstituted C₁₋₆ alkyl, unsubstituted C₂₋₆ alkenyl and unsubstituted C₂₋₆ alkynyl; wherein
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or

R_{11′″} is selected from hydrogen, unsubstituted C₁₋₆ alkyl, unsubstituted C₂₋₆ alkenyl, unsubstituted C₂₋₆ alkynyl and -Boc; wherein
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

and/or

R₁₂, R_{12′} and R_{12″} are independently selected from hydrogen, unsubstituted C₁₋₆ alkyl, unsubstituted C₂₋₆ alkenyl and unsubstituted C₂₋₆ alkynyl; wherein
the C₁₋₆ alkyl is preferably selected from methyl, ethyl, propyl, butyl,pentyl, hexyl, isopropyl, or 2-methylpropyl, more preferably the C₁₋₆ alkyl is methyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

and/or

R_{12′″} is selected from hydrogen, unsubstituted C₁₋₆ alkyl, unsubstituted C₂₋₆ alkenyl, unsubstituted C₂₋₆ alkynyl and -Boc; wherein
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or

R₁₃ is selected from hydrogen, unsubstituted C₁₋₆ alkyl, unsubstituted C₂₋₆ alkenyl, and unsubstituted C₂₋₆ alkynyl; wherein
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or

R_{13′″} is selected from hydrogen, unsubstituted C₁₋₈ alkyl, unsubstituted C₂₋₈ alkenyl, unsubstituted C₂₋₈ alkynyl and -Boc; wherein
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

and/or

R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl; wherein
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;

and/or

R_{14′″} is selected from hydrogen, unsubstituted C₁₋₆ alkyl, unsubstituted C₂₋₆ alkenyl, unsubstituted C₂₋₆ alkynyl and -Boc; wherein
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁ as defined in any of the embodiments of the present invention,
the C₁₋₆ alkyl is preferably selected from methyl, ethyl, propyl, butyl,pentyl, hexyl, isopropyl, or 2-methylpropyl, more preferably the C₁₋₆ alkyl is ethyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₂ as defined in any of the embodiments of the present invention,
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; preferably, the heterocyclyl is tetrahydropyran, azetidine or piperidine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in Rₓ as defined in any of the embodiments of the present invention,
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{x′} as defined in any of the embodiments of the present invention,
the alkyl is C₁₋₆ alkyl like methyl, ethyl, propyl, butyl,pentyl, hexyl, isopropyl, or 2-methylpropyl; preferably the alkyl is methyl;
and/or
the aryl is selected from phenyl, naphthyl, or anthracene; preferably is naphthyl and phenyl; preferably the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₃ as defined in any of the embodiments of the present invention,
the C₁₋₆ alkyl is preferably selected from methyl, ethyl, propyl, butyl,pentyl, hexyl, isopropyl, or 2-methylpropyl, more preferably the C₁₋₆ alkyl is methyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₄ as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₅ and R_{5′} as defined in any of the embodiments of the present invention,
the alkyl is C₁₋₆ alkyl like methyl, ethyl, propyl, butyl,pentyl, hexyl, isopropyl, or 2-methylpropyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₆ and R_{6′} as defined in any of the embodiments of the present invention,
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₇ as defined in any of the embodiments of the present invention,
the C₁₋₆ alkyl is preferably selected from methyl, ethyl, propyl, butyl,pentyl, hexyl, isopropyl, or 2-methylpropyl, more preferably the C₁₋₆ alkyl is methyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or
the alkyl is C₁₋₆ alkyl like methyl, ethyl, propyl, butyl,pentyl, hexyl, isopropyl, or 2-methylpropyl; preferably the alkyl is methyl or ethyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{7′″} as defined in any of the embodiments of the present invention,
the C₁₋₆ alkyl is preferably selected from methyl, ethyl, propyl, butyl,pentyl, hexyl, isopropyl, or 2-methylpropyl, more preferably the C₁₋₆ alkyl is methyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₇ₐ as defined in any of the embodiments of the present invention,
the C₁₋₆ alkyl is preferably selected from methyl, ethyl, propyl, butyl,pentyl, hexyl, isopropyl, or 2-methylpropyl, more preferably the C₁₋₆ alkyl is methyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or
the alkyl is C₁₋₆ alkyl like methyl, ethyl, propyl, butyl,pentyl, hexyl, isopropyl, or 2-methylpropyl; preferably the alkyl is methyl or ethyl;
and/or
the aryl is selected from phenyl, naphthyl, or anthracene; preferably is naphthyl and phenyl; preferably, the aryl is phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline; preferably the heterocyclyl is tetrahydropyran, triazole or pyridine;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₈ as defined in any of the embodiments of the present invention,
the C₁₋₆ alkyl is preferably selected from methyl, ethyl, propyl, butyl,pentyl, hexyl, isopropyl, or 2-methylpropyl; preferably the C₁₋₆ alkyl is ter-butyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₉, R_{9′} and R_{9″} as defined in any of the embodiments of the present invention,
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R′″₉ as defined in any of the embodiments of the present invention,
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₁ as defined in any of the embodiments of the present invention,
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{11′} as defined in any of the embodiments of the present invention,
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₂, R_{12′} and R_{12″} as defined in any of the embodiments of the present invention,
the C₁₋₆ alkyl is preferably selected from methyl, ethyl, propyl, butyl,pentyl, hexyl, isopropyl, or 2-methylpropyl, more preferably the C₁₋₆ alkyl is methyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{12′″} as defined in any of the embodiments of the present invention,
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₃ as defined in any of the embodiments of the present invention,
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{13′″} as defined in any of the embodiments of the present invention,
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R₁₄, R_{14′} and R_{14″} as defined in any of the embodiments of the present invention,
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;
and/or
the aryl is selected from phenyl, naphthyl, or anthracene; preferably is naphthyl and phenyl;
and/or
the heterocyclyl is a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyran, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein in R_{14′″} as defined in any of the embodiments of the present invention,
the C₁₋₆ alkyl is preferably selected from methyl, ethyl, propyl, butyl,pentyl, hexyl, isopropyl, or 2-methylpropyl;
and/or
the C₂₋₆ -alkenyl is preferably selected from ethylene, propylene, butylene, pentylene, hexylene, isopropylene and isobutylene;
and/or
the C₂₋₆ -alkynyl is preferably selected from ethyne, propyne, butyne, pentyne, hexyne, isopropyne and isobutyne;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

n is 0 or 1; preferably n is 1;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein m is 0, 1, 2, 3, 4 or 5; preferably m is 0, 1, 2 or 3;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁ is substituted or unsubstituted C₁₋₆ alkyl; preferably is substituted or unsubstituted ethyl; more preferably unsubstituted ethyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₂ is selected from —NR₇R_{7′″}, —CN and substituted or unsubstituted N-containing-heterocyclyl; preferably is selected from —NH₂, substituted or unsubstituted —N(H)(methyl), substituted or unsubstituted —N(H)(benzyl), substituted or unsubstituted—N(methyl)(phenethyl), substituted or unsubstituted —N(H)(phenethyl), —N(H)(Boc), substituted or unsubstituted —N(methyl)(Boc), substituted or unsubstituted —N(methyl)₂, —CN, substituted or unsubstituted tetrahydropyridine, substituted or unsubstituted azetidine and substituted or unsubstituted piperidine; more preferably is selected from —NH₂, unsubstituted —N(H)(methyl), unsubstituted —N(H)(benzyl), unsubstituted —N(methyl)(phenethyl), unsubstituted —N(H)(phenethyl), —N(H)(Boc), unsubstituted —N(methyl)(Boc), unsubstituted —N(methyl)₂, —CN, substituted or unsubstituted tetrahydropyridine, substituted or unsubstituted azetidine and substituted or unsubstituted piperidine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

X is selected from a bond, —C(RₓR_{x′})—, —CH═CH—, —CH₂CH₂—, substituted or unsubstituted cycloalkyl, substituted or unsubstituted aryl and substituted or unsubstituted heterocyclyl; preferably is selected from a bond, —C(RₓR_{x′})—, —CH═CH—, —CH₂CH₂—, substituted or unsubstituted aryl and substituted or unsubstituted heterocyclyl; more preferably is selected from a bond, —C(H)(C(O)O-ter-butyl)-, —C(benzyl)(C(O)O-ter-butyl)-, —C(H)(benzyl)-, —CH═CH—, —CH₂CH₂—, substituted or unsubstituted phenyl and substituted or unsubstituted benzimidazole;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

Rₓ is selected from hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈; preferably is selected from hydrogen and —C(O)OR₈; more preferably Rₓ is hydrogen or —C(O)O-ter-butyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R_{x′} is selected from hydrogen, substituted or unsubstituted alkylaryl, substituted or unsubstituted alkylcycloalkyl and substituted or unsubstituted alkylheterocyclyl; preferably is selected from hydrogen and substituted or unsubstituted alkylaryl; more preferably R_{x′} is hydrogen or substituted or unsubstituted benzyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₃ is selected from hydrogen, substituted or unsubstituted C₁₋₆ alkyl, substituted or unsubstituted C₂₋₆ alkenyl and substituted or unsubstituted C₂₋₆ alkynyl; preferably R₃ is substituted or unsubstituted C₁₋₆ alkyl; more preferably R₃ is unsubstituted C₁₋₆ alkyl; even more preferably R₃ is substituted or unsubstituted methyl; even more preferably R₃ is unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₄ is selected from hydrogen, substituted or unsubstituted C₁₋₆ alkyl, substituted or unsubstituted C₂₋₆ alkenyl and substituted or unsubstituted C₂₋₆ alkynyl; preferably R₄ is substituted or unsubstituted C₁₋₆ alkyl; more preferably R₄ is unsubstituted C₁₋₆ alkyl; even more preferably R₄ is substituted or unsubstituted methyl; even more preferably R₄ is unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₅ and R_{5′} are independently selected from hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9″}, —SR₉ , —S(O)R₉, S(O)₂R₉, —CN, haloalkyl, haloalkoxy, —C(O)OR₉, —C(O)NR₉R_{9′}, —NR₉S(O)₂NR_{9′}R_{9″} and C(CH₃)₂—OR₉; preferably R₅ and R₅ are selected from hydrogen and halogen; more preferably R₅ and R_{5′} are selected from hydrogen and fluorine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl; more preferably, R₆ and R_{6′} are both hydrogen;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₇ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc; preferably R₇ is selected from hydrogen, substituted or unsubstituted C₁₋₆ alkyl, substituted or unsubstituted alkylaryl and -Boc; more preferably, R₇ is hydrogen, substituted or unsubstituted methyl, substituted or unsubstituted benzyl, substituted or unsubstituted phenethyl and -Boc; even more preferably, R₇ is hydrogen, unsubstituted methyl, unsubstituted benzyl, unsubstituted phenethyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R_{7′″} is selected from hydrogen, substituted or unsubstituted C₁₋₆ alkyl, substituted or unsubstituted C₂₋₆ alkenyl and substituted or unsubstituted C₂₋₆ alkynyl; preferably R_{7′″} is selected from hydrogen and substituted or unsubstituted C₁₋₆ alkyl; more preferably, R_{7′″} is hydrogen or substituted or unsubstituted methyl; even more preferably, R_{7′″} is hydrogen or unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₇ₐ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl and -Boc; preferably, R₇ₐ is selected from substituted or unsubstituted C₁₋₆ alkyl, substituted or unsubstituted aryl, substituted or unsubstituted alkylcycloalkyl, substituted or unsubstituted alkylaryl, substituted or unsubstituted alkylheterocyclyl and -Boc; more preferably, R₇ₐ is selected from substituted or unsubstituted methyl, substituted or unsubstituted phenyl, substituted or unsubstituted —CH₂-cyclopropyl, substituted or unsubstituted benzyl, substituted or unsubstituted phenethyl, substituted or unsubstituted —CH₂—CH(OH)-phenethyl, substituted or unsubstituted —CH₂-tetrahydropyran, substituted or unsubstituted —CH₂—CH₂-tetrahydropyran, substituted or unsubstituted —CH₂-triazole, substituted or unsubstituted —CH₂—CH₂-pyridine, substituted or unsubstituted CH₂-pyridine and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₈ is selected from hydrogen, substituted or unsubstituted C₁₋₆ alkyl, substituted or unsubstituted C₂₋₆ alkenyl and substituted or unsubstituted C₂₋₆ alkynyl; preferably R₈ is substituted or unsubstituted C₁₋₆ alkyl; more preferably, R₈ is substituted or unsubstituted ter-butyl; even more preferably, R₈ is unsubstituted ter-butyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₉, R_{9′} and R_{9″} are independently selected from hydrogen, unsubstituted C₁₋₆ alkyl, unsubstituted C₂₋₆ alkenyl, and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R_{9′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁₁ is selected from hydrogen, unsubstituted C₁₋₆ alkyl, unsubstituted C₂₋₆ alkenyl and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

and R_{11′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁₂, R_{12′} and R_{12″} are independently selected from hydrogen, unsubstituted C₁₋₆ alkyl, unsubstituted C₂₋₆ alkenyl and unsubstituted C₂₋₆ alkynyl; preferably R₁₂, R_{12′} and R_{12″} are independently selected from hydrogen and unsubstituted C₁₋₆ alkyl; more preferably R₁₂, R_{12′} and R_{12″} are independently selected from hydrogen and unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R_{12′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁₃ is selected from hydrogen, unsubstituted C₁₋₆ alkyl, unsubstituted C₂₋₆ alkenyl, and unsubstituted C₂₋₆ alkynyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R_{13′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

R_{14′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to general Formula (I) the compound is a compound, wherein

m is 0, 1, 2 or 3;

and

R₁ is substituted or unsubstituted ethyl; more preferably unsubstituted ethyl; and

R₂ is selected from —NH₂, substituted or unsubstituted —N(H)(methyl), substituted or unsubstituted —N(H)(benzyl), substituted or unsubstituted —N(methyl)(phenethyl), substituted or unsubstituted —N(H)(phenethyl), —N(H)(Boc), substituted or unsubstituted —N(methyl)(Boc), substituted or unsubstituted —N(methyl)₂, —CN, substituted or unsubstituted tetrahydropyridine, substituted or unsubstituted azetidine and substituted or unsubstituted piperidine; more preferably is selected from —NH₂, unsubstituted —N(H)(methyl), unsubstituted —N(H)(benzyl), unsubstituted —N(methyl)(phenethyl), unsubstituted —N(H)(phenethyl), —N(H)(Boc), unsubstituted —N(methyl)(Boc), unsubstituted —N(methyl)₂, —CN, substituted or unsubstituted tetrahydropyridine, substituted or unsubstituted azetidine and substituted or unsubstituted piperidine;

and

X is selected from a bond, —C(RₓR_{4′})—, —CH═CH—, —CH₂CH₂—, substituted or unsubstituted aryl and substituted or unsubstituted heterocyclyl; more preferably is selected from a bond, —C(H)(C(O)O-ter-butyl)-, —C(benzyl)(C(O)O-ter-butyl)-, —C(H)(benzyl)-, —CH═CH—, substituted or unsubstituted phenyl and substituted or unsubstituted benzimidazole;

and

R_{x′} is selected from hydrogen and —C(O)OR₈; more preferably Rₓ is hydrogen or —C(O)O-ter-butyl;

and

R_{x′} is selected from hydrogen and substituted or unsubstituted alkylaryl; more preferably R_{x′} is hydrogen or substituted or unsubstituted benzyl;

and

R₃ is substituted or unsubstituted C₁₋₆ alkyl; more preferably R₃ is unsubstituted C₁₋₆ alkyl; even more preferably R₃ is substituted or unsubstituted methyl; even more preferably R₃ is unsubstituted methyl;

and

R₄ is substituted or unsubstituted C₁₋₆ alkyl; more preferably R₄ is unsubstituted C₁₋₆ alkyl; even more preferably R₄ is substituted or unsubstituted methyl; even more preferably R₄ is unsubstituted methyl;

and

R₅ and R_{5′} are selected from hydrogen and halogen; more preferably R₅ and R_{5′} are selected from hydrogen and fluorine;

and

R₆ and R_{6′} are both hydrogen;

and

R₇ is selected from hydrogen, substituted or unsubstituted C₁₋₆ alkyl, substituted or unsubstituted alkylaryl and -Boc; more preferably, R₇ is hydrogen, substituted or unsubstituted methyl, substituted or unsubstituted benzyl, substituted or unsubstituted phenethyl and -Boc; even more preferably, R₇ is hydrogen, unsubstituted methyl, unsubstituted benzyl, unsubstituted phenethyl and -Boc;

and

R_{7′″} is selected from hydrogen and substituted or unsubstituted C₁₋₆ alkyl; more preferably, R_{7′″} is hydrogen or substituted or unsubstituted methyl; even more preferably, R_{7′″} is hydrogen or unsubstituted methyl;

and

R₇ₐ is selected from substituted or unsubstituted C₁₋₆ alkyl, substituted or unsubstituted aryl, substituted or unsubstituted alkylcycloalkyl, substituted or unsubstituted alkylaryl, substituted or unsubstituted alkylheterocyclyl and -Boc; more preferably, R₇ₐ is selected from substituted or unsubstituted methyl, substituted or unsubstituted phenyl, substituted or unsubstituted —CH₂-cyclopropyl, substituted or unsubstituted benzyl, substituted or unsubstituted phenethyl, substituted or unsubstituted —CH₂—CH(OH)-phenethyl, substituted or unsubstituted —CH₂-tetrahydropyran, substituted or unsubstituted —CH₂—CH₂-tetrahydropyran, substituted or unsubstituted —CH₂-triazole, substituted or unsubstituted —CH₂—CH₂-pyridine, substituted or unsubstituted CH₂-pyridine and -Boc;

and

R₈ is substituted or unsubstituted ter-butyl; even more preferably, R₈ is unsubstituted ter-butyl;

and

R₁₂, R_{12′} and R_{12″} are independently selected from hydrogen and unsubstituted C₁₋₆ alkyl; more preferably R₁₂, R_{12′} and R_{12″} are independently selected from hydrogen and unsubstituted methyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment

n is 1.

In a preferred embodiment

m is 0, 1, 2 or 3.

In a preferred embodiment

R₁ is substituted or unsubstituted ethyl; more preferably unsubstituted ethyl.

In a preferred embodiment

R₂ is selected from —NH₂, substituted or unsubstituted —N(H)(methyl), substituted or unsubstituted —N(H)(benzyl), substituted or unsubstituted —N(methyl)(phenethyl), substituted or unsubstituted —N(H)(phenethyl), —N(H)(Boc), substituted or unsubstituted —N(methyl)(Boc), substituted or unsubstituted —N(methyl)₂, —CN, substituted or unsubstituted tetrahydropyridine, substituted or unsubstituted azetidine and substituted or unsubstituted piperidine; more preferably is selected from —NH₂, unsubstituted —N(H)(methyl), unsubstituted —N(H)(benzyl), unsubstituted —N(methyl)(phenethyl), unsubstituted —N(H)(phenethyl), —N(H)(Boc), unsubstituted —N(methyl)(Boc), unsubstituted —N(methyl)₂, —CN, substituted or unsubstituted tetrahydropyridine, substituted or unsubstituted azetidine and substituted or unsubstituted piperidine.

In a preferred embodiment

R₂ is substituted or unsubstituted group selected from

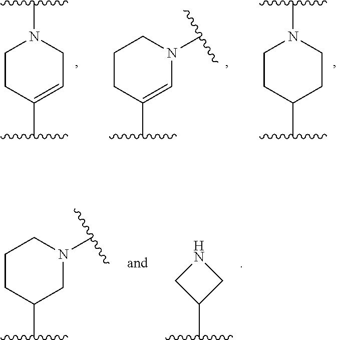 [SMILES: *C1=CCN(*)CC1 ; *C1=CN(*)CCC1 ; *C1CCN(*)CC1 ; *C1CCCN(*)C1 ; *C1CNC1]

In a preferred embodiment

R₂ is unsubstituted group selected from

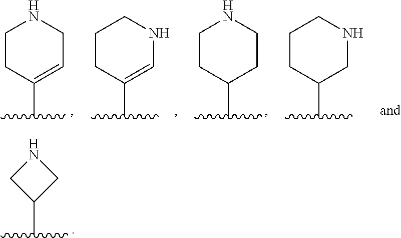

In a preferred embodiment

R₂ is substituted with R₇ₐ and is selected from

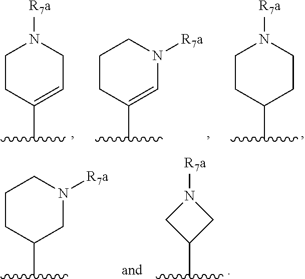

In a preferred embodiment

X is selected from a bond, —C(RₓR_{x′})—, —CH═CH—, —CH₂CH₂—, substituted or unsubstituted aryl and substituted or unsubstituted heterocyclyl; more preferably is selected from a bond, substituted or unsubstituted methyl, substituted or unsubstituted ethyl, substituted or unsubstituted propyl, —C(H)(C(O)O-ter-butyl)-, —C(benzyl)(C(O)O-ter-butyl)-, —C(H)(benzyl)-, —CH═CH—, substituted or unsubstituted phenyl and substituted or unsubstituted benzimidazole.

In a preferred embodiment

X is a substituted or unsubstituted benzimidazole represented by

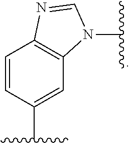 [SMILES: *c1ccc2ncn(*)c2c1]

In a preferred embodiment —X—[C(R₆R_{6′})]ₘ—R₂ is represented by

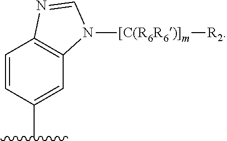

In a preferred embodiment

Rₓ is selected from hydrogen and —C(O)OR₈; more preferably Rₓ is hydrogen or —C(O)O-ter-butyl.

In a preferred embodiment

R_{x′} is selected from hydrogen and substituted or unsubstituted alkylaryl; more preferably R_{x′} is hydrogen or substituted or unsubstituted benzyl.

In a preferred embodiment

Rₓ is hydrogen or substituted or unsubstituted —C(O)O-ter-butyl, preferably hydrogen or unsubstituted —C(O)O-ter-butyl, while R′ₓ is hydrogen or substituted or unsubstituted benzyl, preferably hydrogen or unsubstituted benzyl.

In a preferred embodiment

Rₓ is hydrogen, while R′ₓ is hydrogen or substituted or unsubstituted benzyl, preferably unsubstituted benzyl.

In a preferred embodiment

Rₓ is substituted or unsubstituted —C(O)O-ter-butyl, preferably unsubstituted —C(O)O-ter-butyl, while R′ₓ is hydrogen or substituted or unsubstituted benzyl, preferably hydrogen or unsubstituted benzyl.

In a preferred embodiment

Rₓ is substituted or unsubstituted —C(O)O-ter-butyl, preferably unsubstituted—C(O)O-ter-butyl, while R′ₓ is hydrogen.

In a preferred embodiment

Rₓ is substituted or unsubstituted —C(O)O-ter-butyl, preferably unsubstituted —C(O)O-ter-butyl, while R′ₓ is substituted or unsubstituted benzyl, preferably unsubstituted benzyl.

Rₓ is hydrogen, while R′ₓ is substituted or unsubstituted benzyl, preferably unsubstituted benzyl.

In a preferred embodiment

R_{X} and R′ₓ are both hydrogen.

In a preferred embodiment

R₃ is substituted or unsubstituted C₁₋₆ alkyl; more preferably R₃ is unsubstituted C₁₋₆ alkyl; even more preferably R₃ is substituted or unsubstituted methyl; even more preferably R₃ is unsubstituted methyl.

In a preferred embodiment

R₄ is substituted or unsubstituted C₁₋₆ alkyl; more preferably R₄ is unsubstituted C₁₋₆ alkyl; even more preferably R₄ is substituted or unsubstituted methyl; even more preferably R₄ is unsubstituted methyl.

In a preferred embodiment

R₅ is selected from hydrogen and halogen; preferably R₅ is selected from hydrogen and fluorine; preferably R₅ is selected from hydrogen and fluorine in ortho position.

In a preferred embodiment

R₅ is fluorine; preferably R₅ is fluorine in ortho position.

In a preferred embodiment

R_{5′} is hydrogen.

In a preferred embodiment

R₅ is selected from hydrogen and halogen; preferably R₅ is selected from hydrogen and fluorine; preferably R₅ is selected from hydrogen and fluorine in ortho position, while R_{5′} is hydrogen.

In a preferred embodiment

R₅ is fluorine; preferably R₅ is fluorine in ortho position, while R_{5′} is hydrogen.

In a preferred embodiment

R₆ is hydrogen.

In a preferred embodiment

R_{6′} is hydrogen.

In a preferred embodiment

R₆ and R_{6′} are both hydrogen.

In a preferred embodiment

R₇ is selected from hydrogen, substituted or unsubstituted C₁₋₆ alkyl, substituted or unsubstituted alkylaryl and -Boc; more preferably, R₇ is hydrogen, substituted or unsubstituted methyl, substituted or unsubstituted benzyl, substituted or unsubstituted phenethyl and -Boc; even more preferably, R₇ is hydrogen, unsubstituted methyl, unsubstituted benzyl, unsubstituted phenethyl and -Boc.

In a preferred embodiment

R_{7′″} is selected from hydrogen and substituted or unsubstituted C₁₋₆ alkyl; more preferably, R_{7′″} is hydrogen or substituted or unsubstituted methyl; even more preferably, R_{7′″} is hydrogen or unsubstituted methyl.

In a preferred embodiment

R₇ is selected from hydrogen, substituted or unsubstituted C₁₋₆ alkyl, substituted or unsubstituted alkylaryl and -Boc; more preferably, R₇ is hydrogen, substituted or unsubstituted methyl, substituted or unsubstituted benzyl, substituted or unsubstituted phenethyl and -Boc; even more preferably, R₇ is hydrogen, unsubstituted methyl, unsubstituted benzyl, unsubstituted phenethyl and -Boc, while R_{7′″} is selected from hydrogen and substituted or unsubstituted C₁₋₆ alkyl; more preferably, R_{7′″} is hydrogen or substituted or unsubstituted methyl; even more preferably, R_{7′″} is hydrogen or unsubstituted methyl.

In a preferred embodiment

R₇ is substituted or unsubstituted C₁₋₆ alkyl; more preferably, R₇ is substituted or unsubstituted methyl; even more preferably, R₇ is unsubstituted methyl, while R_{7′″} is selected from hydrogen and substituted or unsubstituted C₁₋₆ alkyl; more preferably, R_{7′″} is hydrogen or substituted or unsubstituted methyl; even more preferably, R_{7′″} is hydrogen or unsubstituted methyl.

In a preferred embodiment

R₇ is -Boc, while R_{7′″} is selected from hydrogen and substituted or unsubstituted C₁₋₆ alkyl; more preferably, R_{7′″} is hydrogen or substituted or unsubstituted methyl; even more preferably, R_{7′″} is hydrogen or unsubstituted methyl.

In a preferred embodiment

R₇ is substituted or unsubstituted alkylaryl; more preferably, R₇ is substituted or unsubstituted benzyl; even more preferably, R₇ is unsubstituted benzyl, while R_{7′″} is selected from hydrogen.

In a preferred embodiment

R₇ is substituted or unsubstituted alkylaryl; more preferably, R₇ is substituted or unsubstituted phenethyl; even more preferably, R₇ is unsubstituted phenethyl, while R_{7′″} is selected from hydrogen and substituted or unsubstituted C₁₋₆ alkyl; more preferably, R_{7′″} is hydrogen or substituted or unsubstituted methyl; even more preferably, R_{7′″} is hydrogen or unsubstituted methyl.

In a preferred embodiment

R₇ and R_{7′″} are both hydrogen.

In a preferred embodiment

R₇ₐ is selected from substituted or unsubstituted C₁₋₆ alkyl, substituted or unsubstituted aryl, substituted or unsubstituted alkylcycloalkyl, substituted or unsubstituted alkylaryl, substituted or unsubstituted alkylheterocyclyl and -Boc; more preferably, R₇ₐ is selected from substituted or unsubstituted methyl, substituted or unsubstituted phenyl, substituted or unsubstituted —CH₂-cyclopropyl, substituted or unsubstituted benzyl, substituted or unsubstituted phenethyl, substituted or unsubstituted —CH₂—CH(OH)-phenethyl, substituted or unsubstituted —CH₂-tetrahydropyran, substituted or unsubstituted —CH₂—CH₂-tetrahydropyran, substituted or unsubstituted —CH₂-triazole, substituted or unsubstituted —CH₂—CH₂-pyridine, substituted or unsubstituted CH₂-pyridine and -Boc.

In a preferred embodiment

R₇ₐ is selected from substituted or unsubstituted —CH₂-tetrahydropyran, substituted or unsubstituted —CH₂-triazole and substituted or unsubstituted CH₂-pyridine , wherein the tetrahydropyran moiety is

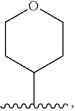 [SMILES: *C1CCOCC1]

the triazole moiety is

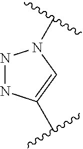 [SMILES: *C1=CN(*)NN1]

and the pyridine moiety is selected from

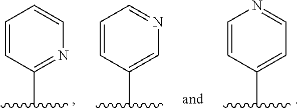

In a preferred embodiment

the aryl, heterocyclyl or cycloalkyl, also in alkylaryl, alkyheterocyclyl or alkycycloalkyl, in R₇ₐ, if substituted, is substituted with one or more substituent/s selected from halogen, —R₁₂, —OR₁₂, —NO₂, —NR₁₂R_{12′″}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —NR₁₂S(O)₂NR_{12′}R_{12″}, unsubstituted alkylcycloalkyl, unsubstituted alkylaryl, and substituted alkylheterocyclyl;

wherein the alkylheterocyclyl is

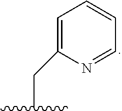 [SMILES: *Cc1ccccn1]

In a preferred embodiment

R₇ₐ substituted with alkylheterocyclyl is represented by

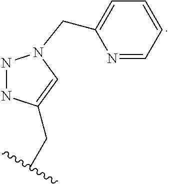 [SMILES: *CC1=CN(Cc2ccccn2)NN1]

In a preferred embodiment

The substituent on R₂ is

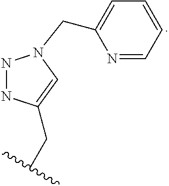 [SMILES: *CC1=CN(Cc2ccccn2)NN1]

In a preferred embodiment

R₈ is substituted or unsubstituted ter-butyl; even more preferably, R₈ is unsubstituted ter-butyl.

In a preferred embodiment

R₁₂, R_{12′} and R_{12″} are independently selected from hydrogen and unsubstituted C₁₋₆ alkyl; more preferably R₁₂, R_{12′} and R_{12″} are independently selected from hydrogen and unsubstituted methyl.

In another preferred embodiment

m is 0.

In another preferred embodiment

m is 1.

In another preferred embodiment

m is 2.

In another preferred embodiment

m is 3.

In another preferred embodiment

X is a bond

In another preferred embodiment

X is —C(RₓR_{x′})—.

In another preferred embodiment

X is —CH═CH—.

In another preferred embodiment

X is —CH₂CH₂—.

In another preferred embodiment

X is substituted or unsubstituted aryl.

In another preferred embodiment

X is substituted or unsubstituted heterocyclyl.

In an particular embodiment

the halogen is fluorine, chlorine, iodine or bromine.

In an particular embodiment

the halogen is fluorine.

In a preferred further embodiment, the compounds of the general Formula (I) are selected from

| Ex | Structure | Name |
|---|---|---|
| 1 | 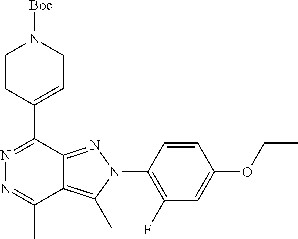 [SMILES: CCOc1ccc(-n2nc3c(C4=CCN(C(=O)OC(C)(C)C)CC4)nnc(C)c3c2C)c(F)c1] | tert-Butyl 4-(2-(4-ethoxy-2-fluorophenyl)-3,4-dimethyl- 2H-pyrazolo[3,4-d]pyridazin-7-yl)-3,6-dihydropyridine- 1(2H)-carboxylate |
| 2 | 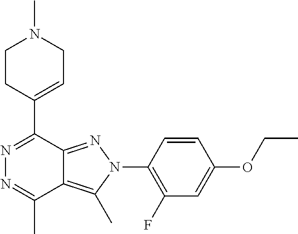 [SMILES: CCOc1ccc(-n2nc3c(C4=CCN(C)CC4)nnc(C)c3c2C)c(F)c1] | 2-(4-Ethoxy-2-fluorophenyl)-3,4-dimethyl-7-(1-methyl- 1,2,3,6-tetrahydropyridin-4-yl)-2H-pyrazolo[3,4- d]pyridazine |
| 3 | 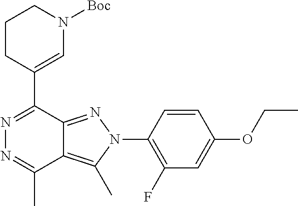 [SMILES: CCOc1ccc(-n2nc3c(C4=CN(C(=O)OC(C)(C)C)CCC4)nnc(C)c3c2C)c(F)c1] | tert-Butyl 5-(2-(4-ethoxy-2-fluorophenyl)-3,4-dimethyl- 2H-pyrazolo[3,4-d]pyridazin-7-yl)-3,4-dihydropyridine- 1(2H)-carboxylate |
| 4 | 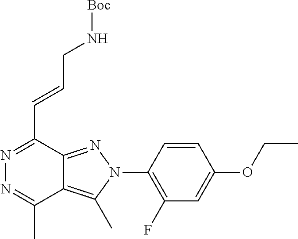 [SMILES: CCOc1ccc(-n2nc3c(/C=C/CNC(=O)OC(C)(C)C)nnc(C)c3c2C)c(F)c1] | tert-Butyl (E)-(3-(2-(4-ethoxy-2-fluorophenyl)-3,4- dimethyl-2H-pyrazolo[3,4-d]pyridazin-7- yl)allyl)carbamate |
| 5 | 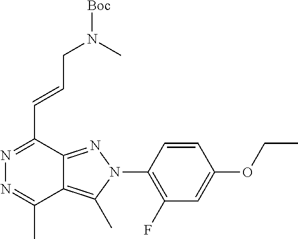 [SMILES: CCOc1ccc(-n2nc3c(/C=C/CN(C)C(=O)OC(C)(C)C)nnc(C)c3c2C)c(F)c1] | tert-Butyl (E)-(3-(2-(4-ethoxy-2-fluorophenyl)-3,4- dimethyl-2H-pyrazolo[3,4-d]pyridazin-7- yl)allyl)(methyl)carbamate |
| 6 | 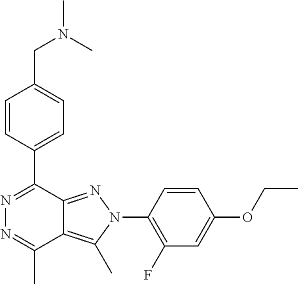 [SMILES: CCOc1ccc(-n2nc3c(-c4ccc(CN(C)C)cc4)nnc(C)c3c2C)c(F)c1] | 1-(4-(2-(4-Ethoxy-2-fluorophenyl)-3,4-dimethyl-2H- pyrazolo[3,4-d]pyridazin-7-yl)phenyl)-N,N- dimethylmethanamine |
| 7 | 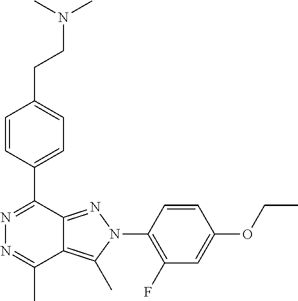 [SMILES: CCOc1ccc(-n2nc3c(-c4ccc(CCN(C)C)cc4)nnc(C)c3c2C)c(F)c1] | 2-(4-(2-(4-Ethoxy-2-fluorophenyl)-3,4-dimethyl-2H- pyrazolo[3,4-d]pyridazin-7-yl)phenyl)-N,N-dimethylethan- 1-amine |
| 8 | 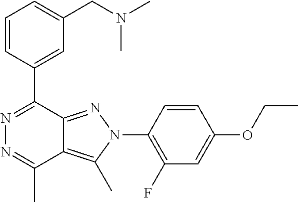 [SMILES: CCOc1ccc(-n2nc3c(-c4cccc(CN(C)C)c4)nnc(C)c3c2C)c(F)c1] | 1-(3-(2-(4-Ethoxy-2-fluorophenyl)-3,4-dimethyl-2H- pyrazolo[3,4-d]pyridazin-7-yl)phenyl)-N,N- dimethylmethanamine |
| 9 | 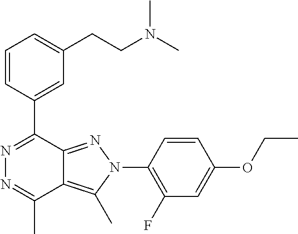 [SMILES: CCOc1ccc(-n2nc3c(-c4cccc(CCN(C)C)c4)nnc(C)c3c2C)c(F)c1] | 2-(3-(2-(4-Ethoxy-2-fluorophenyl)-3,4-dimethyl-2H- pyrazolo[3,4-d]pyridazin-7-yl)phenyl)-N,N-dimethylethan- 1-amine |
| 10 | 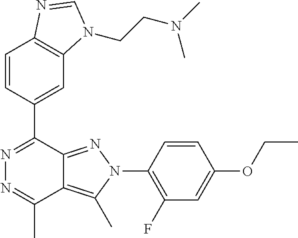 [SMILES: CCOc1ccc(-n2nc3c(-c4ccc5ncn(CCN(C)C)c5c4)nnc(C)c3c2C)c(F)c1] | 2-(6-(2-(4-Ethoxy-2-fluorophenyl)-3,4-dimethyl-2H- pyrazolo[3,4-d]pyridazin-7-yl)-1H-benzo[d]imidazol-1-yl)- N,N-dimethylethan-1-amine |
| 11 | 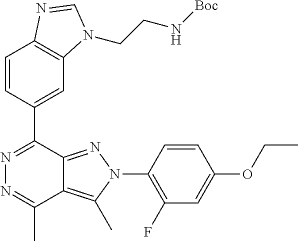 [SMILES: CCOc1ccc(-n2nc3c(-c4ccc5ncn(CCNC(=O)OC(C)(C)C)c5c4)nnc(C)c3c2C)c(F)c1] | tert-butyl (2-(6-(2-(4-ethoxy-2-fluorophenyl)-3,4-dimethyl- 2H-pyrazolo[3,4-d]pyridazin-7-yl)-1H-benzo[d]imidazol- 1-yl)ethyl)carbamate |
| 12 | 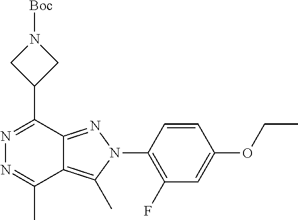 [SMILES: CCOc1ccc(-n2nc3c(C4CN(C(=O)OC(C)(C)C)C4)nnc(C)c3c2C)c(F)c1] | tert-Butyl 3-(2-(4-ethoxy-2-fluorophenyl)-3,4-dimethyl- 2H-pyrazolo[3,4-d]pyridazin-7-yl)azetidine-1-carboxylate |
| 13 | 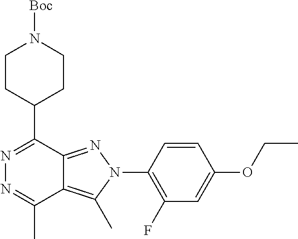 [SMILES: CCOc1ccc(-n2nc3c(C4CCN(C(=O)OC(C)(C)C)CC4)nnc(C)c3c2C)c(F)c1] | tert-Butyl 4-(2-(4-ethoxy-2-fluorophenyl)-3,4-dimethyl- 2H-pyrazolo[3,4-d]pyridazin-7-yl)piperidine-1- carboxylate |
| 14 | 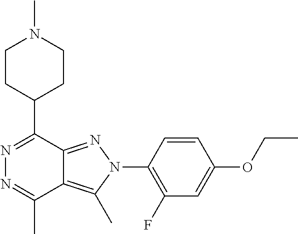 [SMILES: CCOc1ccc(-n2nc3c(C4CCN(C)CC4)nnc(C)c3c2C)c(F)c1] | 2-(4-Ethoxy-2-fluorophenyl)-3,4-dimethyl-7-(1- methylpiperidin-4-yl)-2H-pyrazolo[3,4-d]pyridazine |
| 15 | 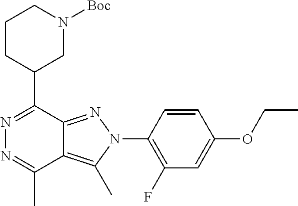 [SMILES: CCOc1ccc(-n2nc3c(C4CCCN(C(=O)OC(C)(C)C)C4)nnc(C)c3c2C)c(F)c1] | tert-Butyl-3-(2-(4-ethoxy-2-fluorophenyl)-3,4-dimethyl- 2H-pyrazolo[3,4-d]pyridazin-7-yl)piperidine-1- carboxylate |
| 16 | 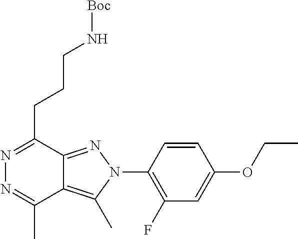 [SMILES: CCOc1ccc(-n2nc3c(CCCNC(=O)OC(C)(C)C)nnc(C)c3c2C)c(F)c1] | tert-Butyl-(3-(2-(4-ethoxy-2-fluorophenyl)-3,4-dimethyl- 2H-pyrazolo[3,4-d]pyridazin-7-yl)propyl)carbamate |
| 17 | 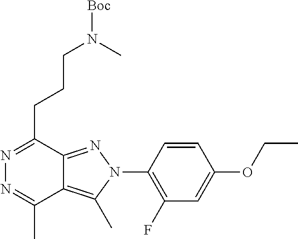 [SMILES: CCOc1ccc(-n2nc3c(CCCN(C)C(=O)OC(C)(C)C)nnc(C)c3c2C)c(F)c1] | tert-Butyl-(3-(2-(4-ethoxy-2-fluorophenyl)-3,4-dimethyl- 2H-pyrazolo[3,4-d]pyridazin-7- yl)propyl)(methyl)carbamate |
| 18 | 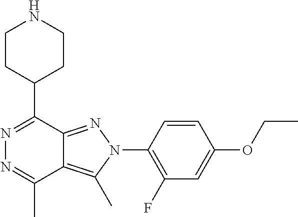 [SMILES: CCOc1ccc(-n2nc3c(C4CCNCC4)nnc(C)c3c2C)c(F)c1] | 2-(4-Ethoxy-2-fluorophenyl)-3,4-dimethyl-7-(piperidin-4- yl)-2H-pyrazolo[3,4-d]pyridazine |
| 19 | 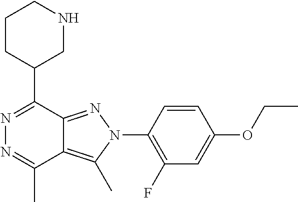 [SMILES: CCOc1ccc(-n2nc3c(C4CCCNC4)nnc(C)c3c2C)c(F)c1] | 2-(4-Ethoxy-2-fluorophenyl)-3,4-dimethyl-7-(piperidin-3- yl)-2H-pyrazolo[3,4-d]pyridazine |
| 20 | 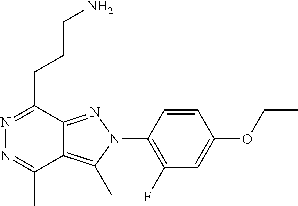 [SMILES: CCOc1ccc(-n2nc3c(CCCN)nnc(C)c3c2C)c(F)c1] | 3-(2-(4-Ethoxy-2-fluorophenyl)-3,4-dimethyl-2H- pyrazolo[3,4-d]pyridazin-7-yl)propan-1-amine |
| 21 | 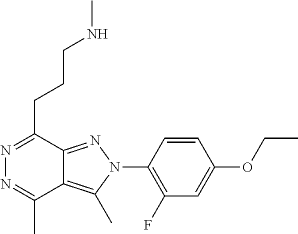 [SMILES: CCOc1ccc(-n2nc3c(CCCNC)nnc(C)c3c2C)c(F)c1] | 3-(2-(4-Ethoxy-2-fluorophenyl)-3,4-dimethyl-2H- pyrazolo[3,4-d]pyridazin-7-yl)-N-methylpropan-1-amine |
| 22 | 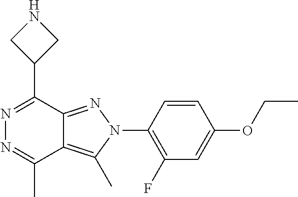 [SMILES: CCOc1ccc(-n2nc3c(C4CNC4)nnc(C)c3c2C)c(F)c1] | 7-(Azetidin-3-yl)-2-(4-ethoxy-2-fluorophenyl)-3,4- dimethyl-2H-pyrazolo[3,4-d]pyridazine |
| 23 | 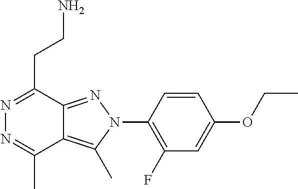 [SMILES: CCOc1ccc(-n2nc3c(CCN)nnc(C)c3c2C)c(F)c1] | 2-(2-(4-Ethoxy-2-fluorophenyl)-3,4-dimethyl-2H- pyrazolo[3,4-d]pyridazin-7-yl)ethan-1-amine |
| 24 | 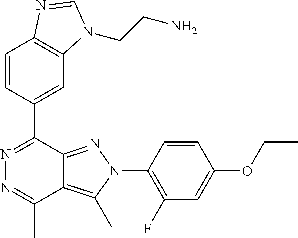 [SMILES: CCOc1ccc(-n2nc3c(-c4ccc5ncn(CCN)c5c4)nnc(C)c3c2C)c(F)c1] | 2-(6-(2-(4-Ethoxy-2-fluorophenyl)-3,4-dimethyl-2H- pyrazolo[3,4-d]pyridazin-7-yl)-1H-benzo[d]imidazol-1- yl)ethan-1-amine |
| 25 | 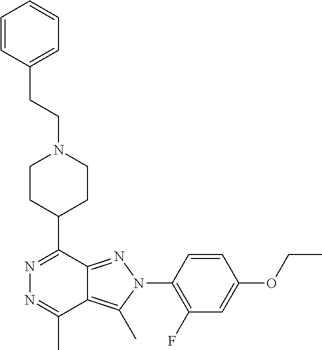 [SMILES: CCOc1ccc(-n2nc3c(C4CCN(CCc5ccccc5)CC4)nnc(C)c3c2C)c(F)c1] | 2-(4-Ethoxy-2-fluorophenyl)-3,4-dimethyl-7-(1- phenethylpiperidin-4-yl)-2H-pyrazolo[3,4-d]pyridazine |
| 26 | 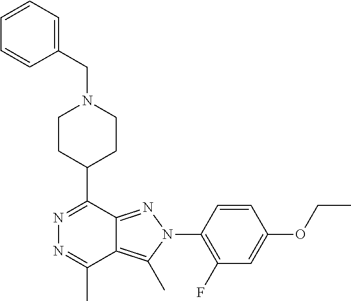 [SMILES: CCOc1ccc(-n2nc3c(C4CCN(Cc5ccccc5)CC4)nnc(C)c3c2C)c(F)c1] | 7-(1-Benzylpiperidin-4-yl)-2-(4-ethoxy-2-fluorophenyl)- 3,4-dimethyl-2H-pyrazolo[3,4-d]pyridazine |
| 27 | 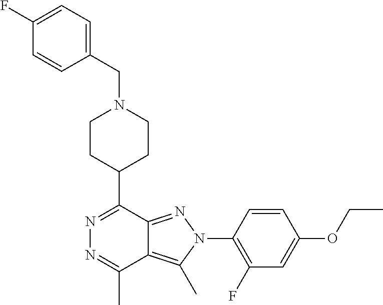 [SMILES: CCOc1ccc(-n2nc3c(C4CCN(Cc5ccc(F)cc5)CC4)nnc(C)c3c2C)c(F)c1] | 2-(4-Ethoxy-2-fluorophenyl)-7-(1-(4- fluorobenzyl)piperidin-4-yl)-3,4-dimethyl-2H- pyrazolo[3,4-d]pyridazine |
| 28 | 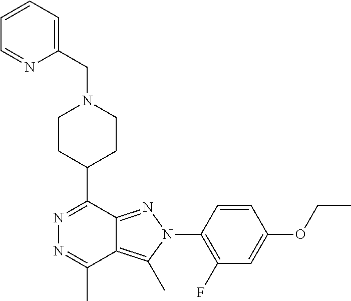 [SMILES: CCOc1ccc(-n2nc3c(C4CCN(Cc5ccccn5)CC4)nnc(C)c3c2C)c(F)c1] | 2-(4-Ethoxy-2-fluorophenyl)-3,4-dimethyl-7-(1-(pyridin-2- ylmethyl)piperidin-4-yl)-2H-pyrazolo[3,4-d]pyridazine |
| 29 | 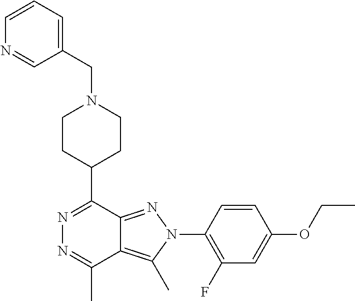 [SMILES: CCOc1ccc(-n2nc3c(C4CCN(Cc5cccnc5)CC4)nnc(C)c3c2C)c(F)c1] | 2-(4-Ethoxy-2-fluorophenyl)-3,4-dimethyl-7-(1-(pyridin-3- ylmethyl)piperidin-4-yl)-2H-pyrazolo[3,4-d]pyridazine |
| 30 | 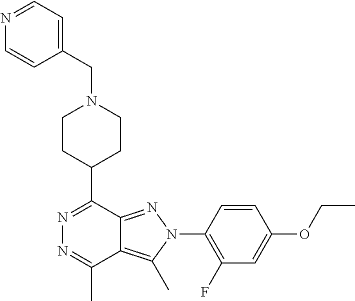 [SMILES: CCOc1ccc(-n2nc3c(C4CCN(Cc5ccncc5)CC4)nnc(C)c3c2C)c(F)c1] | 2-(4-Ethoxy-2-fluorophenyl)-3,4-dimethyl-7-(1-(pyridin-4- ylmethyl)piperidin-4-yl)-2H-pyrazolo[3,4-d]pyridazine |
| 31 | 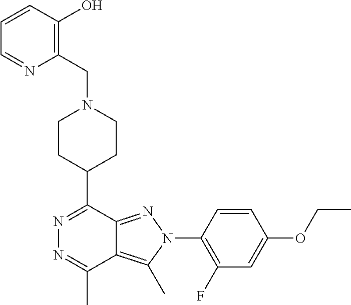 [SMILES: CCOc1ccc(-n2nc3c(C4CCN(Cc5ncccc5O)CC4)nnc(C)c3c2C)c(F)c1] | 2-((4-(2-(4-Ethoxy-2-fluorophenyl)-3,4-dimethyl-2H- pyrazolo[3,4-d]pyridazin-7-yl)piperidin-1- yl)methyl)pyridin-3-ol |
| 32 | 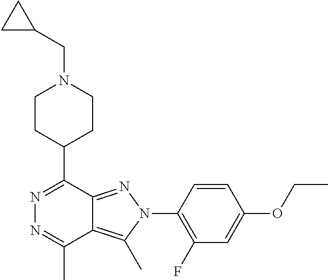 [SMILES: CCOc1ccc(-n2nc3c(C4CCN(CC5CC5)CC4)nnc(C)c3c2C)c(F)c1] | 7-(1-(Cyclopropyl-methyl)piperidin-4-yl)-2-(4-ethoxy-2- fluorophenyl)-3,4-dimethyl-2H-pyrazolo[3,4-d]pyridazine |
| 33 | 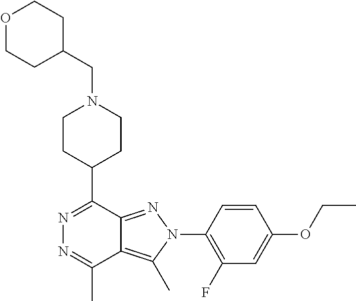 [SMILES: CCOc1ccc(-n2nc3c(C4CCN(CC5CCOCC5)CC4)nnc(C)c3c2C)c(F)c1] | 2-(4-Ethoxy-2-fluorophenyl)-3,4-dimethyl-7-(1- ((tetrahydro-2H-pyran-4-yl)methyl)piperidin-4-yl)-2H- pyrazolo[3,4-d]pyridazine |
| 34 | 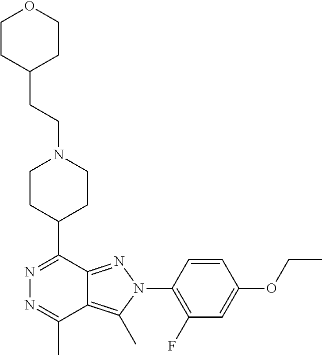 [SMILES: CCOc1ccc(-n2nc3c(C4CCN(CCC5CCOCC5)CC4)nnc(C)c3c2C)c(F)c1] | 2-(4-Ethoxy-2-fluorophenyl)-3,4-dimethyl-7-(1-(2- (tetrahydro-2H-pyran-4-yl)ethyl)piperidin-4-yl)-2H- pyrazolo[3,4-d]pyridazine |
| 35 | 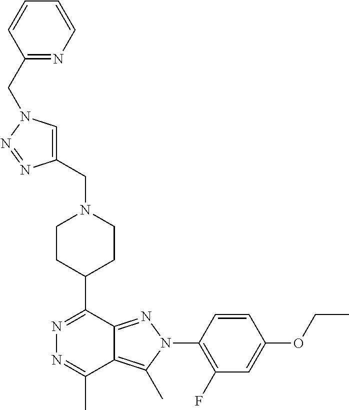 [SMILES: CCOc1ccc(-n2nc3c(C4CCN(Cc5cn(Cc6ccccn6)nn5)CC4)nnc(C)c3c2C)c(F)c1] | 2-(4-Ethoxy-2-fluorophenyl)-3,4-dimethyl-7-(1-((1- (pyridin-2-ylmethyl)-1H-1,2,3-triazol-4- yl)methyl)piperidin-4-yl)-2H-pyrazolo[3,4-d]pyridazine |
| 36 | 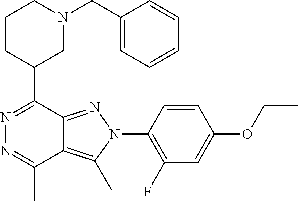 [SMILES: CCOc1ccc(-n2nc3c(C4CCCN(Cc5ccccc5)C4)nnc(C)c3c2C)c(F)c1] | 7-(1-Benzylpiperidin-3-yl)-2-(4-ethoxy-2-fluorophenyl)- 3,4-dimethyl-2H-pyrazolo[3,4- |
| 37 | 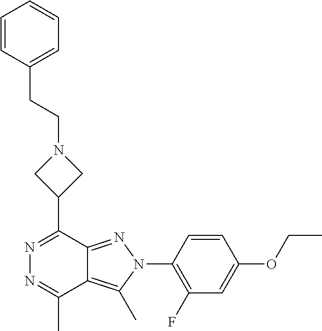 [SMILES: CCOc1ccc(-n2nc3c(C4CN(CCc5ccccc5)C4)nnc(C)c3c2C)c(F)c1] | 2-(4-Ethoxy-2-fluorophenyl)-3,4-dimethyl-7-(1- phenethylazetidin-3-yl)-2H-pyrazolo[3,4-d]pyridazine |
| 38 | 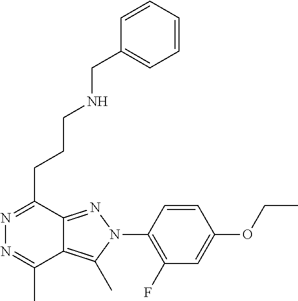 [SMILES: CCOc1ccc(-n2nc3c(CCCNCc4ccccc4)nnc(C)c3c2C)c(F)c1] | N-Benzyl-3-(2-(4-ethoxy-2-fluorophenyl)-3,4-dimethyl- 2H-pyrazolo[3,4-d]pyridazin-7-yl)propan-1-amine |
| 39 | 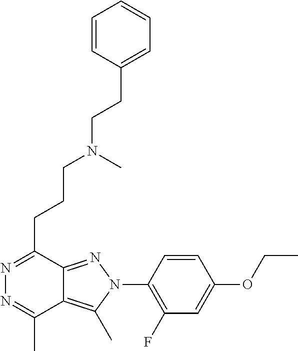 [SMILES: CCOc1ccc(-n2nc3c(CCCN(C)CCc4ccccc4)nnc(C)c3c2C)c(F)c1] | 3-(2-(4-Ethoxy-2-fluorophenyl)-3,4-dimethyl-2H- pyrazolo[3,4-d]pyridazin-7-yl)-N-methyl-N- phenethylpropan-1-amine |
| 40 | 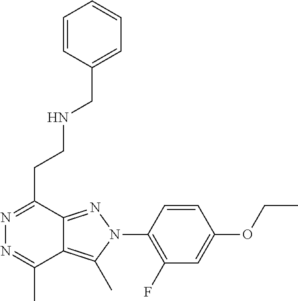 [SMILES: CCOc1ccc(-n2nc3c(CCNCc4ccccc4)nnc(C)c3c2C)c(F)c1] | N-Benzyl-2-(2-(4-ethoxy-2-fluorophenyl)-3,4-dimethyl- 2H-pyrazolo[3,4-d]pyridazin-7-yl)ethan-1-amine |
| 41 | 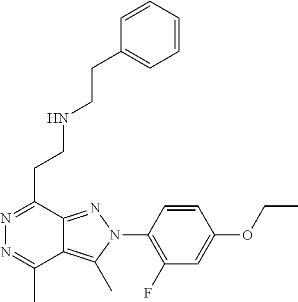 [SMILES: CCOc1ccc(-n2nc3c(CCNCCc4ccccc4)nnc(C)c3c2C)c(F)c1] | 2-(2-(4-Ethoxy-2-fluorophenyl)-3,4-dimethyl-2H- pyrazolo[3,4-d]pyridazin-7-yl)-N-phenethylethan-1-amine |
| 42 | 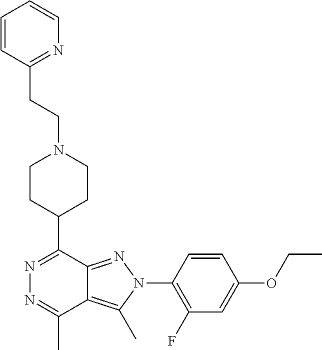 [SMILES: CCOc1ccc(-n2nc3c(C4CCN(CCc5ccccn5)CC4)nnc(C)c3c2C)c(F)c1] | 2-(4-Ethoxy-2-fluorophenyl)-3,4-dimethyl-7-(1-(2- (pyridin-2-yl)ethyl)piperidin-4-yl)-2H-pyrazolo[3,4- d]pyridazine |
| 43 | 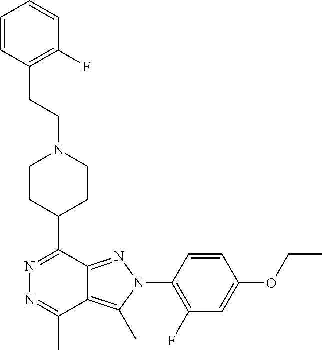 [SMILES: CCOc1ccc(-n2nc3c(C4CCN(CCc5ccccc5F)CC4)nnc(C)c3c2C)c(F)c1] | 2-(4-Ethoxy-2-fluorophenyl)-7-(1-(2- fluorophenethyl)piperidin-4-yl)-3,4-dimethyl-2H- pyrazolo[3,4-d]pyridazine |
| 44 | 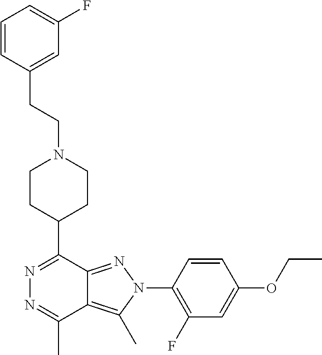 [SMILES: CCOc1ccc(-n2nc3c(C4CCN(CCc5cccc(F)c5)CC4)nnc(C)c3c2C)c(F)c1] | 2-(4-Ethoxy-2-fluorophenyl)-7-(1-(3- fluorophenethyl)piperidin-4-yl)-3,4-dimethyl-2H- pyrazolo[3,4-d]pyridazine |
| 45 | 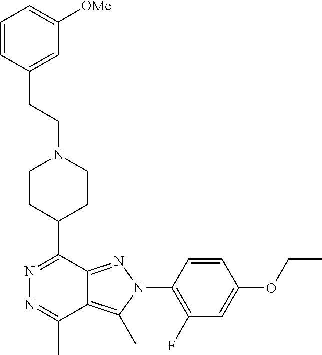 [SMILES: CCOc1ccc(-n2nc3c(C4CCN(CCc5cccc(OC)c5)CC4)nnc(C)c3c2C)c(F)c1] | 2-(4-Ethoxy-2-fluorophenyl)-7-(1-(3- methoxyphenethyl)piperidin-4-yl)-3,4-dimethyl-2H- pyrazolo[3,4-d]pyridazine |
| 46 | 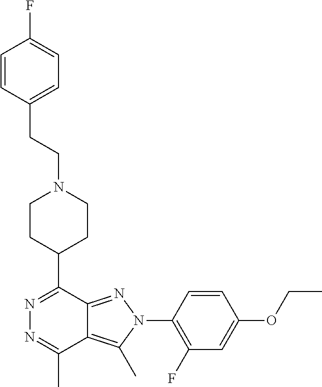 [SMILES: CCOc1ccc(-n2nc3c(C4CCN(CCc5ccc(F)cc5)CC4)nnc(C)c3c2C)c(F)c1] | 2-(4-Ethoxy-2-fluorophenyl)-7-(1-(4- fluorophenethyl)piperidin-4-yl)-3,4-dimethyl-2H- pyrazolo[3,4-d]pyridazine |
| 47 | 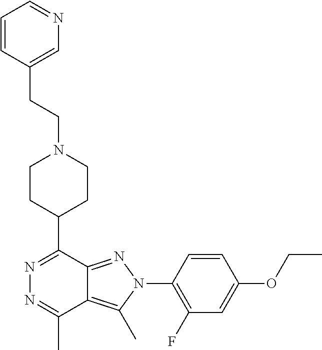 [SMILES: CCOc1ccc(-n2nc3c(C4CCN(CCc5cccnc5)CC4)nnc(C)c3c2C)c(F)c1] | 2-(4-Ethoxy-2-fluorophenyl)-3,4-dimethyl-7-(1-(2- (pyridin-3-yl)ethyl)piperidin-4-yl)-2H-pyrazolo[3,4- d]pyridazine |
| 48 | 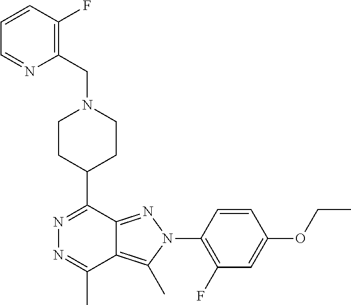 [SMILES: CCOc1ccc(-n2nc3c(C4CCN(Cc5ncccc5F)CC4)nnc(C)c3c2C)c(F)c1] | 2-(4-Ethoxy-2-fluorophenyl)-7-(1-((3-fluoropyridin-2- yl)methyl)piperidin-4-yl)-3,4-dimethyl-2H-pyrazolo[3,4- d]pyridazine |
| 49 | 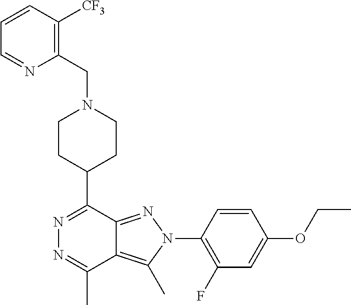 [SMILES: CCOc1ccc(-n2nc3c(C4CCN(Cc5ncccc5C(F)(F)F)CC4)nnc(C)c3c2C)c(F)c1] | 2-(4-Ethoxy-2-fluorophenyl)-3,4-dimethyl-7-(1-((3- (trifluoromethyl)pyridin-2-yl)methyl)piperidin-4-yl)-2H- pyrazolo[3,4-d]pyridazine |
| 50 | 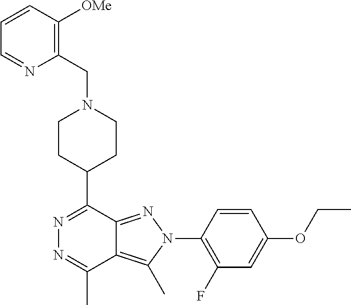 [SMILES: CCOc1ccc(-n2nc3c(C4CCN(Cc5ncccc5OC)CC4)nnc(C)c3c2C)c(F)c1] | 2-(4-Ethoxy-2-fluorophenyl)-7-(1-((3-methoxypyridin-2- yl)methyl)piperidin-4-yl)-3,4-dimethyl-2H-pyrazolo[3,4- d]pyridazine |
| 51 | 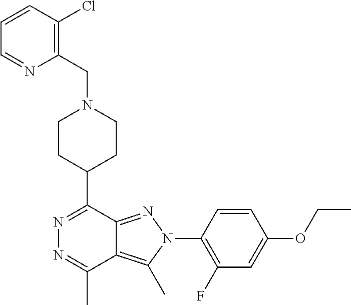 [SMILES: CCOc1ccc(-n2nc3c(C4CCN(Cc5ncccc5Cl)CC4)nnc(C)c3c2C)c(F)c1] | 7-(1-((3-Chloropyridin-2-yl)methyl)piperidin-4-yl)-2-(4- ethoxy-2-fluorophenyl)-3,4-dimethyl-2H-pyrazolo[3,4- d]pyridazine |
| 52 | 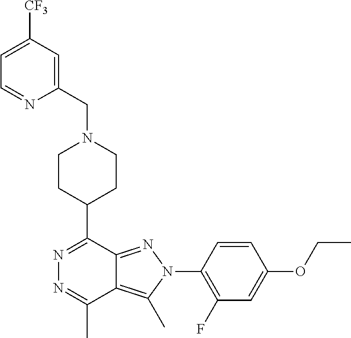 [SMILES: CCOc1ccc(-n2nc3c(C4CCN(Cc5cc(C(F)(F)F)ccn5)CC4)nnc(C)c3c2C)c(F)c1] | 2-(4-Ethoxy-2-fluorophenyl)-3,4-dimethyl-7-(1-((4- (trifluoromethyl)pyridin-2-yl)methyl)piperidin-4-yl)-2H- pyrazolo[3,4-d]pyridazine |
| 53 | 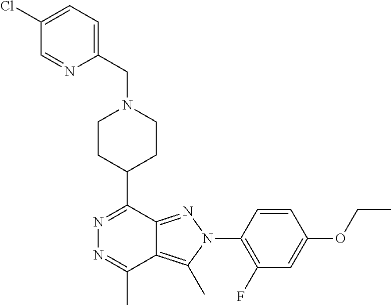 [SMILES: CCOc1ccc(-n2nc3c(C4CCN(Cc5ccc(Cl)cn5)CC4)nnc(C)c3c2C)c(F)c1] | 7-(1-((5-Chloropyridin-2-yl)methyl)piperidin-4-yl)-2-(4- ethoxy-2-fluorophenyl)-3,4-dimethyl-2H-pyrazolo[3,4- d]pyridazine |
| 54 | 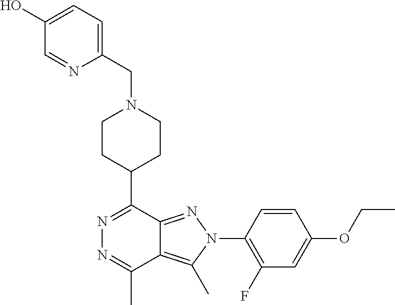 [SMILES: CCOc1ccc(-n2nc3c(C4CCN(Cc5ccc(O)cn5)CC4)nnc(C)c3c2C)c(F)c1] | 6-((4-(2-(4-Ethoxy-2-fluorophenyl)-3,4-dimethyl-2H- pyrazolo[3,4-d]pyridazin-7-yl)piperidin-1- yl)methyl)pyridin-3-ol |
| 55 | 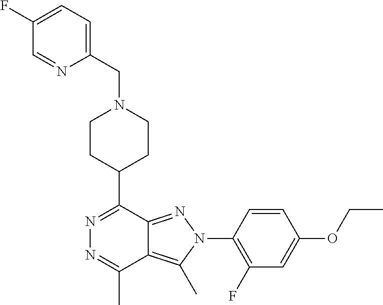 [SMILES: CCOc1ccc(-n2nc3c(C4CCN(Cc5ccc(F)cn5)CC4)nnc(C)c3c2C)c(F)c1] | 2-(4-Ethoxy-2-fluorophenyl)-7-(1-((5-fluoropyridin-2- yl)methyl)piperidin-4-yl)-3,4-dimethyl-2H-pyrazolo[3,4- d]pyridazine |
| 56 | 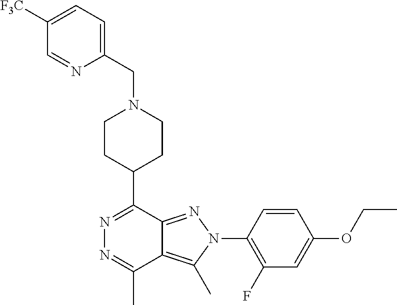 [SMILES: CCOc1ccc(-n2nc3c(C4CCN(Cc5ccc(C(F)(F)F)cn5)CC4)nnc(C)c3c2C)c(F)c1] | 2-(4-Ethoxy-2-fluorophenyl)-3,4-dimethyl-7-(1-((5- (trifluoromethyl)pyridin-2-yl)methyl)piperidin-4-yl)-2H- pyrazolo[3,4-d]pyridazine |
| 57 | 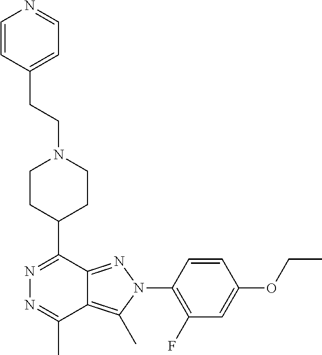 [SMILES: CCOc1ccc(-n2nc3c(C4CCN(CCc5ccncc5)CC4)nnc(C)c3c2C)c(F)c1] | 2-(4-Ethoxy-2-fluorophenyl)-3,4-dimethyl-7-(1-(2- (pyridin-4-yl)ethyl)piperidin-4-yl)-2H-pyrazolo[3,4- d]pyridazine |
| 58 | 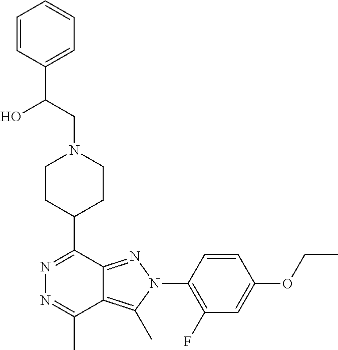 [SMILES: CCOc1ccc(-n2nc3c(C4CCN(CC(O)c5ccccc5)CC4)nnc(C)c3c2C)c(F)c1] | 2-(4-(2-(4-Ethoxy-2-fluorophenyl)-3,4-dimethyl-2H- pyrazolo[3,4-d]pyridazin-7-yl)piperidin-1-yl)-1- phenylethan-1-ol |
| 59 | 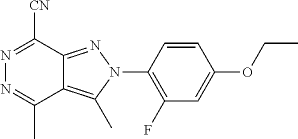 [SMILES: CCOc1ccc(-n2nc3c(C#N)nnc(C)c3c2C)c(F)c1] | 2-(4-Ethoxy-2-fluorophenyl)-3,4-dimethyl-2H- pyrazolo[3,4-d]pyridazine-7-carbonitrile |
| 60 | 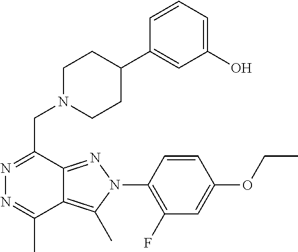 [SMILES: CCOc1ccc(-n2nc3c(CN4CCC(c5cccc(O)c5)CC4)nnc(C)c3c2C)c(F)c1] | 3-(1-((2-(4-Ethoxy-2-fluorophenyl)-3,4-dimethyl-2H- pyrazolo[3,4-d]pyridazin-7-yl)methyl)piperidin-4- yl)phenol |
| 61 | 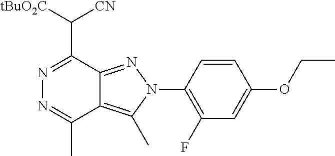 [SMILES: CCOc1ccc(-n2nc3c(C(C#N)C(=O)OC(C)(C)C)nnc(C)c3c2C)c(F)c1] | tert-Butyl 2-cyano-2-(2-(4-ethoxy-2-fluorophenyl)-3,4- dimethyl-2H-pyrazolo[3,4-d]pyridazin-7-yl)acetate |
| 62 | 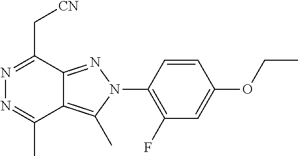 [SMILES: CCOc1ccc(-n2nc3c(CC#N)nnc(C)c3c2C)c(F)c1] | 2-(2-(4-Ethoxy-2-fluorophenyl)-3,4-dimethyl-2H- pyrazolo[3,4-d]pyridazin-7-yl)acetonitrile |
| 63 | 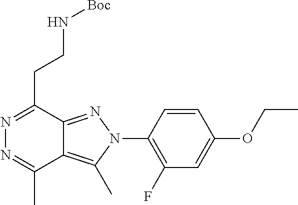 [SMILES: CCOc1ccc(-n2nc3c(CCNC(=O)OC(C)(C)C)nnc(C)c3c2C)c(F)c1] | tert-Butyl (2-(2-(4-ethoxy-2-fluorophenyl)-3,4-dimethyl- 2H-pyrazolo[3,4-d]pyridazin-7-yl)ethyl)carbamate |
| 64 | 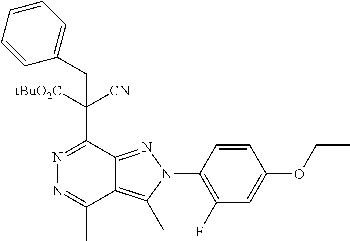 [SMILES: CCOc1ccc(-n2nc3c(C(C#N)(Cc4ccccc4)C(=O)OC(C)(C)C)nnc(C)c3c2C)c(F)c1] | tert-Butyl 2-cyano-2-(2-(4-ethoxy-2-fluorophenyl)-3,4- dimethyl-2H-pyrazolo[3,4-d]pyridazin-7-yl)-3- phenylpropanoate |
| 65 | 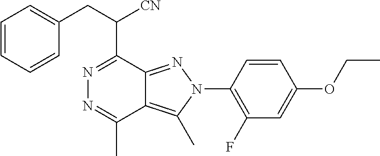 [SMILES: CCOc1ccc(-n2nc3c(C(C#N)Cc4ccccc4)nnc(C)c3c2C)c(F)c1] | 2-(2-(4-Ethoxy-2-fluorophenyl)-3,4-dimethyl-2H- pyrazolo[3,4-d]pyridazin-7-yl)-3-phenylpropanenitrile |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

R₁ is selected from substituted or unsubstituted C₁₋₆ alkyl, substituted or unsubstituted C₂₋₆ alkenyl and substituted or unsubstituted C₂₋₆ alkynyl;
wherein the alkyl, alkenyl or alkynyl in R₁, if substituted, is substituted with one or more substituent/s selected from —OR₁₁, —C(O)R₁₁, halogen, —CN, haloalkyl, haloalkoxy and —NR₁₁R_{11′″};
wherein R₁₁ is selected from hydrogen, unsubstituted C₁₋₆ alkyl, unsubstituted C₂₋₆ alkenyl and unsubstituted C₂₋₆ alkynyl;
and R_{11′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

R₁ is substituted or unsubstituted C₁₋₆ alkyl;
wherein the alkyl, alkenyl or alkynyl in R₁, if substituted, is substituted with one or more substituent/s selected from —OR₁₁, —C(O)R₁₁, halogen, —CN, haloalkyl, haloalkoxy and —NR₁₁R_{11′″};
wherein R₁₁ is selected from hydrogen, unsubstituted C₁₋₆ alkyl, unsubstituted C₂₋₆ alkenyl and unsubstituted C₂₋₆ alkynyl;
and R_{11′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

R₂ is selected from —NR₇R_{7′″}, —CN and substituted or unsubstituted N-containing-heterocyclyl;
wherein said N-containing-heterocyclyl in R₂, if substituted, is mono-substituted with R₇ₐ;
wherein R₇ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc;
and wherein R_{7′″} is selected from hydrogen, substituted or unsubstituted C₁₋₆ alkyl, substituted or unsubstituted C₂₋₆ alkenyl and substituted or unsubstituted C₂₋₆ alkynyl;
wherein R₇ₐ is selected from substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl and -Boc;
wherein, the alkyl, alkenyl or alkynyl, also in alkylaryl, alkyheterocyclyl or alkycycloalkyl, in R₇ₐ, if substituted, is substituted with one or more substituent/s selected from —OR₁₂, halogen, —CN, haloalkyl, haloalkoxy and —NR₁₂R_{12′″};
and wherein, the aryl, heterocyclyl or cycloalkyl, also in alkylaryl, alkyheterocyclyl or alkycycloalkyl, in R₇ₐ, if substituted, is substituted with one or more substituent/s selected from halogen, —R₁₂, —OR₁₂, —NO₂, —NR₁₂R_{12′″}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, S(O)R₁₂, S(O)₂R₁₂, —CN, haloalkyl, haloalkoxy, —C(O)OR₁₂, —C(O)NR₁₂R_{12′}, —NR₁₂S(O)₂NR_{12′}R_{12″}, unsubstituted alkylcycloalkyl and unsubstituted alkylaryl, unsubstituted alkylheterocyclyl;
wherein R₁₂, R_{12′} and R₁₂″ are independently selected from hydrogen, unsubstituted C₁₋₆ alkyl, unsubstituted C₂₋₆ alkenyl and unsubstituted C₂₋₆ alkynyl;
and wherein R_{12′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

wherein, the alkyl, alkenyl or alkynyl, also in alkylaryl, alkyheterocyclyl or alkycycloalkyl, other than those defined in R₁, R₂ or R₇ₐ, if substituted, is substituted with one or more substituent/s selected from —OR₁₃, halogen, —CN, haloalkyl, haloalkoxy and —NR₁₃R_{13′″};
wherein R₁₃ is selected from hydrogen, unsubstituted C₁₋₆ alkyl, unsubstituted C₂₋₆ alkenyl, and unsubstituted C₂₋₆ alkynyl;
and wherein R_{13′″} is selected from hydrogen, unsubstituted C₁₋₈ alkyl, unsubstituted C₂₋₈ alkenyl, unsubstituted C₂₋₈ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the invention the compound of general Formula (I),

the aryl, heterocyclyl or cycloalkyl, also in alkylaryl, alkyheterocyclyl or alkycycloalkyl, other than those defined in R₁, R₂ or R₇ₐ, if substituted, is substituted with one or more substituent/s selected from halogen, —R₁₄, —OR₁₄, —NO₂, —NR₁₄R_{14′″}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄ , —S(O)R₁₄, S(O)₂R₁₄, —CN, haloalkyl, haloalkoxy, —C(O)OR₁₄, —C(O)NR₁₄R_{14′}, —NR₁₄S(O)₂NR_{14′}R_{14′} and C(CH₃)₂—OR₁₄;
wherein R₁₄, R_{14′} and R_{14″} are independently selected from hydrogen, unsubstituted C₁₋₆ alkyl, unsubstituted C₂₋₆ alkenyl, unsubstituted C₂₋₆ alkynyl, unsubstituted aryl, unsubstituted cycloalkyl and unsubstituted heterocyclyl;
and wherein R_{14′″} is selected from hydrogen, unsubstituted C₁₋₆ alkyl, unsubstituted C₂₋₆ alkenyl, unsubstituted C₂₋₆ alkynyl and -Boc;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the alkyl, alkenyl or alkynyl in R₁, if substituted, is substituted with one or more substituent/s selected from —OR₁₁, —C(O)R₁₁, halogen, —CN, haloalkyl, haloalkoxy and —NR₁₁R_{11′″};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the N-containing-heterocyclyl in R₂, if substituted, is mono-substituted with R₇ₐ;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the N-containing-heterocyclyl in R₂, if substituted, is substituted with R₇ₐ;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),
wherein, the alkyl, alkenyl or alkynyl, also in alkylaryl, alkyheterocyclyl or alkycycloalkyl, in R₇ₐ, if substituted, is substituted with one or more substituent/s selected from —OR₁₂, halogen, —CN, haloalkyl, haloalkoxy and —NR₁₂R_{12′″};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the aryl, heterocyclyl or cycloalkyl, also in alkylaryl, alkyheterocyclyl or alkycycloalkyl, in R₇ₐ, if substituted, is substituted with one or more substituent/s selected from halogen, —R₁₂, —OR₁₂, —NO₂, —NR₁₂R_{12′″}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂ , —S(O)R₁₂, S(O)₂R₁₂, —CN, haloalkyl, haloalkoxy, —C(O)OR₁₂, —C(O)NR₁₂R_{12′}, —NR₁₂S(O)₂NR_{12′}R_{12″}, unsubstituted alkylcycloalkyl, unsubstituted alkylaryl, and unsubstituted alkylheterocyclyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

wherein, the alkyl, alkenyl or alkynyl, also in alkylaryl, alkyheterocyclyl or alkycycloalkyl, other than those defined in R₁, R₂ or R₇ₐ, if substituted, is substituted with one or more substituent/s selected from —OR₁₃, halogen, —CN, haloalkyl, haloalkoxy and —NR₁₃R_{13′″};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of general Formula (I),

the aryl, heterocyclyl or cycloalkyl, also in alkylaryl, alkyheterocyclyl or alkycycloalkyl, other than those defined in R₁, R₂ or R₇ₐ, if substituted, is substituted with one or more substituent/s selected from halogen, —R₁₄, —OR₁₄, —NO₂, —NR₁₄R_{14′″}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄ , —S(O)R₁₄, S(O)₂R₁₄, —CN, haloalkyl, haloalkoxy, —C(O)OR₁₄, —C(O)NR₁₄R_{14′}, —NR₁₄S(O)₂NR_{14′}R_{14″} and C(CH₃)₂—OR₁₄;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of general Formula (I),

the halogen is fluorine, chlorine, iodine or bromine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a most preferred embodiment of the compound according to the invention of general Formula (I)

the halogen is fluorine;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of general Formula (I),

the haloalkyl is —CF3 ;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment of the compound according to the invention of general Formula (I),

the haloalkoxy is —OCF3;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

As this invention is aimed at providing a compound or a chemically related series of compounds which act as dual ligands of the α2δ subunit, particularly the α2δ-1 subunit, of the voltage-gated calcium channel and the μ-opioid receptor it is a very preferred embodiment in which the compounds are selected which act as dual ligands of the α2δ subunit, particularly the α2δ-1 ubunit, of the voltage-gated calcium channel and the μ-opioid receptor and especially compounds which have a binding expressed as Kᵢ responding to the following scales:

Kᵢ(μ) is preferably<1000 nM, more preferably<500 nM, even more preferably<100 nM.

Kᵢ(α2δ1) is preferably<10000 nM, more preferably<5000 nM, even more preferably<500 nM or even more preferably<100 nM.

In the following the phrase “compound of the invention” is used. This is to be understood as any compound according to the invention as described above according to general Formula (In), (I), (I′), (I²′), (I³′), (I⁴′), (I⁵′), (I⁶′), (I^{7a}′) or (I^{7b}′) or (I⁸′).

The compounds of the invention represented by the above described Formula (I) may include enantiomers depending on the presence of chiral centres or isomers depending on the presence of multiple bonds (e.g. Z, E). The single isomers, enantiomers or diastereoisomers and mixtures thereof fall within the scope of the present invention.

For the sake of clarity the expression “a compound according to Formula (I), wherein e.g. R₁, R₂, R₃, R₄, R₅, R_{5′}, R₆, R_{6′}, X and m are as defined in the description” would (just like the expression “a compound of Formula (I) as defined in any one of claims e.g. 1 to 8” found in the claims) refer to “a compound according to Formula (I)”, wherein the definitions of the respective substituents R₁ etc. (also from the cited claims) are applied. In addition, this would also mean, though (especially in regards to the claims) that also one or more disclaimers defined in the description (or used in any of the cited claims like e.g. claim 1) would be applicable to define the respective compound. Thus, a disclaimer found in e.g. claim 1 would be also used to define the compound “of Formula (I) as defined in any one of claims e.g. 1 to 8”.

For the sake of clarity the expression “a compound according to Formula (In), wherein R₁, R₂, R₃, R₄, R₅, R_{5′}, R₆, R_{6′}, X and m and n are as defined in the description” would (just like the expression “a compound of Formula (In) as defined in any one of claims 1 to 8” found in the claims) refer to “a compound according to Formula (In)”, wherein the definitions of the respective substituents R₁ etc. (also from the cited claims) are applied. In addition, this would also mean, though (especially in regards to the claims) that also one or more disclaimers defined in the description (or used in any of the cited claims like e.g. claim 1) would be applicable to define the respective compound. Thus, a disclaimer found in e.g. claim 1 would be also used to define the compound “of Formula (In) as defined in any one of claims 1 to 8”.

In general the processes are described below in the experimental part. The starting materials are commercially available or can be prepared by conventional methods.

A preferred aspect of the invention is also a process for the production of a compound according to Formula (I), following scheme 1.

A preferred embodiment of the invention is a process for the production of a compound according to Formula (I), wherein, if not defined otherwise, m, R₁, R₂, R₃, R₄, R₅, R_{5′}, R₆, R_{6′}, Rₓ, R_{x′} and X have the meanings defined in the description.

In a particular embodiment there is a process for the production of a compound according to Formula (I), wherein the compound of Formula (I) is a compound of Formula (Ia), wherein X is —CH═CH—

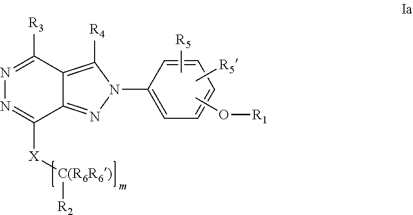

said process comprises the reaction of a compound of general formula II, wherein Y is an halogen, preferably chlorine,

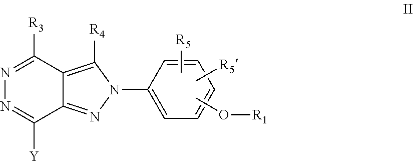

with a boronic acid (Z═H) or boronic ester (Z=Alkyl) of general formula III

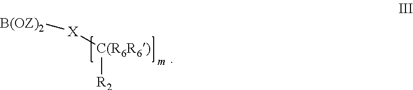

In a particular embodiment there is a process for the production of a compound according to Formula (I), wherein the compound of Formula (I) is a compound of Formula (Ib), wherein X is —CH₂CH₂—,

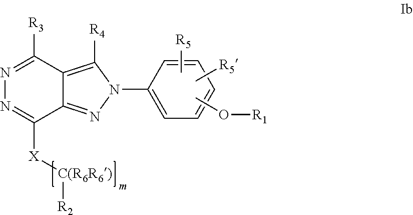

said process comprises the reduction of compounds of formula Ia, wherein X is —CH═CH—

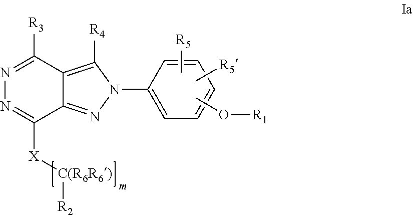

using suitable reductive reagents, preferably hydrogen in the presence of a catalyst, preferably Pd(OH)₂ on carbon, in an organic solvent, preferably MeOH.

In a particular embodiment there is a process for the production of a compound according to Formula (I), wherein the compound of Formula (I) is a compound of Formula (Ib), wherein X is —CH₂CH₂—

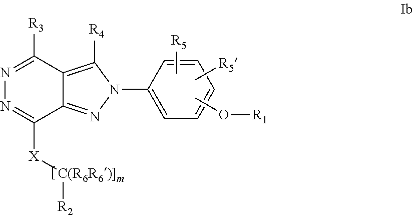

said process comprises the reaction of a compound of formula II with an organometalic reagent, prepared from a compound of general formula IV,

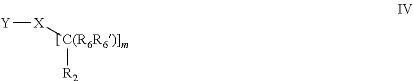

with a metal agent, preferably Zn.

In a particular embodiment there is a process for the production of a compound according to Formula (I), wherein the compound of Formula (I) is a compound of Formula (Ic),

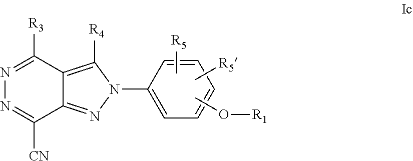

said process comprises the reaction of a compound of formula II,

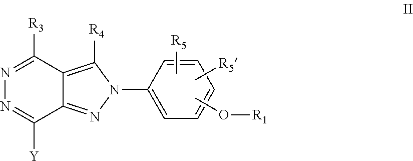

wherein Y is an halogen, preferably chlorine, with a cyanation reagent, preferably zinc cyanide, in the presence of a Pd catalyst.

In a particular embodiment there is a process for the production of a compound according to Formula (I), wherein the compound of Formula (I) is a compound of Formula (Id),

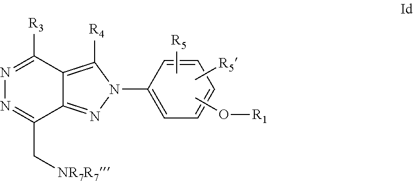

said process comprises treating a compound of formula Ic

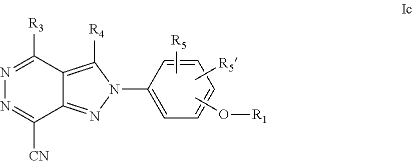

with an acid, preferably HCl, followed by a reduction reaction and final reductive amination with an amine of formula V,

NHR₇R_{7′″} V.

In a particular embodiment there is a process for the production of a compound according to Formula (I), wherein the compound of Formula (I) is a compound of Formula (Ie),

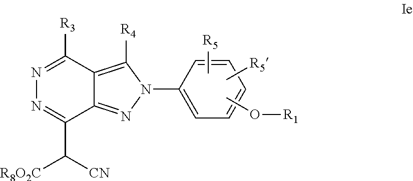

said process comprises the reaction of a compound of formula II,

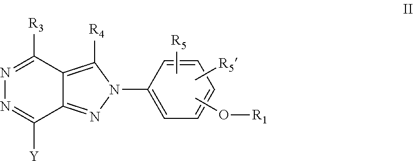

wherein Y is an halogen, preferably chlorine, with a reagent of formula VI,

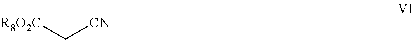

in the presence of a base.

In a particular embodiment there is a process for the production of a compound according to Formula (I), wherein the compound of Formula (I) is a compound of Formula (If),

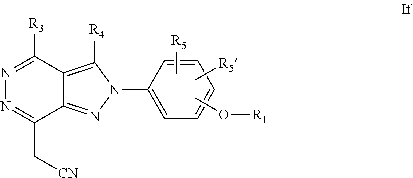

said process comprises the transformation of a compound of formula Ie,

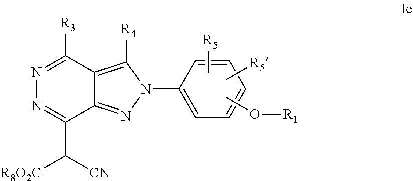

by heating at a suitable temperature, such as in the range of 50-180° C., in an organic solvent.

In a particular embodiment there is a process for the production of a compound according to Formula (I), wherein the compound of Formula (I) is a compound of Formula (Ig),

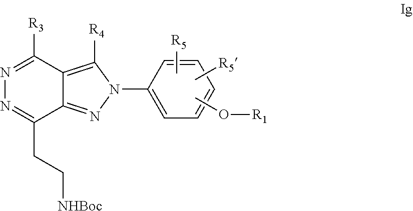

said process comprises the reduction of a compound of formula If

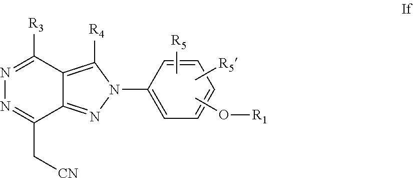

with a suitable reductive reagent, preferably sodium borohydride in the presence of NiCl₂.6H₂O and ditert-butyl dicarbonate, in an organic solvent.

In a particular embodiment there is a process for the production of a compound according to Formula (I), wherein the compound of Formula (I) is a compound of Formula (Ih),

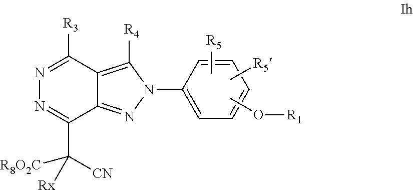

said process comprises the alkylation reaction of a compound of formula Ie

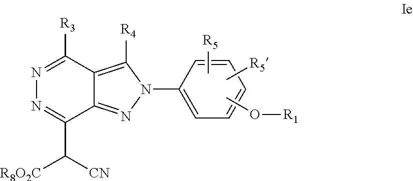

with a reagent of general formula VII

RxY VII

wherein Y is a good leaving group such as an halogen or sulfonate, in the presence of a base, preferably NaH, in an organic solvent.

In a particular embodiment there is a process for the production of a compound according to Formula (I), wherein the compound of Formula (I) is a compound of Formula (Ii),

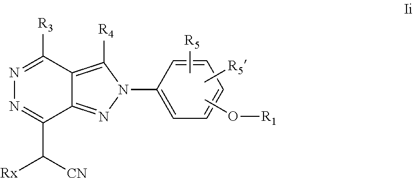

said process comprises the transformation of a compound of formula Ih,

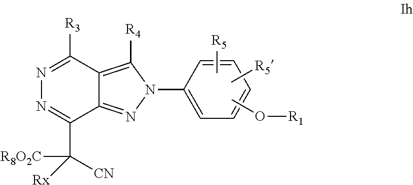

by heating at a suitable temperature, such as in the range of 50-180° C., in an organic solvent, preferably hexafluoro-2-isopropanol, alternatively under microwave irradiation.

In a particular embodiment there is a process for the production of a compound according to Formula (I), by the reduction reaction of a carbonyl derivative with a suitable reductive reagent, preferably sodium borohydride, in an organic solvent, preferably MeOH, to afford a hydroxyl compound.

In a particular embodiment there is a process for the production of a compound according to Formula (I), by deprotection reaction of a compound of formula I that contains an amine protecting group such as a carbamate, preferably tert-butoxy carbonyl, by any suitable method, such as treatment with an acid, preferably HCl or trifluoroacetic acid in an appropriate solvent such as 1,4-dioxane, DCM, ethyl acetate or a mixture of an organic solvent and water.

In a particular embodiment there is a process for the production of a compound according to Formula (I), by reductive amination reaction of a compound of formula I that contains an amino group with an aldehyde, preferably carried out with a reductive reagent, preferably sodium triacetoxyborohydride, in an organic solvent, preferably DCE, in the presence of an organic base, preferably DIPEA or TEA. Alternatively, the reaction can be carried out in the presence of an acid, preferably acetic acid.

In a particular embodiment there is a process for the production of a compound according to Formula (I), by reaction of a compound of formula I that contains an amino group with an alkylating reagent, in the presence of a base, preferably DIPEA or K₂CO₃, in an organic solvent, preferably acetonitrile, at suitable temperature, such as in the range of 0-120° C.

In a particular embodiment there is a process for the production of a compound according to Formula (I), by reaction of a compound of formula I that contains an amino group with a vinyl derivative, in an organic solvent, preferably 2-methoxyethanol, at suitable temperature, such as in the range of 20-140° C.

In a particular embodiment a compound of Formula (II),

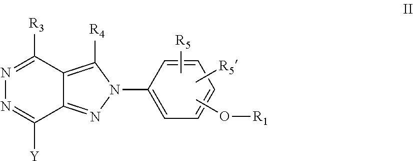

is used for the preparation of compounds of Formula (I).

In a particular embodiment a compound of Formula (III),

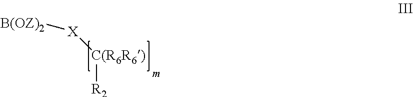

is used for the preparation of compounds of Formula (I).

In a particular embodiment a compound of Formula (IV),

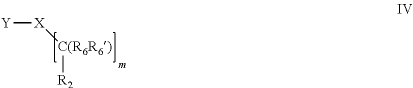

is used for the preparation of compounds of Formula (I).

In a particular embodiment a compound of Formula (V),

NHIR₇R_{7′″} V

is used for the preparation of compounds of Formula (I).

In a particular embodiment a compound of Formula (VI),

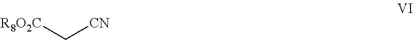

is used for the preparation of compounds of Formula (I).

In a particular embodiment a compound of Formula (VII),

RxY VII

is used for the preparation of compounds of Formula (I).

In a particular embodiment a compound of Formula (Ia), wherein X is —CH═CH—

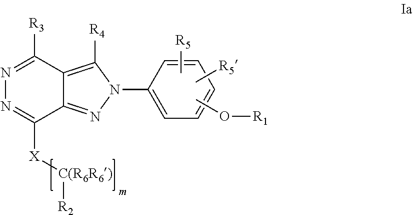

is used for the preparation of compounds of Formula (I).

In a particular embodiment a compound of Formula (Ib), wherein X is ——CH₂CH₂—

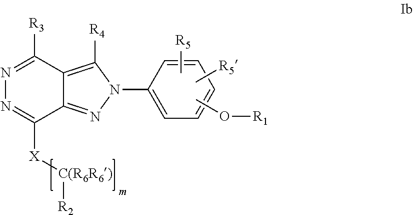

is used for the preparation of compounds of Formula (I).

In a particular embodiment a compound of Formula (Ic),

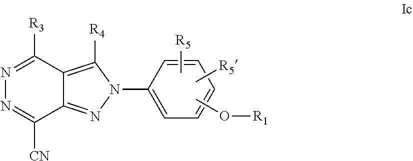

is used for the preparation of compounds of Formula (I).

In a particular embodiment a compound of Formula (Id),

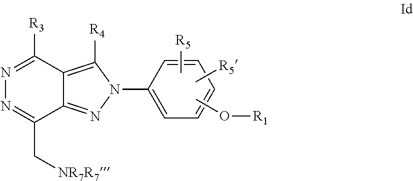

is used for the preparation of compounds of Formula (I).

In a particular embodiment a compound of Formula (Ie),

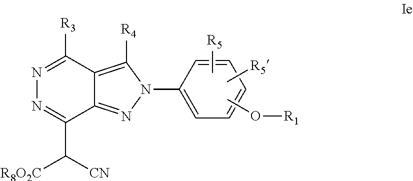

is used for the preparation of compounds of Formula (I).

In a particular embodiment a compound of Formula (If),

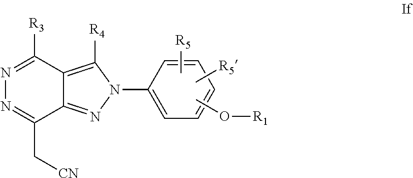

is used for the preparation of compounds of Formula (I).

In a particular embodiment a compound of Formula (Ig),

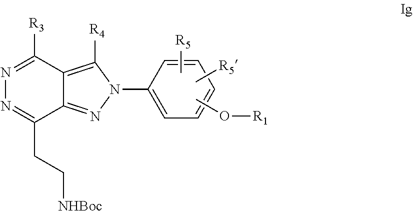

is used for the preparation of compounds of Formula (I).

In a particular embodiment a compound of Formula (Ih),

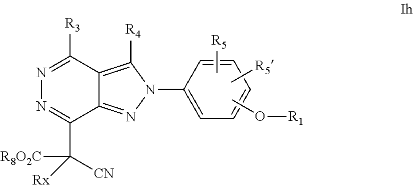

is used for the preparation of compounds of Formula (I).

In a particular embodiment a compound of Formula (Ih),

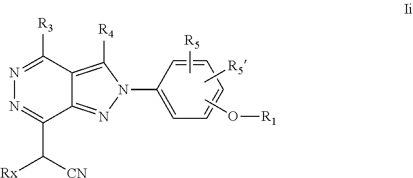

is used for the preparation of compounds of Formula (I).

The obtained reaction products may, if desired, be purified by conventional methods, such as crystallisation and chromatography. Where the above described processes for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

One preferred pharmaceutically acceptable form of a compound of the invention is the crystalline form, including such form in pharmaceutical composition. In the case of salts and also solvates of the compounds of the invention the additional ionic and solvent moieties must also be non-toxic. The compounds of the invention may present different polymorphic forms, it is intended that the invention encompasses all such forms.

Another aspect of the invention refers to a pharmaceutical composition which comprises a compound according to the invention as described above according to general formula I or a pharmaceutically acceptable salt or steroisomer thereof, and a pharmaceutically acceptable carrier, adjuvant or vehicle. The present invention thus provides pharmaceutical compositions comprising a compound of this invention, or a pharmaceutically acceptable salt or stereoisomers thereof together with a pharmaceutically acceptable carrier, adjuvant, or vehicle, for administration to a patient.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the apropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition, or be provided as a separate composition for administration at the same time or at different time.

Another aspect of the invention refers to the use of a compound of the invention or a pharmaceutically acceptable salt or isomer thereof in the manufacture of a medicament.

Another aspect of the invention refers to a compound of the invention according as described above according to general formula I, or a pharmaceutically acceptable salt or isomer thereof, for use as a medicament for the treatment of pain. Preferably the pain is medium to severe pain, visceral pain, chronic pain, cancer pain, migraine, inflammatory pain, acute pain or neuropathic pain, allodynia or hyperalgesia. This may include mechanical allodynia or thermal hyperalgesia.

Another aspect of the invention refers to the use of a compound of the invention in the manufacture of a medicament for the treatment or prophylaxis of pain.

In a preferred embodiment the pain is selected from medium to severe pain, visceral pain, chronic pain, cancer pain, migraine, inflammatory pain, acute pain or neuropathic pain, allodynia or hyperalgesia, also preferably including mechanical allodynia or thermal hyperalgesia.

Another aspect of this invention relates to a method of treating or preventing pain which method comprises administering to a patient in need of such a treatment a therapeutically effective amount of a compound as above defined or a pharmaceutical composition thereof. Among the pain syndromes that can be treated are medium to severe pain, visceral pain, chronic pain, cancer pain, migraine, inflammatory pain, acute pain or neuropathic pain, allodynia or hyperalgesia, whereas this could also include mechanical allodynia or thermal hyperalgesia.

The present invention is illustrated below with the aid of examples. These illustrations are given solely by way of example and do not limit the general spirit of the present invention.

General Experimental Part (Methods and Equipment of the Synthesis and Analysis

Method Description

A process is described in Scheme 1 for the preparation of compounds of general formula I, wherein R₁ to R_{6′}, Rₓ, R_{x′}, m and X have the meanings defined above.

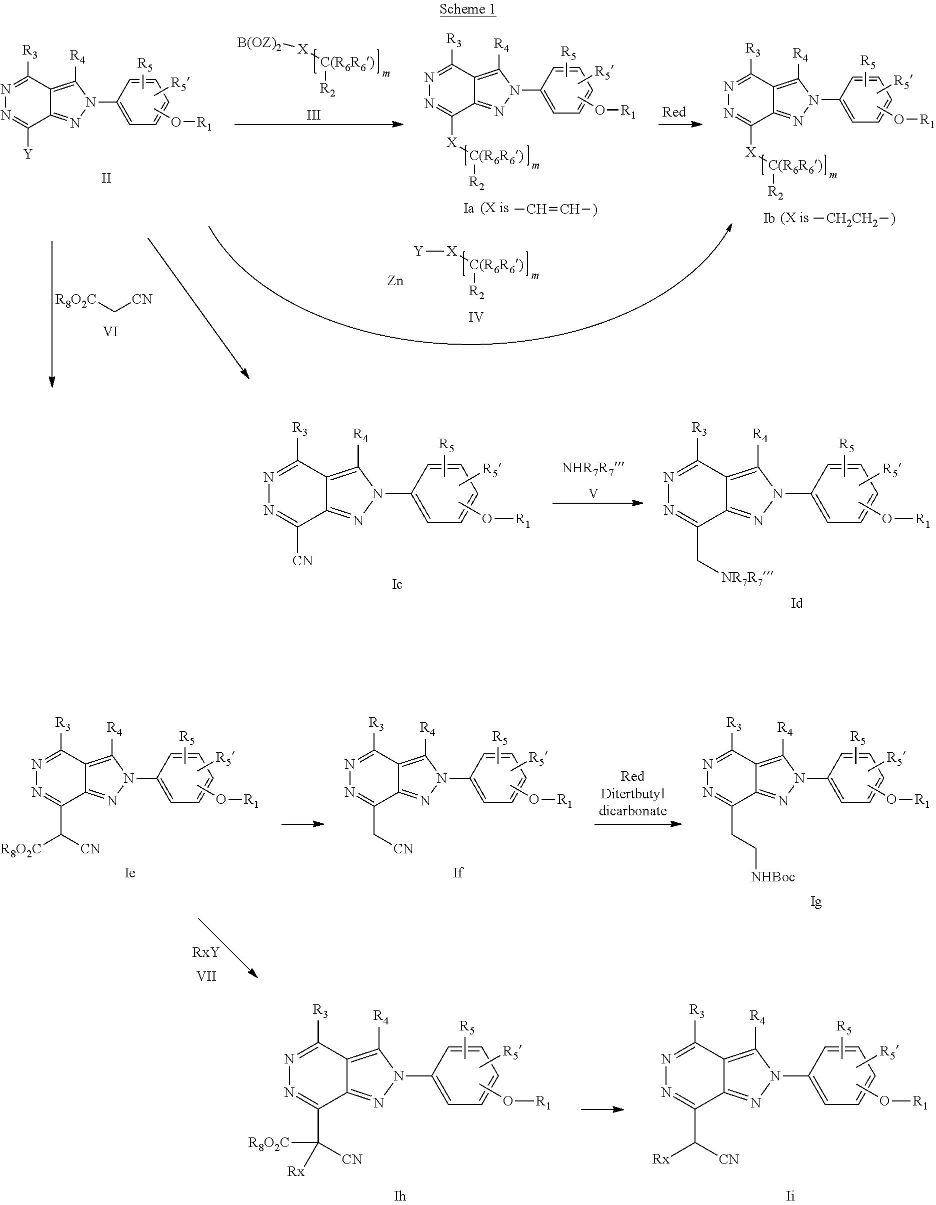

Compounds of general formula Ia, where the group defined by X—[C(R₆R′₆)]ₘ—R₂ is attached to the central pyridazine core by an unsaturated carbon atom (X is —CH═CH—), are prepared by reaction of a compound of general formula II, wherein Y is an halogen, preferably chlorine, with a boronic acid (Z═H) or boronic ester (Z=Alkyl) of general formula III and is carried out in the presence of a catalyst, preferably tetrakis(triphenylphosphine)palladium, a base, preferably Na₂CO₃, in an organic solvent, preferably 1,4-dioxane, or a mixture of organic solvents and water, preferably toluene/EtOH/water, at a suitable temperature, preferably in the range of 25-130° C., alternatively under microwave irradiation.

Compounds of formula Ib, where the group defined by X—[C(R₆R′₆)]ₘ—R₂ is attached to the central pyridazine core by a saturated carbon atom (X is —CH₂CH₂—), can be prepared by reducing compounds of formula Ia using suitable reductive reagents, preferably hydrogen in the presence of a catalyst, preferably Pd(OH)₂ on carbon, in an organic solvent, preferably MeOH.

Alternatively, compounds of formula Ib can be prepared by reaction of a compound of formula II with an organometalic reagent, prepared from a compound of general formula IV, with a metal agent, preferably Zn and in the presence of a catalyst, preferably tetrakis(triphenylphosphine)palladium, in an organic solvent, preferably DMF, at a temperature range of 50-180° C., alternatively under microwave irradiation.

Compounds of general formula Ic can be prepared by the reaction of a compound of formula II, wherein Y is an halogen, preferably chlorine, with a cyanation reagent, preferably zinc cyanide, in the presence of a Pd catalyst, preferably tetrakis(triphenylphosphine)palladium, in an organic solvent, preferably DMF, at a suitable temperature, such in the range of 20-180° C., alternatively under microwave irradiation.

Compounds of general formula Id can be prepared by the reaction of a compound of formula Ic by treating with an acid, preferably HCl, in the presence of an alcohol, preferably ethanol, at suitable temperature, such as in the range of 0-130° C., followed by reduction with a suitable reductive reagent, preferably diisobutylaluminium hydride, in an organic solvent, preferably DCE and final reductive amination with an amine of formula V, in the presence of a reductive reagent, preferably sodium triacetoxyborohydride, in an organic solvent, preferably DCE, alternatively in the presence of an organic base, preferably DIPEA or TEA.

Compounds of general formula Ie can be prepared by the reaction of a compound of formula II, wherein Y is an halogen, preferably chlorine, with a reagent of formula VI, in the presence of a base, preferably Cs₂CO₃, in an organic solvent, preferably DMSO, at suitable temperature, such as in the range of 20-180° C., alternatively under microwave irradiation,

Compounds of formula If can be prepared by the transformation of a compound of formula Ie, by heating at a suitable temperature, such as in the range of 50-180° C., in an organic solvent, preferably hexafluoro-2-isopropanol, alternatively under microwave irradiation.

Compounds of formula Ig can be prepared by the reduction of a compound of formula If with a suitable reductive reagent, preferably sodium borohydride in the presence of NiCl₂.6H₂O and ditert-butyl dicarbonate, in an organic solvent, preferably MeOH.

Compounds of formula Ih can be prepared by the alkylation reaction of a compound of formula Ie with a reagent of general formula VII wherein Y is a good leaving group such as an halogen or sulfonate, in the presence of a base, preferably NaH, in an organic solvent, preferably DMF, at suitable temperature, such as in the range of 0-80° C.

Compounds of formula Ii can be prepared by the transformation of a compound of formula Ih, by heating at a suitable temperature, such as in the range of 50-180° C., in an organic solvent, preferably hexafluoro-2-isopropanol, alternatively under microwave irradiation.

Additionally, different interconversion methods can be used to prepare compounds of general formula I:

By the reduction reaction of a carbonyl derivative with a suitable reductive reagent, preferably sodium borohydride, in an organic solvent, preferably MeOH, to afford a hydroxyl compound.

By deprotection reaction of a compound of formula I that contains an amine protecting group such as a carbamate, preferably tert-butoxy carbonyl, by any suitable method, such as treatment with an acid, preferably HCl or trifluoroacetic acid in an appropriate solvent such as 1,4-dioxane, DCM, ethyl acetate or a mixture of an organic solvent and water.

By reductive amination reaction of a compound of formula I that contains an amino group with an aldehyde, preferably carried out with a reductive reagent, preferably sodium triacetoxyborohydride, in an organic solvent, preferably DCE, in the presence of an organic base, preferably DIPEA or TEA.

Alternatively, the reaction can be carried out in the presence of an acid, preferably acetic acid.

By reaction of a compound of formula I that contains an amino group with an alkylating reagent, in the presence of a base, preferably DIPEA or K₂CO₃, in an organic solvent, preferably acetonitrile, at suitable temperature, such as in the range of 0-120° C.

By reaction of a compound of formula I that contains an amino group with a vinyl derivative, in an organic solvent, preferably 2-methoxyethanol, at suitable temperature, such as in the range of 20-140° C.

Compounds II, III, IV, V, VI and VII are commercially available or can be prepared from commercially available reagents using methods described in the literature.

### EXAMPLES

### Intermediates and Examples

The following abbreviations are used in the examples:

ACN: Acetonitrile

Anh: Anhydrous

Aq: Aqueous

Conc: Concentrated

CH: Cyclohexane

DCM: Dichloromethane

DCE: 1,2-Dichloroethane

DIPEA: N,N-Diisopropylethylamine

DMAP: N,N-dimethylpyridin-4-amine

DMSO: Dimethylsulfoxide

EtOAc: Ethyl acetate

EtOH: Ethanol

Ex: Example

h: Hour/s

HPLC: High-performance liquid chromatography

HRMS: High-resolution mass spectrometry

INT: Intermediate

MeOH: Methanol

MS: Mass spectrometry

Min: Minutes

Pd(PPh₃)₄: tetrakis(triphenylphosphine)palladium(0)

Quant: Quantitative

Ret: Retention

rt: Room temperature

Sat: Saturated

TEA: Et₃N, Triethylamine

TFA: Trifluoroacetic acid

THF: Tetrahydrofuran

Wt: Weight

The following methods were used to generate the HPLC or HPLC-MS data:

Method A: Column Eclipse XDB-C18 4.6×150 mm, 5 μm; flow rate 1 mL/min; A: H₂O (0.05% TFA); B: ACN; gradient: 5% to 95% B in 7 min, isocratic 95% B 5 min.

Method B: Column Zorbax SB-C18 2.1×50 mm, 1.8 μm; flow rate 0.5 mL/min; A: H₂O (0.1% formic acid); B: ACN (0.1% formic acid); gradient: 5% to 95% B in 4 min, isocratic 95% B 4 min.

INT 1. 7-Chloro-2-(4-ethoxy-2-fluorophenyl)-3,4-dimethyl-2H-pyrazolo[3,4-d]pyridazine.
a) (Z)-Ethyl 2-chloro-2-(2-(4-ethoxy-2-fluorophenyl)hydrazono)acetate: To a solution of 4-ethoxy-2-fluoroaniline (36.9 g, 237.8 mmol) in a mixture of conc HCl:EtOH (1:1, 118 mL) cooled at 0° C., a solution of NaNO₂ (17.88 g, 259 mmol) in water (89 mL) was added dropwise. After stirring 20 min at 0° C., ethyl 2-chloro-3-oxobutanoate (32.89 mL, 273 mmol) was added, followed by a mixture of EtOH:H₂O (9:1, 664 mL) and sodium acetate (31.99 g, 390 mmol) and the mixture was stirred at rt for 2 h. Water (1.5 L) was added and the suspension was filtered and dried under vacuum to afford the title compound (69 g, quant yield).

¹H-NMR (CDCl₃, 300 MHz), δ (ppm): 8.35 (s, 1H), 7.51 (t, J=9.8 Hz, 1H), 6.71 (m, 2H), 4.40 (q, J=7.1 Hz, 2H), 4.01 (q, J=7.1 Hz, 2H), 1.42 (t, J=7.1 Hz, 3H), 1.41 (t, J=7.1 Hz, 3H).
b) Ethyl 4-acetyl-1-(4-ethoxy-2-fluorophenyl)-5-methyl-1H-pyrazole-3-carboxylate: Acetylacetone (17.4 mL, 169 mmol) was added to a solution of sodium ethoxide (21 wt % in ethanol, 63.2 mL, 169 mmol) and the mixture was stirred at rt for 16 h. The compound prepared in step a (48.9 g, 169 mmol) and additional EtOH were added and the mixture was stirred at rt for 4 h and then was let it stand 18 h without stirring. Water (690 mL) was added and the suspension was filtered and dried to afford the title compound (49.5 g, 87% yield).

¹H-NMR (CDCl₃, 300 MHz), δ (ppm): 7.33 (t, J=8.7 Hz, 1H), 6.78 (m, 2H), 4.46 (q, J=7.1 Hz, 2H), 4.08 (q, J=7.1 Hz, 2H), 2.60 (s, 3H), 2.33 (d, J=1.5 Hz, 3H), 1.46 (t, J=7.1 Hz, 3H), 1.43 (t, J=7.1 Hz, 3H).
c) 2-(4-Ethoxy-2-fluorophenyl)-3,4-dimethyl-2H-pyrazolo[3,4-d]pyridazin-7(6H)-one: To a solution of the compound prepared in step b (49.5 g, 148 mmol) in EtOH (285 mL), hydrazine (43.2 mL, 444 mmol) was added and the mixture was refluxed for 5 h. The suspension was cooled to rt, the solid was filtered, washed with cold EtOH and the solid was dried under vacuum to afford the title compound (36.2 g, 81% yield).

¹H-NMR (CDCl₃, 400 MHz), δ (ppm): 9.44 (s, 1H), 7.45 (t, J=8.7 Hz, 1H), 6.85 (ddd, J₁=1.1 Hz, J₂=2.6 Hz, J₃=8.6 Hz, 1H), 6.80 (dd, J₁=2.6 Hz, J₂=11.7 Hz, 1H), 4.12 (q, J=7.1 Hz, 2H), 2.58 (s, 3H), 2.57 (d, J=1.5 Hz, 3H), 1.49 (t, J=7.1 Hz, 3H).
d) Title compound: The compound prepared in step c (36.2 g, 119 mmol) was dissolved in POCl₃ (544 mL) and heated at 100° C. for 3 h. The reaction mixture was concentrated under vacuum, the residue was cooled to 0° C. and basified to pH 8 by carefully addition of ice and 28% NaOH aq solution. The resulting solid was stirred for 2 h, filtered, washed with water and the solid was dried under vacuum to afford the title compound (37.5 g, 98% yield).

¹H-NMR (CDCl₃, 300 MHz), δ (ppm): 7.47 (t, J=8.7 Hz, 1H), 6.89 (ddd, J₁=1.1 Hz, J₂=2.6 Hz, J₃=8.6 Hz, 1H), 6.84 (dd, J₁=2.6 Hz, J₂=11.7 Hz, 1H), 4.13 (q, J=7.1 Hz, 2H), 2.96 (s, 3H), 2.71 (d, J=1.5 Hz, 3H), 1.49 (t, J=7.1 Hz, 3H).

### Ex 1. tert-Butyl 4-(2-(4-ethoxy-2-fluorophenyl)-3,4-dimethyl-2H-pyrazolo[3,4-d]pyridazin-7-yl)-3,6-dihydropyridine-1(2H)-carboxylate

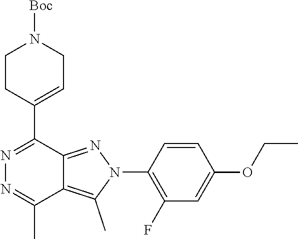 [SMILES: CCOc1ccc(-n2nc3c(C4=CCN(C(=O)OC(C)(C)C)CC4)nnc(C)c3c2C)c(F)c1]

A mixture of 7-chloro-2-(4-ethoxy-2-fluorophenyl)-3,4-dimethyl-2H-pyrazolo[3,4-d]pyridazine (INT 1, 100 mg, 0.312 mmol), N-Boc-1,2,3,6-tetrahydropyridine-4-boronic acid pinacol ester (116 mg, 0.374 mmol) and Na₂CO₃ (66 mg, 0.624 mmol) in a mixture of toluene/EtOH/H₂O (1/0.34/0.34, 2.5 mL) was degassed for 10 min and Pd(PPh₃)₄ (38 mg, 0.033 mmol) was added. The mixture was degassed again for 10 min and heated at 80° C. under argon for 16 h. EtOAc was added, washed with NaHCO₃ sat solution, brine, and the organic layer was concentrated. Purification by flash chromatography, silica gel, gradient from hexane to 100% EtOAc afforded the title compound (116 mg, 80% yield).

¹H-NMR, (CDCl₃, 400 MHz), δ (ppm): 7.69 (m, 1H), 7.46 (t, J=8.6 Hz, 1H), 6.89 (ddd, J₁=1.1 Hz, J₂=2.6 Hz, J₃=8.6 Hz, 1H), 6.84 (dd, J₁=2.6 Hz, J₂=11.7 Hz, 1H), 4.23 (m, 2H), 4.13 (q, J=6.9 Hz, 2H), 3.69 (m, 2H), 2.93 (m, 2H), 2.93 (s, 3H), 2.69 (d, J=1.6 Hz, 3H), 1.50 (t, J=6.9 Hz), 3H), 1.50 (s, 9H).

This method was used for the preparation of Ex 2-11 using suitable starting materials:

| | | | Method/ | |
|---|---|---|---|---|
| | | | Ret | |
| EX | Structure | Chemical name | (min) | MS |
| 2 | 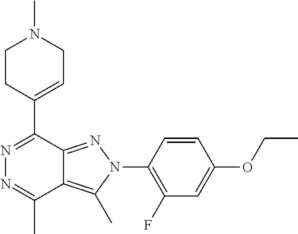 [SMILES: CCOc1ccc(-n2nc3c(C4=CCN(C)CC4)nnc(C)c3c2C)c(F)c1] | 2-(4-Ethoxy-2- fluorophenyl)-3,4- dimethyl-7-(1-methyl- 1,2,3,6- tetrahydropyridin-4-yl)- 2H-pyrazolo[3,4- d]pyridazine | B/3.18 | 382.2 (M + H) |
| 3 | 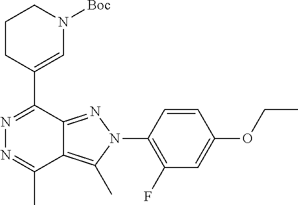 [SMILES: CCOc1ccc(-n2nc3c(C4=CN(C(=O)OC(C)(C)C)CCC4)nnc(C)c3c2C)c(F)c1] | tert-Butyl 5-(2-(4- ethoxy-2- fluorophenyl)-3,4- dimethyl-2H- pyrazolo[3,4- d]pyridazin-7-yl)-3,4- dihydropyridine-1(2H)- carboxylate | | 468.1 (M + H) |
| 4 | 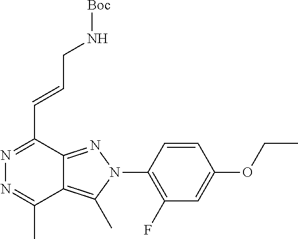 [SMILES: CCOc1ccc(-n2nc3c(/C=C/CNC(=O)OC(C)(C)C)nnc(C)c3c2C)c(F)c1] | tert-Butyl (E)-(3-(2-(4- ethoxy-2- fluorophenyl)-3,4- dimethyl-2H- pyrazolo[3,4- d]pyridazin-7- yl)allyl)carbamate | B/4.29 | 442.2 (M + H) |
| 5 | 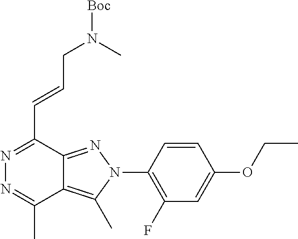 [SMILES: CCOc1ccc(-n2nc3c(/C=C/CN(C)C(=O)OC(C)(C)C)nnc(C)c3c2C)c(F)c1] | tert-Butyl (E)-(3-(2-(4- ethoxy-2- fluorophenyl)-3,4- dimethyl-2H- pyrazolo[3,4- d]pyridazin-7- yl)allyl)(methyl)carbamate | B/4.39 | 456.2 (M + H) |
| 6 | 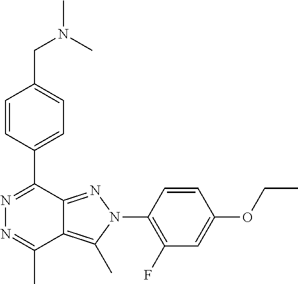 [SMILES: CCOc1ccc(-n2nc3c(-c4ccc(CN(C)C)cc4)nnc(C)c3c2C)c(F)c1] | 1-(4-(2-(4-Ethoxy-2- fluorophenyl)-3,4- dimethyl-2H- pyrazolo[3,4- d]pyridazin-7- yl)phenyl)-N,N- dimethylmethanamine | A/5.00 | 420.2 (M + H) |
| 7 | 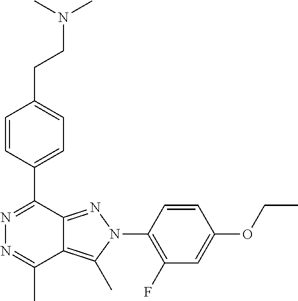 [SMILES: CCOc1ccc(-n2nc3c(-c4ccc(CCN(C)C)cc4)nnc(C)c3c2C)c(F)c1] | 2-(4-(2-(4-Ethoxy-2- fluorophenyl)-3,4- dimethyl-2H- pyrazolo[3,4- d]pyridazin-7- yl)phenyl)-N,N- dimethylethan-1-amine | A/5.05 | 434.1 (M + H) |
| 8 | 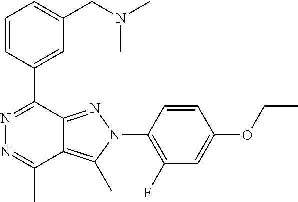 [SMILES: CCOc1ccc(-n2nc3c(-c4cccc(CN(C)C)c4)nnc(C)c3c2C)c(F)c1] | 1-(3-(2-(4-Ethoxy-2- fluorophenyl)-3,4- dimethyl-2H- pyrazolo[3,4- d]pyridazin-7- yl)phenyl)-N,N- dimethylmethanamine | A/5.06 | 420.2 (M + H) |
| 9 | 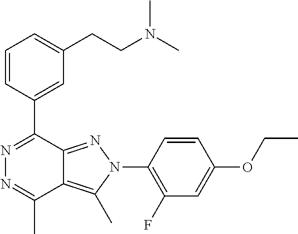 [SMILES: CCOc1ccc(-n2nc3c(-c4cccc(CCN(C)C)c4)nnc(C)c3c2C)c(F)c1] | 2-(3-(2-(4-Ethoxy-2- fluorophenyl)-3,4- dimethyl-2H- pyrazolo[3,4- d]pyridazin-7- yl)phenyl)-N,N- dimethylethan-1-amine | A/5.12 | 434.2 (M + H) |
| 10 | 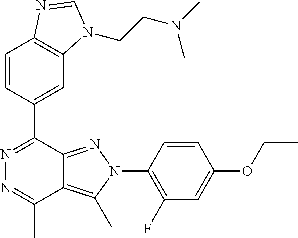 [SMILES: CCOc1ccc(-n2nc3c(-c4ccc5ncn(CCN(C)C)c5c4)nnc(C)c3c2C)c(F)c1] | 2-(6-(2-(4-Ethoxy-2- fluorophenyl)-3,4- dimethyl-2H- pyrazolo[3,4- d]pyridazin-7-yl)-1H- benzo[d]imidazol-1-yl)- N,N-dimethylethan-1- amine | A/4.73 | 474.2 (M + H) |
| 11 | 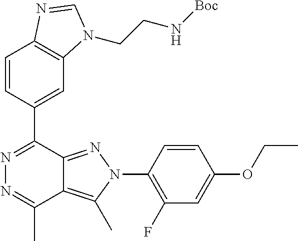 [SMILES: CCOc1ccc(-n2nc3c(-c4ccc5ncn(CCNC(=O)OC(C)(C)C)c5c4)nnc(C)c3c2C)c(F)c1] | tert-Butyl (2-(6-(2-(4- ethoxy-2- fluorophenyl)-3,4- dimethyl-2H- pyrazolo[3,4- d]pyridazin-7-yl)-1H- benzo[d]imidazol-1- yl)ethyl)carbamate | B/3.85 | 546.3 (M + H) |

### Ex 12. tert-Butyl 3-(2-(4-ethoxy-2-fluorophenyl)-3,4-dimethyl-2H-pyrazolo[3,4-d]pyridazin-7-yl)azetidine-1-carboxylate

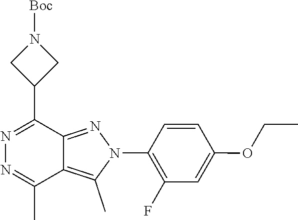 [SMILES: CCOc1ccc(-n2nc3c(C4CN(C(=O)OC(C)(C)C)C4)nnc(C)c3c2C)c(F)c1] [1391] a) (1-(tert-Butoxycarbonyl)azetidin-3-yl)zinc(II) iodide (.LiCl): In a dried flask, anh LiCl (212 mg, 5 mmol) was dried at 150-170° C. under high vacuum for 20 min. Zinc dust (490 mg, 7.49 mmol) was added under Ar and the mixture was dried again at 150-170° C. under high vacuum for 20 min. The reaction flask was evacuated and refilled with argon several times, THF (5 mL), 1,2-dibromoethane (4.3 μL, 0.05 mmol), chlorotrimethylsilane (0.032 mL, 0.25 mmol) and tert-butyl 3-iodoazetidine-1-carboxylate (0.867 mL, 5 mmol) were added and the mixture was stirred at rt for 24 h to afford a solution of the title product that was separated from the remaining zinc powder and used directly in step b.
b) Title compound: In a microwave vial, 7-chloro-2-(4-ethoxy-2-fluorophenyl)-3,4-dimethyl-2H-pyrazolo[3,4-d]pyridazine (60 mg, 0.187 mmol) and Pd(PPh₃)₄ (43 mg, 0.037 mmol) were weighted. A solution of (1-(tert-butoxycarbonyl)azetidin-3-yl)zinc(II) iodide.LiCl in THF (0.5 mL, 0.374 mmol), prepared in step a, was added and the solvent was removed under vacuum. The residue was dissolved in DMF (1.6 mL) and the mixture was irradiated with microwave under argon atmosphere at 160° C. for 40 min. The reaction mixture was cooled at rt, K₂CO₃ 10% solution was added and extracted with EtOAc. The organic layer was washed with brine, dried over Na₂SO₄, filtered and the solvent was removed under vacuum. Purification of the residue by flash chromatography, silica gel, gradient CH to 100% EtOAc afforded the title product (60 mg, 72% yield).

HPLC (Method B): Ret, 4.33 min; ESI⁺-MS m/z, 442.2 (M+H).

### Ex 13. tert-Butyl 4-(2-(4-ethoxy-2-fluorophenyl)-3,4-dimethyl-2H-pyrazolo[3,4-d]pyridazin-7-yl)piperidine-1-carboxylate

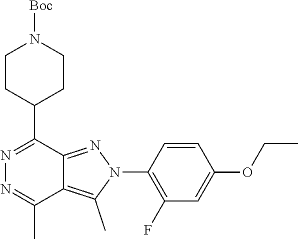 [SMILES: CCOc1ccc(-n2nc3c(C4CCN(C(=O)OC(C)(C)C)CC4)nnc(C)c3c2C)c(F)c1]

A solution of tert-butyl 4-(2-(4-ethoxy-2-fluorophenyl)-3,4-dimethyl-2H-pyrazolo[3,4-d]pyridazin-7-yl)-3,6-dihydropyridine-1(2H)-carboxylate (Ex 1, 112 mg, 0.240 mmol) in MeOH (5 mL) was purged with argon and vacuum. Pd(OH)₂ on carbon 20 wt. % (23 mg) was added, the mixture was purged again with argon and H₂ and then stirred at rt under H₂ atmosphere for 4 h (or until no starting material was present). The reaction mixture was filtered through a plug of Celite and the solvent was removed to afford the title product (100 mg, 89% yield).

HPLC (Method B): Ret, 4.34 min. ESI⁺-MS m/z, 470.2 (M+H).

This method was used for the preparation of Ex 14-17 using suitable starting materials:

| | | | Method/ | |
|---|---|---|---|---|
| | | | Ret | |
| EX | Structure | Chemical name | (min) | MS |
| 14 | 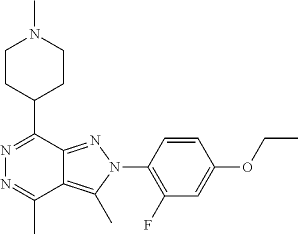 [SMILES: CCOc1ccc(-n2nc3c(C4CCN(C)CC4)nnc(C)c3c2C)c(F)c1] | 2-(4-Ethoxy-2- fluorophenyl)-3,4- dimethyl-7-(1- methylpiperidin-4-yl)- 2H-pyrazolo[3,4- d]pyridazine | A/4.60 | 384.2 (M + H) |
| 15 | 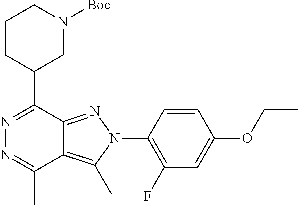 [SMILES: CCOc1ccc(-n2nc3c(C4CCCN(C(=O)OC(C)(C)C)C4)nnc(C)c3c2C)c(F)c1] | tert-Butyl-3-(2-(4- ethoxy-2- fluorophenyl)-3,4- dimethyl-2H- pyrazolo[3,4- d]pyridazin-7- yl)piperidine-1- carboxylate | B/4.46 | 470.2 (M + H) |
| 16 | 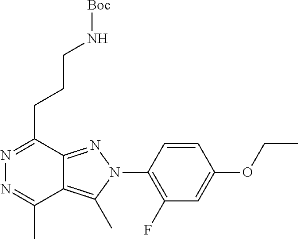 [SMILES: CCOc1ccc(-n2nc3c(CCCNC(=O)OC(C)(C)C)nnc(C)c3c2C)c(F)c1] | tert-Butyl-(3-(2-(4- ethoxy-2- fluorophenyl)-3,4- dimethyl-2H- pyrazolo[3,4- d]pyridazin-7- yl)propyl)carbamate | B/4.17 | 444.2 (M + H) |
| 17 | 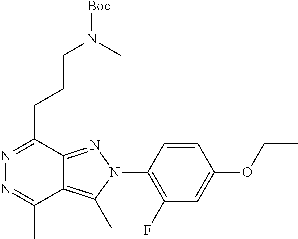 [SMILES: CCOc1ccc(-n2nc3c(CCCN(C)C(=O)OC(C)(C)C)nnc(C)c3c2C)c(F)c1] | tert-Butyl-(3-(2-(4- ethoxy-2- fluorophenyl)-3,4- dimethyl-2H- pyrazolo[3,4- d]pyridazin-7- yl)propyl)(methyl)carba- mate | B/4.28 | 458.26 (M + H) |

### Ex 18. 2-(4-Ethoxy-2-fluorophenyl)-3,4-dimethyl-7-(piperidin-4-yl)-2H-pyrazolo[3,4-d]pyridazine

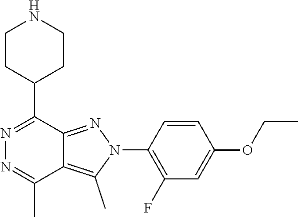 [SMILES: CCOc1ccc(-n2nc3c(C4CCNCC4)nnc(C)c3c2C)c(F)c1]

tert-Butyl 4-(2-(4-ethoxy-2-fluorophenyl)-3,4-dimethyl-2H-pyrazolo[3,4-d]pyridazin-7-yl) piperidine-1-carboxylate (Ex 13, 300 mg, 0.639 mmol) was dissolved in a 4 M HCl solution in dioxane (3.19 mL, 12.78 mmol) at 0° C. and the mixture was stirred at rt for 4 h. The solvent was removed under vacuum to afford the title product (259 mg, quant).

HPLC (Method A): Ret, 4.65 min; ESI⁺-MS m/z, 370.20 (M+H).

This method was used for the preparation of Ex 19-24, using suitable starting materials:

| | | | Method/ | |
|---|---|---|---|---|
| | | | Ret | |
| EX | Structure | Chemical name | (min) | MS |
| 19 | 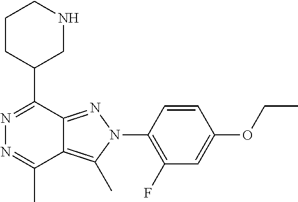 [SMILES: CCOc1ccc(-n2nc3c(C4CCCNC4)nnc(C)c3c2C)c(F)c1] | 2-(4-Ethoxy-2- fluorophenyl)-3,4- dimethyl-7-(piperidin- 3-yl)-2H-pyrazolo[3,4- d]pyridazine | A/4.63 | 392.2 (M + Na) |
| 20 | 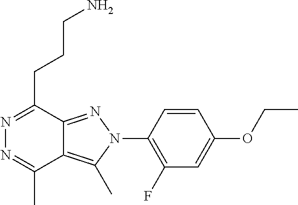 [SMILES: CCOc1ccc(-n2nc3c(CCCN)nnc(C)c3c2C)c(F)c1] | 3-(2-(4-Ethoxy-2- fluorophenyl)-3,4- dimethyl-2H- pyrazolo[3,4- d]pyridazin-7- yl)propan-1-amine | A/4.51 | 344.2 (M + H) |
| 21 | 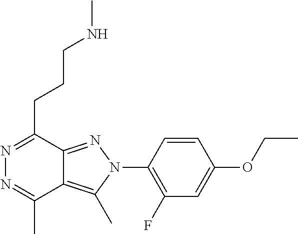 [SMILES: CCOc1ccc(-n2nc3c(CCCNC)nnc(C)c3c2C)c(F)c1] | 3-(2-(4-Ethoxy-2- fluorophenyl)-3,4- dimethyl-2H- pyrazolo[3,4- d]pyridazin-7-yl)-N- methylpropan-1-amine | | 358.1 (M + H) |
| 22 | 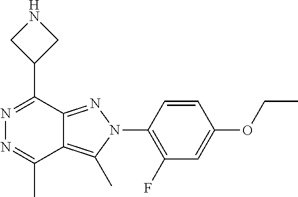 [SMILES: CCOc1ccc(-n2nc3c(C4CNC4)nnc(C)c3c2C)c(F)c1] | 7-(Azetidin-3-yl)-2-(4- ethoxy-2- fluorophenyl)-3,4- dimethyl-2H- pyrazolo[3,4- d]pyridazine | B/2.98 | 342.1 (M + H) |
| 23 | 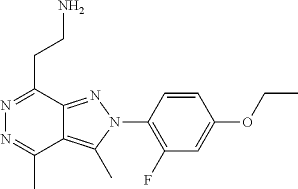 [SMILES: CCOc1ccc(-n2nc3c(CCN)nnc(C)c3c2C)c(F)c1] | 2-(2-(4-Ethoxy-2- fluorophenyl)-3,4- dimethyl-2H- pyrazolo[3,4- d]pyridazin-7-yl)ethan- 1-amine | | 329.3 (M + H) |
| 24 | 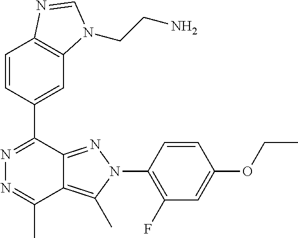 [SMILES: CCOc1ccc(-n2nc3c(-c4ccc5ncn(CCN)c5c4)nnc(C)c3c2C)c(F)c1] | 2-(6-(2-(4-Ethoxy-2- fluorophenyl)-3,4- dimethyl-2H- pyrazolo[3,4- d]pyridazin-7-yl)-1H- benzo[d]imidazol-1- yl)ethan-1-amine | A/4.63 | 446.2 (M + H) |

### Ex 25. 2-(4-Ethoxy-2-fluorophenyl)-3,4-dimethyl-7-(1-phenethylpiperidin-4-yl)-2H-pyrazolo[3,4-d]pyridazine

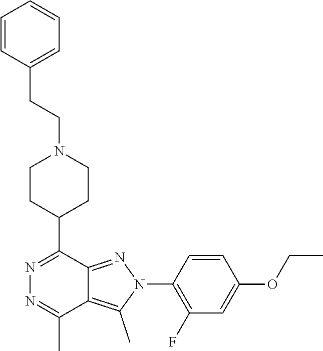 [SMILES: CCOc1ccc(-n2nc3c(C4CCN(CCc5ccccc5)CC4)nnc(C)c3c2C)c(F)c1]

To a solution of 2-(4-ethoxy-2-fluorophenyl)-3,4-dimethyl-7-(piperidin-4-yl)-2H-pyrazolo[3,4-d]pyridazine hydrochloride (Ex 18, 60 mg, 0.147 mmol) in DCE (5 mL), DIPEA (0.109 mL, 0.627 mmol) was added and the mixture was stirred at rt for 10 min. 2-Phenylacetaldehyde (0.017 mL, 0.150 mmol) and NaBH(OAc)₃ (40 mg, 0.188 mmol) were added and the reaction mixture was stirred at rt for 2 days. DCM was added, washed with NaHCO₃ sat solution, brine and the organic layer was concentrated under vacuum. Purification of the residue by flash chromatography, silica gel, gradient DCM to 20% MeOH afforded the title product (40 mg, 57% yield).

HPLC (Method A): Ret, 5.39 min; ESI⁺-MS m/z, 474.3 (M+H).

This method was used for the preparation of Ex 26-41 using suitable starting materials:

| | | | Method/ | |
|---|---|---|---|---|
| | | | Ret | |
| EX | Structure | Chemical name | (min) | MS |
| 26 | 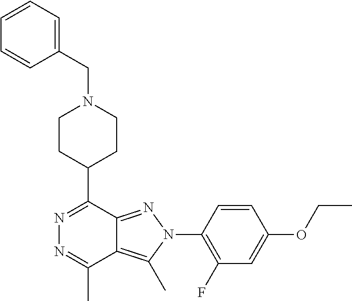 [SMILES: CCOc1ccc(-n2nc3c(C4CCN(Cc5ccccc5)CC4)nnc(C)c3c2C)c(F)c1] | 7-(1-Benzylpiperidin-4- yl)-2-(4-ethoxy-2- fluorophenyl)-3,4- dimethyl-2H- pyrazolo[3,4- d]pyridazine | A/5.21 | 460.2 (M + H) |
| 27 | 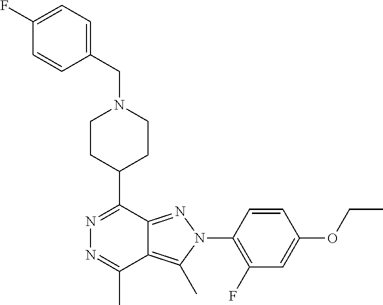 [SMILES: CCOc1ccc(-n2nc3c(C4CCN(Cc5ccc(F)cc5)CC4)nnc(C)c3c2C)c(F)c1] | 2-(4-Ethoxy-2- fluorophenyl)-7-(1-(4- fluorobenzyl)piperidin- 4-yl)-3,4-dimethyl-2H- pyrazolo[3,4- d]pyridazine | A/5.30 | 478.2 (M + H) |
| 28 | 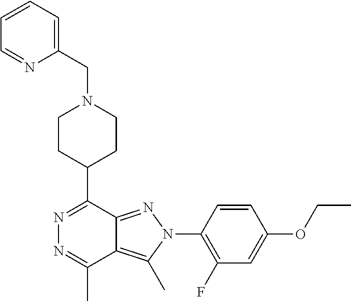 [SMILES: CCOc1ccc(-n2nc3c(C4CCN(Cc5ccccn5)CC4)nnc(C)c3c2C)c(F)c1] | 2-(4-Ethoxy-2- fluorophenyl)-3,4- dimethyl-7-(1-(pyridin- 2-ylmethyl)piperidin-4- yl)-2H-pyrazolo[3,4- d]pyridazine | A/4.87 | 461.2 (M + H) |
| 29 | 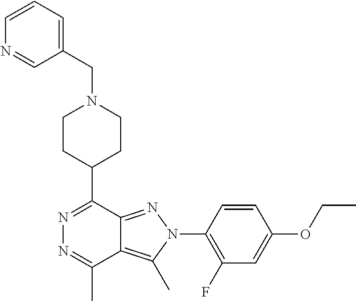 [SMILES: CCOc1ccc(-n2nc3c(C4CCN(Cc5cccnc5)CC4)nnc(C)c3c2C)c(F)c1] | 2-(4-Ethoxy-2- fluorophenyl)-3,4- dimethyl-7-(1-(pyridin- 3-ylmethyl)piperidin-4- yl)-2H-pyrazolo[3,4- d]pyridazine | A/4.50 | 461.2 (M + H) |
| 30 | 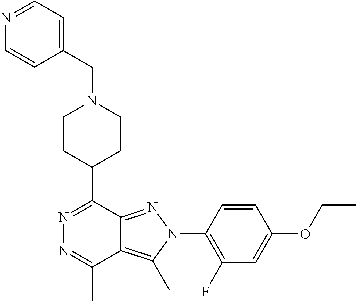 [SMILES: CCOc1ccc(-n2nc3c(C4CCN(Cc5ccncc5)CC4)nnc(C)c3c2C)c(F)c1] | 2-(4-Ethoxy-2- fluorophenyl)-3,4- dimethyl-7-(1-(pyridin- 4-ylmethyl)piperidin-4- yl)-2H-pyrazolo[3,4- d]pyridazine | A/4.41 | 461.2 (M + H) |
| 31 | 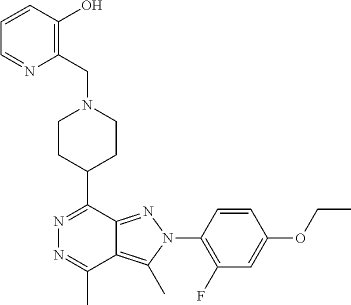 [SMILES: CCOc1ccc(-n2nc3c(C4CCN(Cc5ncccc5O)CC4)nnc(C)c3c2C)c(F)c1] | 2-((4-(2-(4-Ethoxy-2- fluorophenyl)-3,4- dimethyl-2H- pyrazolo[3,4- d]pyridazin-7- yl)piperidin-1- yl)methyl)pyridin-3-ol | A/4.82 | 477.2 (M + H) |
| 32 | 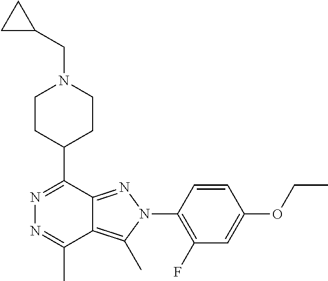 [SMILES: CCOc1ccc(-n2nc3c(C4CCN(CC5CC5)CC4)nnc(C)c3c2C)c(F)c1] | 7-(1-(Cyclopropyl- methyl)piperidin-4-yl)- 2-(4-ethoxy-2- fluorophenyl)-3,4- dimethyl-2H- pyrazolo[3,4- d]pyridazine | A/4.92 | 424.2 (M + H) |
| 33 | 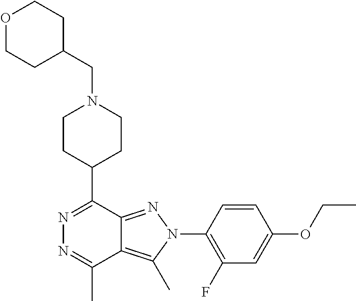 [SMILES: CCOc1ccc(-n2nc3c(C4CCN(CC5CCOCC5)CC4)nnc(C)c3c2C)c(F)c1] | 2-(4-Ethoxy-2- fluorophenyl)-3,4- dimethyl-7-(1- ((tetrahydro-2H-pyran- 4-yl)methyl)piperidin-4- yl)-2H-pyrazolo[3,4- d]pyridazine | A/4.78 | 468.3 (M + H) |
| 34 | 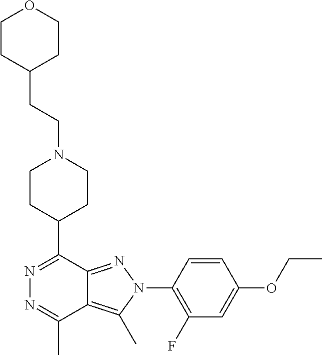 [SMILES: CCOc1ccc(-n2nc3c(C4CCN(CCC5CCOCC5)CC4)nnc(C)c3c2C)c(F)c1] | 2-(4-Ethoxy-2- fluorophenyl)-3,4- dimethyl-7-(1-(2- (tetrahydro-2H-pyran- 4-yl)ethyl)piperidin-4- yl)-2H-pyrazolo[3,4- d]pyridazine | A/4.91 | 482.3 (M + H) |
| 35 | 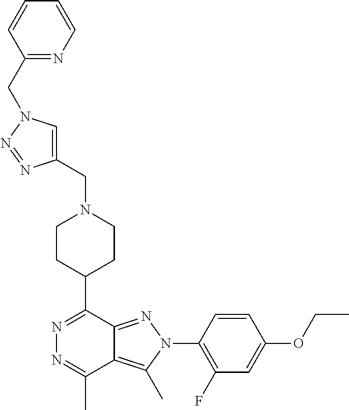 [SMILES: CCOc1ccc(-n2nc3c(C4CCN(Cc5cn(Cc6ccccn6)nn5)CC4)nnc(C)c3c2C)c(F)c1] | 2-(4-Ethoxy-2- fluorophenyl)-3,4- dimethyl-7-(1-((1- (pyridin-2-ylmethyl)- 1H-1,2,3-triazol-4- yl)methyl)piperidin-4- yl)-2H-pyrazolo[3,4- d]pyridazine | A/4.85 | 542.3 (M + H) |
| 36 | 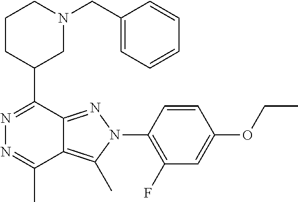 [SMILES: CCOc1ccc(-n2nc3c(C4CCCN(Cc5ccccc5)C4)nnc(C)c3c2C)c(F)c1] | 7-(1-Benzylpiperidin-3- yl)-2-(4-ethoxy-2- fluorophenyl)-3,4- dimethyl-2H- pyrazolo[3,4- d]pyridazine | A/5.19 | 460.2 (M + H) |
| 37 | 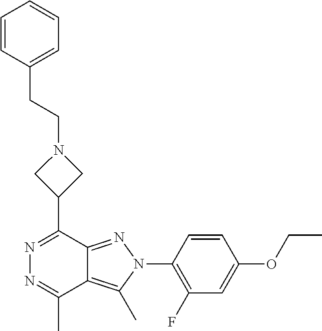 [SMILES: CCOc1ccc(-n2nc3c(C4CN(CCc5ccccc5)C4)nnc(C)c3c2C)c(F)c1] | 2-(4-Ethoxy-2- fluorophenyl)-3,4- dimethyl-7-(1- phenethylazetidin-3- yl)-2H-pyrazolo[3,4- d]pyridazine | A/5.32 | 446.2 (M + H) |
| 38 | 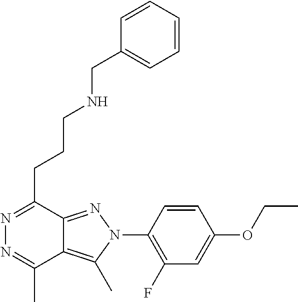 [SMILES: CCOc1ccc(-n2nc3c(CCCNCc4ccccc4)nnc(C)c3c2C)c(F)c1] | N-Benzyl-3-(2-(4- ethoxy-2- fluorophenyl)-3,4- dimethyl-2H- pyrazolo[3,4- d]pyridazin-7- yl)propan-1-amine | A/5.28 | 434.2 (M + H) |
| 39 | 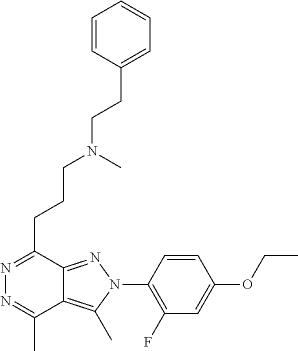 [SMILES: CCOc1ccc(-n2nc3c(CCCN(C)CCc4ccccc4)nnc(C)c3c2C)c(F)c1] | 3-(2-(4-Ethoxy-2- fluorophenyl)-3,4- dimethyl-2H- pyrazolo[3,4- d]pyridazin-7-yl)-N- methyl-N- phenethylpropan-1- amine | A/5.49 | 462.2 (M + H) |
| 40 | 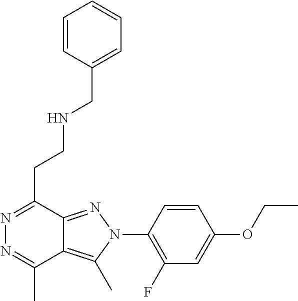 [SMILES: CCOc1ccc(-n2nc3c(CCNCc4ccccc4)nnc(C)c3c2C)c(F)c1] | N-Benzyl-2-(2-(4- ethoxy-2- fluorophenyl)-3,4- dimethyl-2H- pyrazolo[3,4- d]pyridazin-7-yl)ethan- 1-amine | A/5.07 | 420.2 (M + H) |
| 41 | 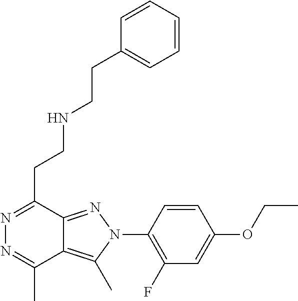 [SMILES: CCOc1ccc(-n2nc3c(CCNCCc4ccccc4)nnc(C)c3c2C)c(F)c1] | 2-(2-(4-Ethoxy-2- fluorophenyl)-3,4- dimethyl-2H- pyrazolo[3,4- d]pyridazin-7-yl)-N- phenethylethan-1- amine | A/5.20 | 434.2 (M + H) |

### Ex 42: 2-(4-Ethoxy-2-fluorophenyl)-3,4-dimethyl-7-(1-(2-(pyridin-2-yl)ethyl)piperidin-4-yl)-2H-pyrazolo[3,4-d]pyridazine

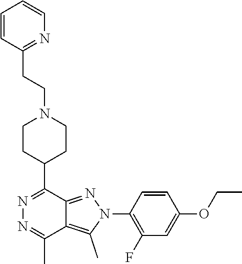 [SMILES: CCOc1ccc(-n2nc3c(C4CCN(CCc5ccccn5)CC4)nnc(C)c3c2C)c(F)c1]

To a solution of 2-(4-ethoxy-2-fluorophenyl)-3,4-dimethyl-7-(piperidin-4-yl)-2H-pyrazolo[3,4-d]pyridazine hydrochloride (Ex 18, 62 mg, 0.152 mmol) in ACN (2 mL), DIPEA (0.103 mL, 0.591 mmol) was added and the mixture was stirred at rt for 10 min. 2-(2-Bromoethyl)pyridine (28 mg, 0.152 mmol) and K₂CO₃ (25 mg, 0.177 mmol) were added and the reaction mixture was stirred at rt for 2 days. DCM was added, washed with NaHCO₃ sat solution, brine and the organic layer was concentrated under vacuum. Purification of the residue by flash chromatography, silica gel, gradient DCM to 20% MeOH afforded the title product (23 mg, 32% yield).

HPLC (Method A): Ret, 4.54 min; ESI⁺-MS m/z, 475.25 (M+H).

This method was used for the preparation of Ex 43-56 using suitable starting materials:

| | | | Method/ | |
|---|---|---|---|---|
| | | | Ret | |
| EX | Structure | Chemical name | (min) | MS |
| 43 | 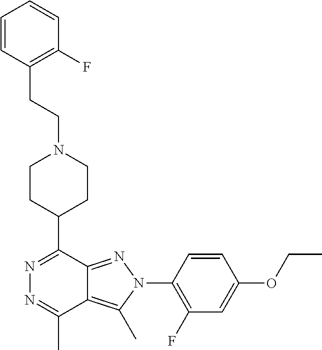 [SMILES: CCOc1ccc(-n2nc3c(C4CCN(CCc5ccccc5F)CC4)nnc(C)c3c2C)c(F)c1] | 2-(4-Ethoxy-2- fluorophenyl)-7-(1-(2- flurophenethyl)piperidin- 4-yl)-3,4-dimethyl- 2H-pyrazolo[3,4- d]pyridazine | A/5.42 | 492.2 (M + H) |
| 44 | 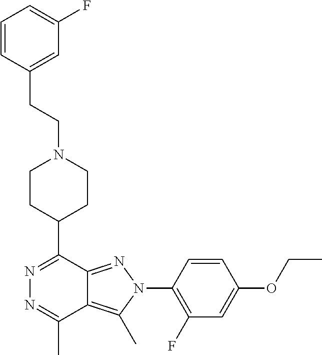 [SMILES: CCOc1ccc(-n2nc3c(C4CCN(CCc5cccc(F)c5)CC4)nnc(C)c3c2C)c(F)c1] | 2-(4-Ethoxy-2- fluorophenyl)-7-(1-(3- fluorophenethyl)piperidin- 4-yl)-3,4-dimethyl- 2H-pyrazolo[3,4- d]pyridazine | A/5.49 | 492.2 (M + H) |
| 45 | 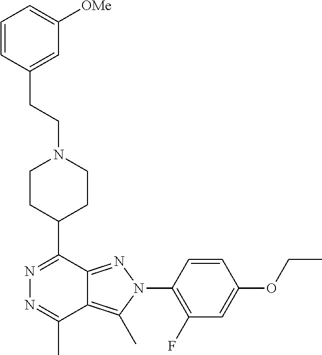 [SMILES: CCOc1ccc(-n2nc3c(C4CCN(CCc5cccc(OC)c5)CC4)nnc(C)c3c2C)c(F)c1] | 2-(4-Ethoxy-2- fluorophenyl)-7-(1-(3- methoxyphenethyl) piperidin-4-yl)-3,4- dimethyl-2H- pyrazolo[3,4- d]pyridazine | A/5.42 | 504.3 (M + H) |
| 46 | 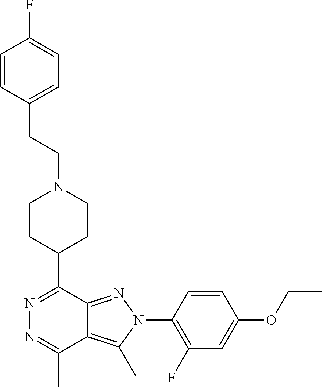 [SMILES: CCOc1ccc(-n2nc3c(C4CCN(CCc5ccc(F)cc5)CC4)nnc(C)c3c2C)c(F)c1] | 2-(4-Ethoxy-2- fluorophenyl)-7-(1-(4- fluorophenethyl)piperidin- 4-yl)-3,4-dimethyl- 2H-pyrazolo[3,4- d]pyridazine | A/5.46 | 492.2 (M + H) |
| 47 | 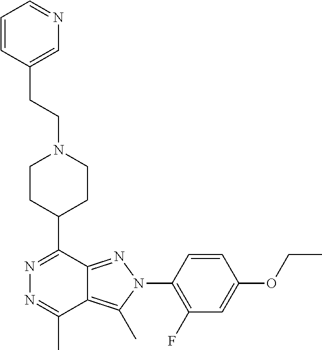 [SMILES: CCOc1ccc(-n2nc3c(C4CCN(CCc5cccnc5)CC4)nnc(C)c3c2C)c(F)c1] | 2-(4-Ethoxy-2- fluorophenyl)-3,4- dimethyl-7-(1-(2- (pyridin-3- yl)ethyl)piperidin-4-yl)- 2H-pyrazolo[3,4- d]pyridazine | A/4.33 | 475.3 (M + H) |
| 48 | 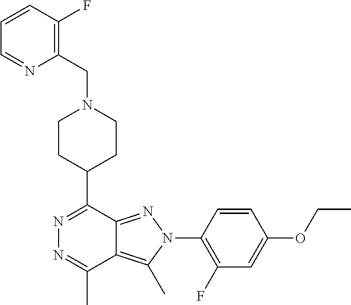 [SMILES: CCOc1ccc(-n2nc3c(C4CCN(Cc5ncccc5F)CC4)nnc(C)c3c2C)c(F)c1] | 2-(4-Ethoxy-2- fluorophenyl)-7-(1-((3- fluoropyridin-2- yl)methyl)piperidin-4- yl)-3,4-dimethyl-2H- pyrazolo[3,4- d]pyridazine | A/4.68 | 479.2 (M + H) |
| 49 | 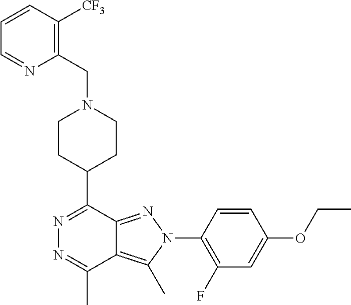 [SMILES: CCOc1ccc(-n2nc3c(C4CCN(Cc5ncccc5C(F)(F)F)CC4)nnc(C)c3c2C)c(F)c1] | 2-(4-Ethoxy-2- fluorophenyl)-3,4- dimethyl-7-(1-((3- (trifluoromethyl)pyridin- 2-yl)methyl)piperidin-4- yl)-2H-pyrazolo[3,4- d]pyridazine | A/5.32 | 529.2 (M + H) |
| 50 | 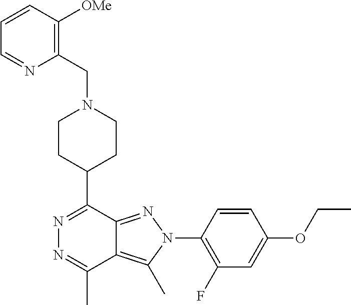 [SMILES: CCOc1ccc(-n2nc3c(C4CCN(Cc5ncccc5OC)CC4)nnc(C)c3c2C)c(F)c1] | 2-(4-Ethoxy-2- fluorophenyl)-7-(1-((3- methoxypyridin-2- yl)methyl)piperidin-4- yl)-3,4-dimethyl-2H- pyrazolo[3,4- d]pyridazine | A/4.98 | 491.2 (M + H) |
| 51 | 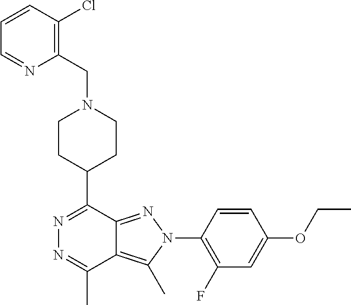 [SMILES: CCOc1ccc(-n2nc3c(C4CCN(Cc5ncccc5Cl)CC4)nnc(C)c3c2C)c(F)c1] | 7-(1-((3-Chloropyridin- 2-yl)methyl)piperidin-4- yl)-2-(4-ethoxy-2- fluorophenyl)-3,4- dimethyl-2H- pyrazolo[3,4- d]pyridazine | A/5.11 | 495.2 (M + H) |
| 52 | 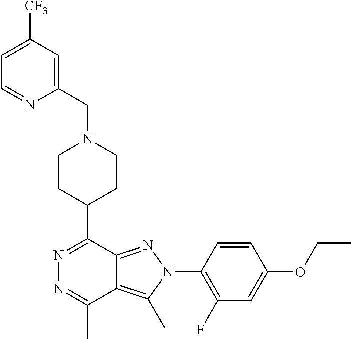 [SMILES: CCOc1ccc(-n2nc3c(C4CCN(Cc5cc(C(F)(F)F)ccn5)CC4)nnc(C)c3c2C)c(F)c1] | 2-(4-Ethoxy-2- fluorophenyl)-3,4- dimethyl-7-(1-((4- (trifluoromethyl)pyridin- 2-yl)methyl)piperidin-4- yl)-2H-pyrazolo[3,4- d]pyridazine | A/5.37 | 529.2 (M + H) |
| 53 | 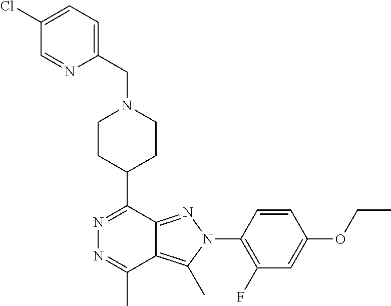 [SMILES: CCOc1ccc(-n2nc3c(C4CCN(Cc5ccc(Cl)cn5)CC4)nnc(C)c3c2C)c(F)c1] | 7-(1-((5-Chloropyridin- 2-yl)methyl)piperidin-4- yl)-2-(4-ethoxy-2- fluorophenyl)-3,4- dimethyl-2H- pyrazolo[3,4- d]pyridazine | A/5.17 | 495.2 (M + H) |
| 54 | 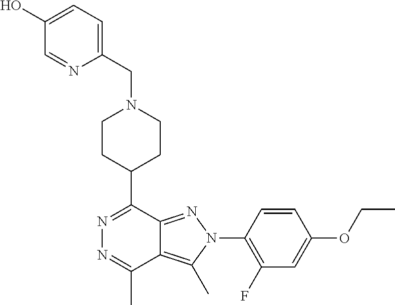 [SMILES: CCOc1ccc(-n2nc3c(C4CCN(Cc5ccc(O)cn5)CC4)nnc(C)c3c2C)c(F)c1] | 6-((4-(2-(4-Ethoxy-2- fluorophenyl)-3,4- dimethyl-2H- pyrazolo[3,4- d]pyridazin-7- yl)piperidin-1- yl)methyl)pyridin-3-ol | A/4.74 | 477.2 (M + H) |
| 55 | 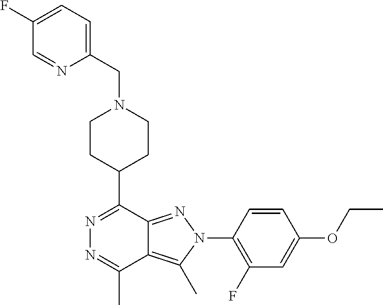 [SMILES: CCOc1ccc(-n2nc3c(C4CCN(Cc5ccc(F)cn5)CC4)nnc(C)c3c2C)c(F)c1] | 2-(4-Ethoxy-2- fluorophenyl)-7-(1-((5- fluoropyridin-2- yl)methyl)piperidin-4- yl)-3,4-dimethyl-2H- pyrazolo[3,4- d]pyridazine | A/4.96 | 479.2 (M + H) |
| 56 | 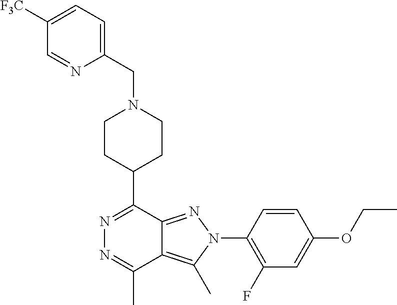 [SMILES: CCOc1ccc(-n2nc3c(C4CCN(Cc5ccc(C(F)(F)F)cn5)CC4)nnc(C)c3c2C)c(F)c1] | 2-(4-Ethoxy-2- fluorophenyl)-3,4- dimethyl-7-(1-((5- (trifluoromethyl)pyridin- 2-yl)methyl)piperidin-4- yl)-2H-pyrazolo[3,4- d]pyridazine | A/4.91 | 529.2 (M + H) |

### Ex 57: 2-(4-Ethoxy-2-fluorophenyl)-3,4-dimethyl-7-(1-(2-(pyridin-4-yl)ethyl)piperidin-4-yl)-2H-pyrazolo[3,4-d]pyridazine

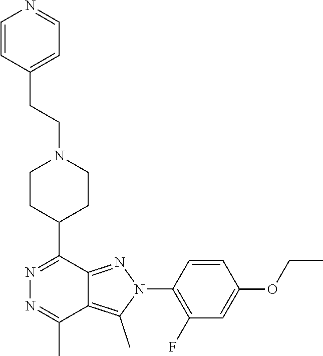 [SMILES: CCOc1ccc(-n2nc3c(C4CCN(CCc5ccncc5)CC4)nnc(C)c3c2C)c(F)c1]

To a solution of 2-(4-ethoxy-2-fluorophenyl)-3,4-dimethyl-7-(piperidin-4-yl)-2H-pyrazolo[3,4-d]pyridazine (Ex 18, 40 mg, 0.11 mmol) in 2-methoxyethanol (1.1 mL) under argon atmosphere, 4-vinylpyridine (20 μl, 0.18 mmol) was added and the reaction mixture was heated at 120° C. in a sealed tube for 24 h. Purification of the residue by flash chromatography, silica gel with 2.5% Et₃N, gradient DCM to 40% MeOH afforded the title compound (26 mg, 50% yield). HPLC (Method A): Ret, 4.30 min; ESI⁺-MS m/z, 475.2 (M+H).

### Ex 58: 2-(4-(2-(4-Ethoxy-2-fluorophenyl)-3,4-dimethyl-2H-pyrazolo[3,4-d]pyridazin-7-yl)piperidin-1-yl)-1-phenylethan-1-ol

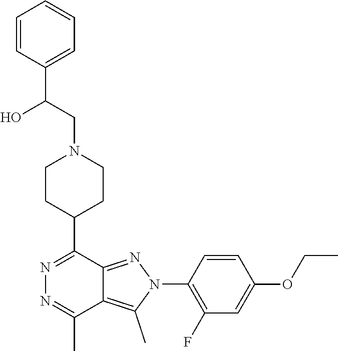 [SMILES: CCOc1ccc(-n2nc3c(C4CCN(CC(O)c5ccccc5)CC4)nnc(C)c3c2C)c(F)c1]

To a solution of 2-(4-ethoxy-2-fluorophenyl)-3,4-dimethyl-7-(piperidin-4-yl)-2H-pyrazolo[3,4-d]pyridazine (Ex 18, 50 mg, 0.135 mmol) in ACN (1.6 mL), K₂CO₃ (56 mg, 0.406 mmol) and 2-bromoacetophenone (32 mg, 0.162 mmol) were added and the mixture was stirred under argon at rt for 2.5 h. The mixture was concentrated, diluted with MeOH (1 mL) and NaBH₄ (13 mg, 0.338 mmol) was slowly added. The reaction mixture was stirred for 2 h at rt, water was added and the organic solvent was removed under vacuum. The residue was extracted with EtOAc, dried over Na₂SO₄ and concentrated. Purification of the residue by flash chromatography, silica gel, gradient DCM to 10% MeOH afforded the title compound (12 mg, 18% yield).

HPLC (Method A): Ret, 5.18 min; ESI⁺-MS m/z, 490.2 (M+H).

### Ex 59: 2-(4-Ethoxy-2-fluorophenyl)-3,4-dimethyl-2H-pyrazolo[3,4-d]pyridazine-7-carbonitrile

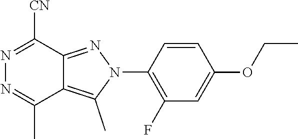 [SMILES: CCOc1ccc(-n2nc3c(C#N)nnc(C)c3c2C)c(F)c1]

A solution of 7-chloro-2-(4-ethoxy-2-fluorophenyl)-3,4-dimethyl-2H-pyrazolo[3,4-d]pyridazine (INT 1, 200 mg, 0.624 mmol), Zn(CN)₂ (132 mg, 1.122 mmol) and Pd(PPh₃)₄ (144 mg, 0.125 mmol) in DMF (4 mL) was irradiated with microwaves at 160° C. for 40 min. The reaction mixture was cooled at rt, K₂CO₃ 10% solution was added and extracted with EtOAc. The organic layer was washed with brine, dried over Na₂SO₄, filtered and concentrated. Purification of the residue by flash chromatography, silica gel, gradient DCM to 5% MeOH, afforded the title compound (145 mg, 74% yield).

HPLC (Method B): Ret, 4.22 min; ESI⁺-MS m/z, 312.1 (M+H).

### Ex 60: 3-(1-((2-(4-Ethoxy-2-fluorophenyl)-3,4-dimethyl-2H-pyrazolo[3,4-d]pyridazin-7-yl)methyl)piperidin-4-yl)phenol

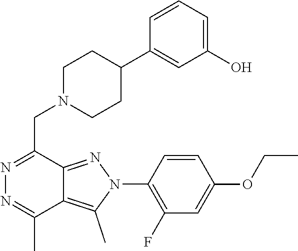 [SMILES: CCOc1ccc(-n2nc3c(CN4CCC(c5cccc(O)c5)CC4)nnc(C)c3c2C)c(F)c1] [1419] a) Ethyl 2-(4-ethoxy-2-fluorophenyl)-3,4-dimethyl-2H-pyrazolo[3,4-d]pyridazine-7-carboxylate.

To a solution of 2-(4-ethoxy-2-fluorophenyl)-3,4-dimethyl-2H-pyrazolo[3,4-d]pyridazine-7-carbonitrile (Ex 59, 240 mg, 0.771 mmol) in EtOH (8 mL) cooled at 0° C., 1.25 M HCl solution in EtOH (9.25 mL, 11.56 mmol) and 4 M HCl solution in dioxane (2.89 mL) were added and the mixture was warmed at rt and then heated at 100° C. for 16 h. The reaction mixture was cooled at 0° C., K₂CO₃ sat solution was added and the mixture was extracted with DCM. The organic layer was dried over Na₂SO₄, filtered and concentrated. Purification of the residue by flash chromatography, silica gel, gradient DCM to 5% MeOH, afforded the title compound (250 mg, 90% yield).

¹H-NMR, (CDCl₃, 400 MHz), δ (ppm): 7.51 (t, J=8.6 Hz, 1H), 6.88 (ddd, J₁=1.1 Hz, J₂=2.6 Hz, J₃=8.6 Hz ,1H), 6.82 (dd, J₁=2.6 Hz, J₂=11.7 Hz, 1H), 4.59 (q, J=6.9 Hz, 2H), 4.13 (q, J=6.9 Hz, 2H), 3.03 (s, 3H), 2.74 (d, J=1.6 Hz, 3H), 1.50 (m, 6H).
b) 2-(4-Ethoxy-2-fluorophenyl)-3,4-dimethyl-2H-pyrazolo[3,4-d]pyridazine-7-carbaldehyde: To a solution of the compound prepared in step a (66 mg, 0.184 mmol) in DCE (5 mL) cooled at 0° C. under argon, diisobutylaluminium hydride (1 M solution in DCM, 368 μl, 0.368 mmol) was added dropwise and the mixture was stirred at 0° C. for 2 h. MeOH was added at 0° C., the reaction mixture was filtered through a plug of Celite and the solvent was removed under vacuum to afford the title compound, that was used in the next step without further purification.
c)Title compound: To a solution of the compound prepared in step b (40 mg, 0.089 mmol) in DCE (5 mL), 3-(piperidin-4-yl)phenol (16 mg, 0.089 mmol) and NaBH(OAc)₃ (38 mg. 0.178 mmol) were added and the mixture was stirred at rt for 16 h. DCM was added, washed with NaHCO₃ sat solution, brine and the organic layer was dried over Na₂SO₄, filtered and concentrated.

Purification of the residue by flash chromatography, silica gel, gradient DCM to 10% MeOH afforded the title product (15 mg, 35% yield).

¹H-NMR, (CDCl₃, 400 MHz), δ (ppm): 7.45 (t, J=8.6 Hz, 1H), 7.11 (t, J=7.8 Hz, 1H), 6.94 (m, 1H), 6.88 (m, 1H), 6.83 (m, 1H), 6.70 (m, 2H), 4.26 (s, 2H), 4.12 (q, J=6.9 Hz, 2H), 3.34 (m, 2H), 2.95 (s, 3H), 2.69 (d, J=1.6 Hz, 3H), 2.46 (m, 1H), 2.40 (m, 2H), 1.82 (m, 4H), 1.49 (t, J=6.9 Hz, 3H).

### Ex 61: tert-Butyl 2-cyano-2-(2-(4-ethoxy-2-fluorophenyl)-3,4-dimethyl-2H-pyrazolo[3,4-d]pyridazin-7-yl)acetate

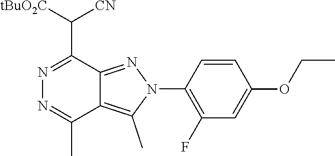 [SMILES: CCOc1ccc(-n2nc3c(C(C#N)C(=O)OC(C)(C)C)nnc(C)c3c2C)c(F)c1]

To a solution of 7-chloro-2-(4-ethoxy-2-fluorophenyl)-3,4-dimethyl-2H-pyrazolo[3,4-d]pyridazine (INT 1, 0.54 g, 1.7 mmol) and Cs₂CO₃ (1.6 g, 5.1 mmol) in anh DMSO (15 mL) under argon, tert-butyl 2-cyanoacetate (0.73 mL, 5.1 mmol) was added and the mixture was irradiated with microwaves at 150° C. for 50 min. The reaction mixture was cooled to rt, EtOAc was added and washed with water and brine. The organic phase was dried with Na₂SO₄, filtered and concentrated under vacuum. Purification of the residue by flash chromatography, silica gel, gradient hexane to 100% acetone afforded the title compound (0.58 g, 80% yield).

HPLC (Method A): Ret, 8.83 min; ESI⁺-MS m/z, 426.2 (M+H).

### Ex 62: 2-(2-(4-Ethoxy-2-fluorophenyl)-3,4-dimethyl-2H-pyrazolo[3,4-d]pyridazin-7-yl)acetonitrile

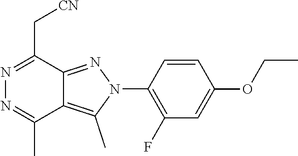 [SMILES: CCOc1ccc(-n2nc3c(CC#N)nnc(C)c3c2C)c(F)c1]

A solution of tert-butyl 2-cyano-2-(2-(4-ethoxy-2-fluorophenyl)-3,4-dimethyl-2H-pyrazolo[3,4-d]pyridazin-7-yl)acetate (Ex 61, 500 mg, 1.1 mmol) in hexafluoro-2-isopropanol (11 mL) was irradiated with microwaves at 160° C. for 60 min. The reaction mixture was cooled at rt and the solvent was removed under vacuum. Purification of the residue by flash chromatography, silica gel, gradient DCM to 30% MeOH afforded the title compound (340 mg, 90% yield).

HPLC (Method A): Ret, 5.57 min; ESI⁺-MS m/z, 326.1 (M+H).

### Ex 63: tert-Butyl (2-(2-(4-ethoxy-2-fluorophenyl)-3,4-dimethyl-2H-pyrazolo[3,4-d]pyridazin-7-yl)ethyl)carbamate

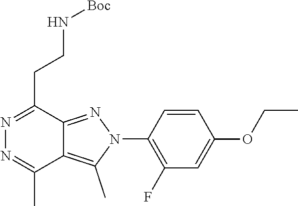 [SMILES: CCOc1ccc(-n2nc3c(CCNC(=O)OC(C)(C)C)nnc(C)c3c2C)c(F)c1]

To a solution of 2-(2-(4-ethoxy-2-fluorophenyl)-3,4-dimethyl-2H-pyrazolo[3,4-d]pyridazin-7-yl)acetonitrile (Ex 62, 307 mg, 0.944 mmol) and NiCl₂.6H₂O (45 mg, 0.189 mmol) in MeOH (5 mL), a solution of di-tert-butyl dicarbonate (515 mg, 2.359 mmol) in MeOH (5 mL) was added and the mixture was cooled at 0° C. NaBH₄ (250 mg, 6.61 mmol) was added in portions at 0° C. and stirred at rt for 1 h; then, the reaction mixture was heated at 80° C. for 16 h. The reaction mixture was cooled at rt, filtered through a plug of Celite and the solvent was removed under vacuum. The residue was disolved in EtOAc, washed with NaHCO₃ sat solution, water and brine, dried over Na₂SO₄, filtered and concentrated. Purification of the residue by flash chromatography, silica gel, gradient DCM to 20% MeOH afforded the title compound (248 mg, 61% yield).

¹H-NMR, (CDCl₃, 400 MHz), δ (ppm): 7.44 (t, J=8.6 Hz, 1H), 6.86 (m, 2H), 4.13 (q, J=6.9 Hz, 2H), 3.77 (m, 2H), 3.43 (m, 2H), 2.92 (s, 3H), 2.69 (d, J=1.6 Hz, 3H), 1.50 (t, J=6.9 Hz, 3H), 1.42 (m, 9H).

### Ex 64: tert-Butyl 2-cyano-2-(2-(4-ethoxy-2-fluorophenyl)-3,4-dimethyl-2H-pyrazolo[3,4-d]pyridazin-7-yl)-3-phenylpropanoate

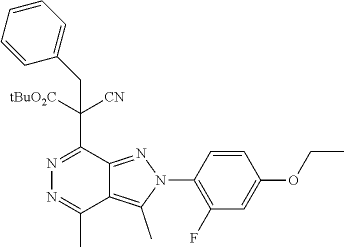 [SMILES: CCOc1ccc(-n2nc3c(C(C#N)(Cc4ccccc4)C(=O)OC(C)(C)C)nnc(C)c3c2C)c(F)c1]

To a suspension of NaH (60% in mineral oil, 7 mg, 0.176 mmol) in DMF (2 mL), a solution of tert-butyl 2-cyano-2-(2-(4-ethoxy-2-fluorophenyl)-3,4-dimethyl-2H-pyrazolo[3,4-d]pyridazin-7-yl)acetate (Ex 61, 50 mg, 0.118 mmol) in DMF (1 mL) was added at rt and stirred for 10 min. Benzyl bromide (24 mg, 0.141 mmol) was added and the mixture was stirred for 2 h. Water was added, extracted with EtOAc and the organic layer was concentrated. Purification of the residue by flash chromatography, silica gel, gradient hexane to EtOAc, afforded the title compound (21 mg, 34% yield).

HPLC (Method A): Ret, 8.83 min ; ESI⁺-MS m/z, 516.3 (M+H).

### Ex 65: 2-(2-(4-Ethoxy-2-fluorophenyl)-3,4-dimethyl-2H-pyrazolo[3,4-d]pyridazin-7-yl)-3-phenylpropanenitrile

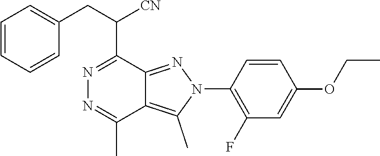 [SMILES: CCOc1ccc(-n2nc3c(C(C#N)Cc4ccccc4)nnc(C)c3c2C)c(F)c1]

tert-Butyl 2-cyano-2-(2-(4-ethoxy-2-fluorophenyl)-3,4-dimethyl-2H-pyrazolo[3,4-d]pyridazin-7-yl)-3-phenylpropanoate (Ex 64) was treated in the conditions described for Ex 62, at 120° C. to afford the title compound (89% yield).

HPLC (Method A): Ret, 8.84 min; ESI⁺-MS m/z, 416.2 (M+H).

Table of Examples with Binding to the μ-Opioid Receptor and the α₂δ-1 Subunit of the Voltage-Gated Calcium Channel:

Biological Activity

Pharmacological Study

Human α₂δ-1 Subunit of Caᵥ2.2 Calcium Channel Assay

Human α₂δ-1 enriched membranes (2.5 μg) were incubated with 15 nM of radiolabeled [3H]-Gabapentin in assay buffer containing Hepes-KOH 10 mM, pH 7.4. NSB (non specific binding) was measured by adding 10 μM pregabalin. The binding of the test compound was measured at five different concentrations. After 60 min incubation at 27° C., binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, pH 7.4. Filter plates were dried at 60° C. for 1 hour and 30 μl of scintillation cocktail were added to each well before radioactivity reading. Readings were performed in a Trilux 1450 Microbeta radioactive counter (Perkin Elmer).

Human μ-Opioid Receptor Radioligand Assay

To investigate binding properties of test compounds to human μ-opioid receptor, transfected CHO-K1 cell membranes and [³H]-DAMGO (Perkin Elmer, ES-542-C), as the radioligand, were used. The assay was carried out with 20 μg of membrane suspension, 1 nM of [³H]-DAMGO in either absence or presence of either buffer or 10 μM Naloxone for total and non-specific binding, respectively. Binding buffer contained Tris-HCl 50 mM, MgCl₂ 5 mM at pH 7.4. Plates were incubated at 27° C. for 60 minutes. After the incubation period, the reaction mix was then transferred to MultiScreen HTS, FC plates (Millipore), filtered and plates were washed 3 times with ice-cold 10 mM Tris-HCL (pH 7.4). Filters were dried and counted at approximately 40% efficiency in a MicroBeta scintillation counter (Perkin-Elmer) using EcoScint liquid scintillation cocktail. Preferably, transfected CHO-K1 cell membranes (20 μg) were incubated with 1 nM of [³H]-DAMGO in assay buffer containing Tris-HCl 50 mM, MgCl2 5 mM at pH 7.4. NBS (non-specific binding) was measured by adding 10 μM Naloxone. The binding of the test compound was measured at five different concentrations. Plates were incubated at 27° C. for 60 minutes. After the incubation period, the reaction mix was then transferred to MultiScreen HTS, FC plates (Millipore), filtered and plates were washed 3 times with ice-cold 10 mM Tris-HCL (pH 7.4). Filters were dried and counted at approximately 40% efficiency in a MicroBeta scintillation counter (Perkin-Elmer) using EcoScint liquid scintillation cocktail.

Results:

As this invention is aimed at providing a compound or a chemically related series of compounds which act as dual ligands of the α₂δ subunit of voltage-gated calcium channels and the p-opioid receptor it is a very preferred embodiment in which the compounds are selected which act as dual ligands of the α₂δ subunit of voltage-gated calcium channels and the μ-opioid receptor and especially compounds which have a binding expressed as Kᵢ responding to the following scales:

Kᵢ(μ) is preferably<1000 nM, more preferably<500 nM, even more preferably<100 nM.

Kᵢ(α₂δ-1) is preferably<10000 nM, more preferably<5000 nM, even more preferably<3000 nM or even more preferably<500 nM.

The following scale has been adopted for representing the binding to μ-opioid receptor expressed as Kᵢ:

+ Kᵢ(μ)>=500 nM

++ Kᵢ(μ)<500 nM

The following scale has been adopted for representing the binding to the α₂δ-1 subunit of voltage-gated calcium channels expressed as Kᵢ:

+ Kᵢ(α₂δ-1)>=5000 nM

++ 3000 nM<=Kᵢ(α₂δ-1)<5000 nM

+++ 500 nM<=Kᵢ(α₂δ-1)<3000 nM

++++ Kᵢ(α₂δ-1)<500 nM

All compounds prepared in the present application exhibit binding to the α₂δ-1 subunit of voltage-gated calcium channels and optionally additionally to the μ-opioid receptor, in particular the following binding results are shown:

| | α2δ1 | Mu |
|---|---|---|
| | binding | binding |
| Ex | activity | activity |
| 1 | + | + |
| 2 | + | + |
| 3 | + | + |
| 4 | + | + |
| 5 | + | + |
| 6 | +++ | + |
| 7 | +++ | + |
| 8 | +++ | + |
| 9 | +++ | + |
| 10 | ++ | + |
| 11 | + | + |
| 12 | + | + |
| 13 | + | + |
| 14 | +++ | + |
| 15 | + | + |
| 16 | + | + |
| 17 | + | + |
| 18 | +++ | + |
| 19 | + | + |
| 20 | +++ | + |
| 21 | + | + |
| 22 | + | + |
| 23 | + | + |
| 24 | ++ | + |
| 25 | +++ | ++ |
| 26 | ++++ | ++ |
| 27 | +++ | + |
| 28 | +++ | ++ |
| 29 | +++ | + |
| 30 | +++ | + |
| 31 | +++ | + |
| 32 | +++ | + |
| 33 | +++ | + |
| 34 | ++ | + |
| 35 | +++ | + |
| 36 | +++ | + |
| 37 | +++ | + |
| 38 | +++ | + |
| 39 | +++ | + |
| 40 | + | + |
| 41 | + | + |
| 42 | +++ | + |
| 43 | +++ | ++ |
| 44 | +++ | ++ |
| 45 | +++ | + |
| 46 | +++ | + |
| 47 | +++ | + |
| 48 | +++ | + |
| 49 | +++ | + |
| 50 | +++ | + |
| 51 | +++ | + |
| 52 | ++ | + |
| 53 | +++ | + |
| 54 | +++ | + |
| 55 | +++ | + |
| 56 | +++ | + |
| 57 | +++ | + |
| 58 | +++ | + |
| 59 | + | + |
| 60 | +++ | + |
| 61 | +++ | + |
| 62 | +++ | + |
| 63 | + | + |
| 64 | + | + |
| 65 | +++ | + |

1-13. (canceled)

14. A compound of general Formula (In): 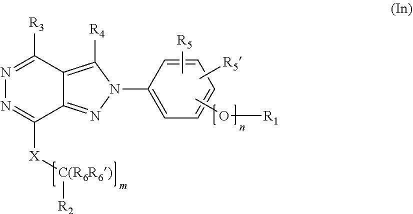
wherein
m is 0, 1, 2, 3, 4 or 5;
n is 0 or 1;
X is selected from the group consisting of a bond, —C(RₓR_{x′})—, —CH═CH—, —CH₂CH₂—, substituted or unsubstituted cycloalkyl, substituted or unsubstituted aryl and substituted or unsubstituted heterocyclyl; wherein
Rₓ is selected from the group consisting of hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;
R_{x′} is selected from the group consisting of hydrogen, substituted or unsubstituted alkylaryl, substituted or unsubstituted alkylcycloalkyl and substituted or unsubstituted alkylheterocyclyl;
wherein R₈ is selected from the group consisting of hydrogen, substituted or unsubstituted C₁₋₆ alkyl, substituted or unsubstituted C₂₋₆ alkenyl and substituted or unsubstituted C₂₋₆ alkynyl;
R₁ is selected from the group consisting of substituted or unsubstituted C₁₋₆ alkyl, substituted or unsubstituted C₂₋₆ alkenyl and substituted or unsubstituted C₂₋₆ alkynyl;
R₂ is selected from the group consisting of —NR₇R_{7′″}, —CN and substituted or unsubstituted N-containing-heterocyclyl;
wherein R₇ is selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc;
and wherein R_{7′″} is selected from the group consisting of hydrogen, substituted or unsubstituted C₁₋₆ alkyl, substituted or unsubstituted C₂₋₆ alkenyl and substituted or unsubstituted C₂₋₆ alkynyl;
R₃ is selected from the group consisting of hydrogen, substituted or unsubstituted C₁₋₆ alkyl, substituted or unsubstituted C₂₋₆ alkenyl and substituted or unsubstituted C₂₋₆ alkynyl;
R₄ is selected from the group consisting of hydrogen, substituted or unsubstituted C₁₋₆ alkyl, substituted or unsubstituted C₂₋₆ alkenyl and substituted or unsubstituted C₂₋₆ alkynyl;
R₅ and R_{5′} are independently selected from the group consisting of hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9″}, —SR₉, —S(O)R₉, S(O)₂R₉, —CN, haloalkyl, haloalkoxy, —C(O)OR₉, —C(O)NR₉R_{9′}, —NR₉S(O)₂NR_{9′}R_{9″}, and C(CH₃)₂—OR₉;
wherein R₉, R_{9′} and R_{9″} are independently selected from the group consisting of hydrogen, unsubstituted C₁₋₆ alkyl, unsubstituted C₂₋₆ alkenyl, and unsubstituted C₂₋₆ alkynyl;
and wherein R_{9′″} is selected from the group consisting of hydrogen, unsubstituted C₁₋₈ alkyl, unsubstituted C₂₋₈ alkenyl, unsubstituted C₂₋₈ alkynyl and -Boc;
R₆ and R_{6′} are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
optionally as a stereoisomer, including enantiomers and diastereomers, a racemate or as a mixture of at least two stereoisomers, including enantiomers and/or diastereomers, in any mixing ratio, or a corresponding salt thereof, or a corresponding solvate thereof;
with the following proviso applying:
—X—[CR₆R_{6′}]ₘ—R₂ is attached to the pyrazolopyridazine structure through a carbon atom;
and the following compounds are further excluded:

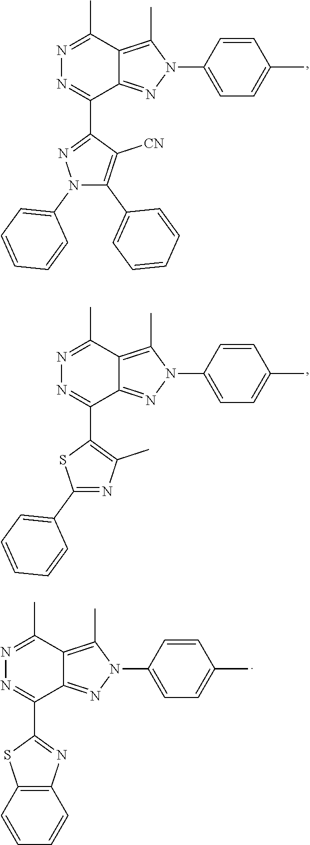

15. The compound according to claim 14, which is a compound of formula (I) 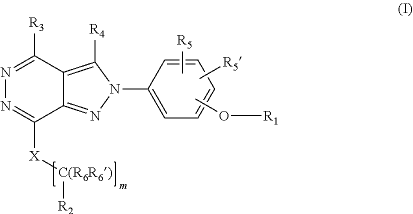
wherein
m is 0, 1, 2, 3, 4 or 5;
X is selected from the group consisting of a bond, —C(RₓR_{x′})—, —CH═CH—, —CH₂CH₂—, substituted or unsubstituted cycloalkyl, substituted or unsubstituted aryl and substituted or unsubstituted heterocyclyl; wherein
Rₓ is selected from the group consisting of hydrogen, substituted or unsubstituted C₁₋₆ alkyl, substituted or unsubstituted C₂₋₆ alkenyl, substituted or unsubstituted C₂₋₆ alkynyl, —C(O)R₈ and —C(O)OR₈;
R_{x′} is selected from the group consisting of hydrogen, substituted or unsubstituted alkylaryl, substituted or unsubstituted alkylcycloalkyl and substituted or unsubstituted alkylheterocyclyl;
wherein R₈ is selected from the group consisting of hydrogen, substituted or unsubstituted C₁₋₆ alkyl, substituted or unsubstituted C₂₋₆ alkenyl and substituted or unsubstituted C₂₋₆ alkynyl;
R₁ is selected from the group consisting of substituted or unsubstituted C₁₋₆ alkyl, substituted or unsubstituted C₂₋₆ alkenyl and substituted or unsubstituted C₂₋₆ alkynyl;
R₂ is selected from the group consisting of —NR₇R_{7′″}, —CN and substituted or unsubstituted N-containing-heterocyclyl;
wherein R₇ is selected from the group consisting of hydrogen, substituted or unsubstituted C₁₋₆ alkyl, substituted or unsubstituted C₂₋₆ alkenyl, substituted or unsubstituted C₂₋₆ alkynyl, substituted or unsubstituted alkylcycloalkyl, substituted or unsubstituted alkylaryl, substituted or unsubstituted alkylheterocyclyl and -Boc;
and wherein R_{7′″} is selected from the group consisting of hydrogen, substituted or unsubstituted C₁₋₆ alkyl, substituted or unsubstituted C₂₋₆ alkenyl and substituted or unsubstituted C₂₋₆ alkynyl;
R₃ is selected from the group consisting of hydrogen, substituted or unsubstituted C₁₋₆ alkyl, substituted or unsubstituted C₂₋₆ alkenyl and substituted or unsubstituted C₂₋₆ alkynyl;
R₄ is selected from the group consisting of hydrogen, substituted or unsubstituted C₁₋₆ alkyl, substituted or unsubstituted C₂₋₆ alkenyl and substituted or unsubstituted C₂₋₆ alkynyl;
R₅ and R_{5′} are independently selected from the group consisting of hydrogen, halogen, —R₉, —OR₉, —NO₂, —NR₉R_{9′″}, NR₉C(O)R_{9′}, —NR₉S(O)₂R_{9′}, —S(O)₂NR₉R_{9′}, —NR₉C(O)NR_{9′}R_{9″}, —SR₉ , —S(O)R₉, S(O)₂R₉, —CN, haloalkyl, haloalkoxy, —C(O)OR₉, —C(O)NR₉R_{9′}, —NR₉S(O)₂NR_{9′}R_{9″}, and C(CH₃)₂—OR₉;
wherein R₉, R_{9′} and R_{9″} are independently selected from the group consisting of hydrogen, unsubstituted C₁₋₆ alkyl, unsubstituted C₂₋₆ alkenyl, and unsubstituted C₂₋₆ alkynyl;
and wherein R_{9′″} is selected from the group consisting of hydrogen, unsubstituted C₁₋₈ alkyl, unsubstituted C₂₋₈ alkenyl, unsubstituted C₂₋₈ alkynyl and -Boc;
R₆ and R_{6′} are independently selected from the group consisting of hydrogen, substituted or unsubstituted C₁₋₆ alkyl, substituted or unsubstituted C₂₋₆ alkenyl and substituted or unsubstituted C₂₋₆ alkynyl;
optionally as a stereoisomer, including enantiomers and diastereomers, a racemate or as a mixture of at least two stereoisomers, including enantiomers and/or diastereomers, in any mixing ratio, or a corresponding salt thereof, or a corresponding solvate thereof;
with the following proviso applying:
—X—[CR₆R_{6′}]ₘ—R₂ is attached to the pyrazolopyridazine structure through a carbon atom.

16. The compound according to claim 14, which is a compound of formula (I′) 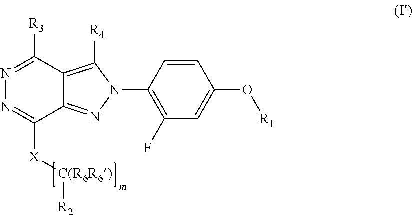
wherein R₁, R₂, R₃, R₄, R₆, R_{6′}, X and m are as defined in claim 14.

17. The compound according to claim 14, which is a compound of formula (I²′) 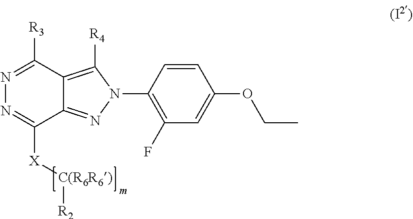
wherein R₂, R₃, R₄, R₆, R_{6′}, X and m are as defined in claim 14.

18. The compound according to claim 14, which is a compound of formula (I³′) 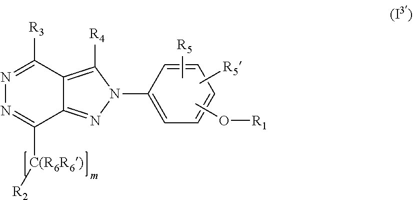
wherein R₁, R₂, R₃, R₄, R₅, R_{5′}, R₆, R_{6′} and m are as defined in claim 14.

19. The compound according to claim 14, which is a compound of formula (I⁴′) 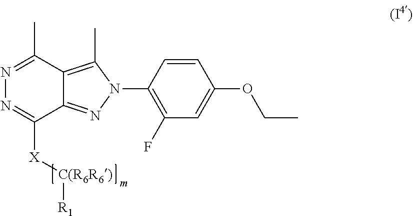
wherein R₂, R₆, R_{6′}, X and m are as defined in claim 14.

20. The compound according to claim 14, which is a compound of formula (I⁵′) 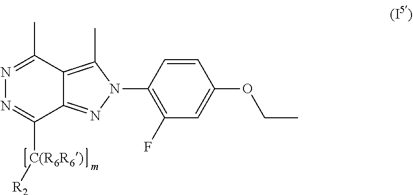
wherein R₂, R₆, R_{6′} and m are as defined in claim 14.

21. The compound according to claim 14, which is selected from the group consisting of:
tert-Butyl 4-(2-(4-ethoxy-2-fluorophenyl)-3,4-dimethyl-2H-pyrazolo[3,4-d]pyridazin-7-yl)-3,6-dihydropyridine-1(2H)-carboxylate,
2-(4-Ethoxy-2-fluorophenyl)-3,4-dimethyl-7-(1-methyl-1,2,3,6-tetrahydropyridin-4-yl)-2H-pyrazolo[3,4-d]pyridazine,
tert-Butyl 5-(2-(4-ethoxy-2-fluorophenyl)-3,4-dimethyl-2H-pyrazolo[3,4-d]pyridazin-7-yl)-3,4-dihydropyridine-1(2H)-carboxylate,
tert-Butyl (E)-(3-(2-(4-ethoxy-2-fluorophenyl)-3,4-dimethyl-2H-pyrazolo[3,4-d]pyridazin-7-yl)ally)carbamate,
tert-Butyl (E)-(3-(2-(4-ethoxy-2-fluorophenyl)-3,4-dimethyl-2H-pyrazolo[3,4-d]pyridazin-7-yl)allyl)(methyl)carbamate,
1-(4-(2-(4-Ethoxy-2-fluorophenyl)-3,4-dimethyl-2H-pyrazolo[3,4-d]pyridazin-7-yl)phenyl)-N,N-dimethylmethanamine,
2-(4-(2-(4-Ethoxy-2-fluorophenyl)-3,4-dimethyl-2H-pyrazolo[3,4-d]pyridazin-7-yl)phenyl)-N,N-dimethylethan-1-amine,
1-(3-(2-(4-Ethoxy-2-fluorophenyl)-3,4-dimethyl-2H-pyrazolo[3,4-d]pyridazin-7-yl)phenyl)-N,N-dimethylmethanamine,
2-(3-(2-(4-Ethoxy-2-fluorophenyl)-3,4-dimethyl-2H-pyrazolo[3,4-d]pyridazin-7-yl)phenyl)-N,N-dimethylethan-1-amine,
2-(6-(2-(4-Ethoxy-2-fluorophenyl)-3,4-dimethyl-2H-pyrazolo[3,4-d]pyridazin-7-yl)-1H-benzo[d]imidazol-1-yl)-N,N- dimethylethan-1-amine,
tert-butyl (2-(6-(2-(4-ethoxy-2-fluorophenyl)-3,4-dimethyl-2H-pyrazolo[3,4-d]pyridazin-7-yl)-1H-benzo[d]imidazol-1-yl)ethyl)carbamate,
tert-Butyl 3-(2-(4-ethoxy-2-fluorophenyl)-3,4-dimethyl-2H-pyrazolo[3,4-d]pyridazin-7-yl)azetidine-1-carboxylate,
tert-Butyl 4-(2-(4-ethoxy-2-fluorophenyl)-3,4-dimethyl-2H-pyrazolo[3,4-d]pyridazin-7-yl)piperidine-1-carboxylate,
2-(4-Ethoxy-2-fluorophenyl)-3,4-dimethyl-7-(1-methylpiperidin-4-yl)-2H-pyrazolo[3,4-d]pyridazine,
tert-Butyl-3-(2-(4-ethoxy-2-fluorophenyl)-3,4-dimethyl-2H-pyrazolo[3,4-d]pyridazin-7-yl)piperidine-1-carboxylate,
tert-Butyl-(3-(2-(4-ethoxy-2-fluorophenyl)-3,4-dimethyl-2H-pyrazolo[3,4-d]pyridazin-7-yl)propyl)carbamate,
tert-Butyl-(3-(2-(4-ethoxy-2-fluorophenyl)-3,4-dimethyl-2H-pyrazolo[3,4-d]pyridazin-7-yl)propyl)(methyl)carbamate,
2-(4-Ethoxy-2-fluorophenyl)-3,4-dimethyl-7-(piperidin-4-yl)-2H-pyrazolo[3,4-d]pyridazine,
2-(4-Ethoxy-2-fluorophenyl)-3,4-dimethyl-7-(piperidin-3-yl)-2H-pyrazolo[3,4-d]pyridazine,
3-(2-(4-Ethoxy-2-fluorophenyl)-3,4-dimethyl-2H-pyrazolo[3,4-d]pyridazin-7-yl)propan-1-amine,
3-(2-(4-Ethoxy-2-fluorophenyl)-3,4-dimethyl-2H-pyrazolo[3,4-d]pyridazin-7-yl)-N-methylpropan-1-amine,
7-(Azetidin-3-yl)-2-(4-ethoxy-2-fluorophenyl)-3,4-dimethyl-2H-pyrazolo[3,4-d]pyridazine,
2-(2-(4-Ethoxy-2-fluorophenyl)-3,4-dimethyl-2H-pyrazolo[3,4-d]pyridazin-7-yl)ethan-1-amine,
2-(6-(2-(4-Ethoxy-2-fluorophenyl)-3,4-dimethyl-2H-pyrazolo[3,4-d]pyridazin-7-yl)-1H-benzo[d]imidazol-1-yl)ethan-1-amine,
2-(4-Ethoxy-2-fluorophenyl)-3,4-dimethyl-7-(1-phenethylpiperidin-4-yl)-2H-pyrazolo[3,4-d]pyridazine,
7-(1-Benzylpiperidin-4-yl)-2-(4-ethoxy-2-fluorophenyl)-3,4-dimethyl-2H-pyrazolo[3,4-d]pyridazine,
2-(4-Ethoxy-2-fluorophenyl)-7-(1-(4-fluorobenzyl)piperidin-4-yl)-3,4-dimethyl-2H-pyrazolo[3,4-d]pyridazine,
2-(4-Ethoxy-2-fluorophenyl)-3,4-dimethyl-7-(1-(pyridin-2-ylmethyl)piperidin-4-yl)-2H-pyrazolo[3,4-d]pyridazine,
2-(4-Ethoxy-2-fluorophenyl)-3,4-dimethyl-7-(1-(pyridin-3-ylmethyl)piperidin-4-yl)-2H-pyrazolo[3,4-d]pyridazine,
2-(4-Ethoxy-2-fluorophenyl)-3,4-dimethyl-7-(1-(pyridin-4-ylmethyl)piperidin-4-yl)-2H-pyrazolo[3,4-d]pyridazine,
2-((4-(2-(4-Ethoxy-2-fluorophenyl)-3,4-dimethyl-2H-pyrazolo[3,4-d]pyridazin-7-yl)piperidin-1-ylmethyl)pyridin-3-ol,
7-(1-(Cyclopropyl-methyl)piperidin-4-yl)-2-(4-ethoxy-2-fluorophenyl)-3,4-dimethyl-2H-pyrazolo[3,4-d]pyridazine,
2-(4-Ethoxy-2-fluorophenyl)-3,4-dimethyl-7-(1-((tetrahydro-2H-pyran-4-yl)methyl)piperidin-4-yl)-2H-pyrazolo[3,4-d]pyridazine,
2-(4-Ethoxy-2-fluorophenyl)-3,4-dimethyl-7-(1-(2-(tetrahydro-2H-pyran-4-yl)ethyl)piperidin-4-yl)-2H-pyrazolo[3,4-d]pyridazine,
2-(4-Ethoxy-2-fluorophenyl)-3,4-dimethyl-7-(1-((1-(pyridin-2-ylmethyl)-1H-1,2,3-triazol-4-yl)methyl)piperidin-4-yl)-2H-pyrazolo[3,4-d]pyridazine,
7-(1-Benzylpiperidin-3-yl)-2-(4-ethoxy-2-fluorophenyl)-3,4-dimethyl-2H-pyrazolo[3,4-d]pyridazine,
2-(4-Ethoxy-2-fluorophenyl)-3,4-dimethyl-7-(1-phenethylazetidin-3-yl)-2H-pyrazolo[3,4-d]pyridazine,
N-Benzyl-3-(2-(4-ethoxy-2-fluorophenyl)-3,4-dimethyl-2H-pyrazolo[3,4-d]pyridazin-7-yl)propan-1-amine,
3-(2-(4-Ethoxy-2-fluorophenyl)-3,4-dimethyl-2H-pyrazolo[3,4-d]pyridazin-7-yl)-N-methyl-N-phenethylpropan-1-amine,
N-Benzyl-2-(2-(4-ethoxy-2-fluorophenyl)-3,4-dimethyl-2H-pyrazolo[3,4-d]pyridazin-7-yl)ethan-1-amine,
2-(2-(4-Ethoxy-2-fluorophenyl)-3,4-dimethyl-2H-pyrazolo[3,4-d]pyridazin-7-yl)-N-phenethylethan-1-amine,
2-(4-Ethoxy-2-fluorophenyl)-3,4-dimethyl-7-(1-(2-(pyridin-2-yl)ethyl)piperidin-4-yl)-2H-pyrazolo[3,4-d]pyridazine,
2-(4-Ethoxy-2-fluorophenyl)-7-(1-(2-fluorophenethyl)piperidin-4-yl)-3,4-dimethyl-2H-pyrazolo[3,4-d]pyridazine,
2-(4-Ethoxy-2-fluorophenyl)-7-(1-(3-fluorophenethyl)piperidin-4-yl)-3,4-dimethyl-2H-pyrazolo[3,4-d]pyridazine,
2-(4-Ethoxy-2-fluorophenyl)-7-(1-(3-methoxyphenethyl)piperidin-4-yl)-3,4-dimethyl-2H-pyrazolo[3,4-d]pyridazine,
2-(4-Ethoxy-2-fluorophenyl)-7-(1-(4-fluorophenethyl)piperidin-4-yl)-3,4-dimethyl-2H-pyrazolo[3,4-d]pyridazine,
2-(4-Ethoxy-2-fluorophenyl)-3,4-dimethyl-7-(1-(2-(pyridin-3-yl)ethyl)piperidin-4-yl)-2H-pyrazolo[3,4-d]pyridazine,
2-(4-Ethoxy-2-fluorophenyl)-7-(1-((3-fluoropyridin-2-yl)methyl)piperidin-4-yl)-3,4-dimethyl-2H-pyrazolo[3,4-d]pyridazine,
2-(4-Ethoxy-2-fluorophenyl)-3,4-dimethyl-7-(1-((3-(trifluoromethyl)pyridin-2-yl)methyl)piperidin-4-yl)-2H-pyrazolo[3,4-d]pyridazine,
2-(4-Ethoxy-2-fluorophenyl)-7-(1-((3-methoxypyridin-2-yl)methyl)piperidin-4-yl)-3,4-dimethyl-2H-pyrazolo[3,4-d]pyridazine,
7-(1-((3-Chloropyridin-2-yl)methyl)piperidin-4-yl)-2-(4-ethoxy-2-fluorophenyl)-3,4-dimethyl-2H-pyrazolo[3,4-d]pyridazine,
2-(4-Ethoxy-2-fluorophenyl)-3,4-dimethyl-7-(1-((4-(trifluoromethyl)pyridin-2-yl)methyl)piperidin-4-yl)-2H-pyrazolo[3,4-d]pyridazine,
7-(1-((5-Chloropyridin-2-yl)methyl)piperidin-4-yl)-2-(4-ethoxy-2-fluorophenyl)-3,4-dimethyl-2H-pyrazolo[3,4-d]pyridazine,
6-((4-(2-(4-Ethoxy-2-fluorophenyl)-3,4-dimethyl-2H-pyrazolo[3,4-d]pyridazin-7-yl)piperidin-1-yl)methyl)pyridin-3-ol,
2-(4-Ethoxy-2-fluorophenyl)-7-(1-((5-fluoropyridin-2-yl)methyl)piperidin-4-yl)-3,4-dimethyl-2H-pyrazolo[3,4-d]pyridazine,
2-(4-Ethoxy-2-fluorophenyl)-3,4-dimethyl-7-(1-((5-(trifluoromethyl)pyridin-2-yl)methyl)piperidin-4-yl)-2H-pyrazolo[3,4-d]pyridazine,
2-(4-Ethoxy-2-fluorophenyl)-3,4-dimethyl-7-(1-(2-(pyridin-4-yl)ethyl)piperidin-4-yl)-2H-pyrazolo[3,4-d]pyridazine,
2-(4-(2-(4-Ethoxy-2-fluorophenyl)-3,4-dimethyl-2H-pyrazolo[3,4-d]pyridazin-7-yl)piperidin-1-yl)-1-phenylethan-1-ol,
2-(4-Ethoxy-2-fluorophenyl)-3,4-dimethyl-2H-pyrazolo[3,4-d]pyridazine-7-carbonitrile,
3-(1-((2-(4-Ethoxy-2-fluorophenyl)-3,4-dimethyl-2H-pyrazolo[3,4-d]pyridazin-7-yl)methyl)piperidin-4-yl)phenol,
tert-Butyl 2-cyano-2-(2-(4-ethoxy-2-fluorophenyl)-3,4-dimethyl-2H-pyrazolo[3,4-d]pyridazin-7-yl)acetate,
2-(2-(4-Ethoxy-2-fluorophenyl)-3,4-dimethyl-2H-pyrazolo[3,4-d]pyridazin-7-yl)acetonitrile,
tert-Butyl (2-(2-(4-ethoxy-2-fluorophenyl)-3,4-dimethyl-2H-pyrazolo[3,4-d]pyridazin-7-yl)ethyl)carbamate,
tert-Butyl 2-cyano-2-(2-(4-ethoxy-2-fluorophenyl)-3,4-dimethyl-2H-pyrazolo[3,4-d]pyridazin-7-yl)-3-phenylpropanoate, and
2-(2-(4-Ethoxy-2-fluorophenyl)-3,4-dimethyl-2H-pyrazolo[3,4-d]pyridazin-7-yl)-3-phenylpropanenitrile.

22. A process for the preparation of the compound according to claim 14, wherein when the compound is
a) a compound of Formula (Ia) wherein X is —CH═CH— 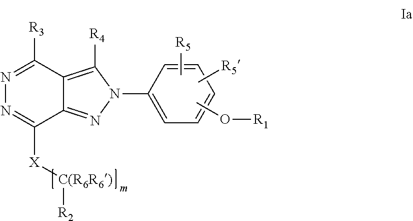
said process comprises the reaction of a compound of general formula II, wherein Y is an halogen, including chlorine, 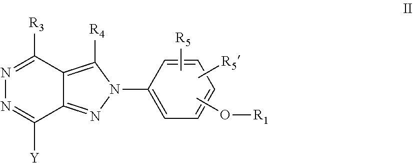
with a boronic acid (Z═H) or boronic ester (Z=Alkyl) of general formula III 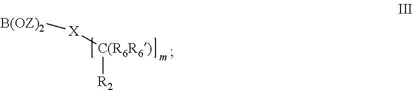
or
b) a compound of Formula (Ib) wherein X is —CH₂CH₂—, 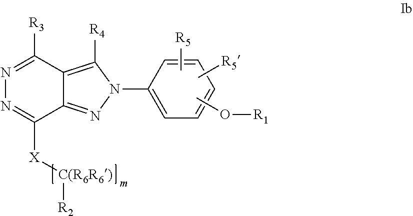
said process comprises the reduction of compounds of formula Ia, wherein X is —CH═CH— 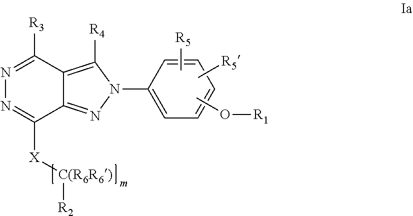
using suitable reductive reagents, preferably hydrogen in the presence of a catalyst, including Pd(OH)₂ on carbon, in an organic solvent, including MeOH; or
c) a compound of Formula (Ib), wherein X is —CH₂CH₂— 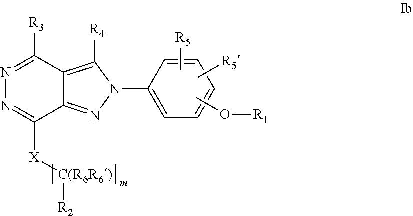
said process comprises the reaction of a compound of formula II with an organometalic reagent, prepared from a compound of general formula IV, 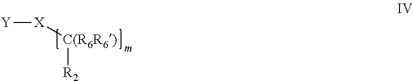
with a metal agent, including Zn; or
d) a compound of Formula (Ic), 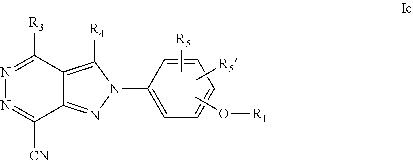
said process comprises the reaction of a compound of formula II, 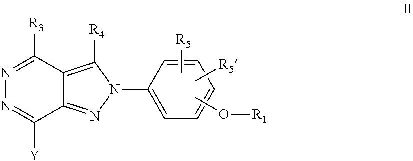
wherein Y is an halogen, including chlorine, with a cyanation reagent, including zinc cyanide, in the presence of a Pd catalyst; or
e) is a compound of Formula (Id), 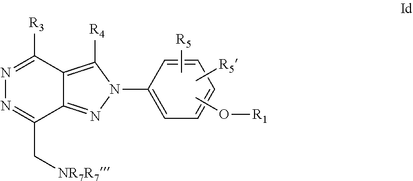
said process comprises treating a compound of formula Ic 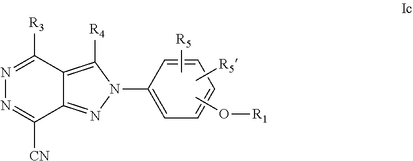
with an acid, including HCl, followed by a reduction reaction and final reductive amination with an amine of formula V,
NHR₇R_{7′″} V
or
f) a compound of Formula (Ie), 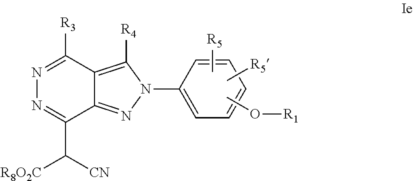
said process comprises the reaction of a compound of formula II, 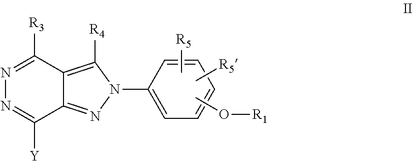
wherein Y is an halogen, including chlorine, with a reagent of formula VI, 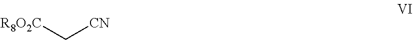
in the presence of a base; or
g) a compound of Formula (If), 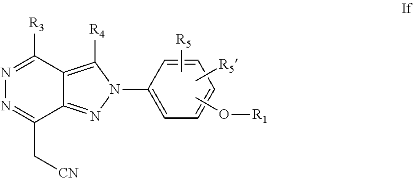
said process comprises the transformation of a compound of formula Ie, 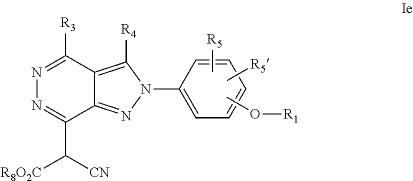
by heating at a suitable temperature, including in the range of 50-180° C., in an organic solvent; or
h) a compound of Formula (Ig), 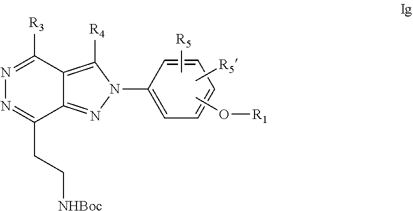
said process comprises the reduction of a compound of formula If 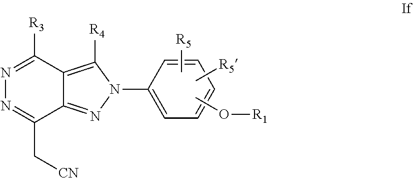
with a suitable reductive reagent, including sodium borohydride in the presence of NiCl₂.6H₂O and ditert-butyl dicarbonate, in an organic solvent; or
i) a compound of Formula (Ih), 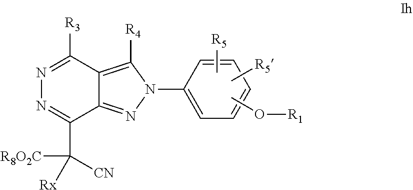
said process comprises the alkylation reaction of a compound of formula Ie 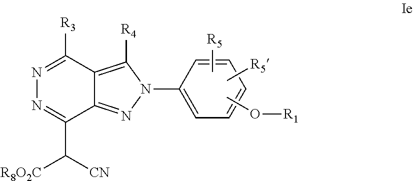
with a reagent of general formula VII
RxY VII
wherein Y is a good leaving group, including a halogen and sulfonate, in the presence of a base, including NaH, in an organic solvent; or
j) a compound of Formula (Ii), 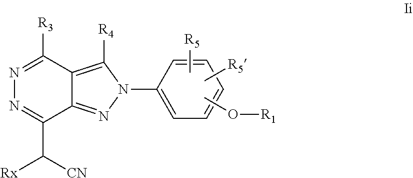
said process comprises the transformation of a compound of formula Ih, 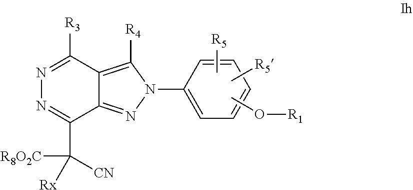
by heating at a suitable temperature, including in the range of 50-180° C., in an organic solvent, including hexafluoro-2-isopropanol, alternatively under microwave irradiation, wherein, unless otherwise defined, R₁, R₂, R₃, R₄, R₅, R_{5′}, R₆, R_{6′}, R₇, R_{7′″}, R₈, Rₓ, and m are as defined in claim 14.

23. A process for the preparation of the compound according to claim 14, employing a compound of Formula Ia, Ib, Ic, Id, Ie, If, Ig, Ih, Ii, II, III, IV, V, VI or VII: 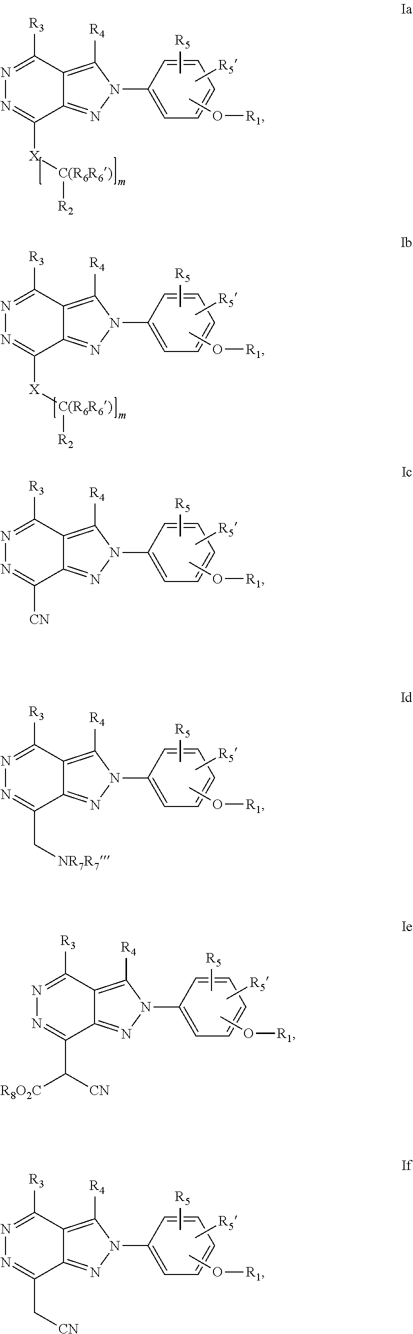 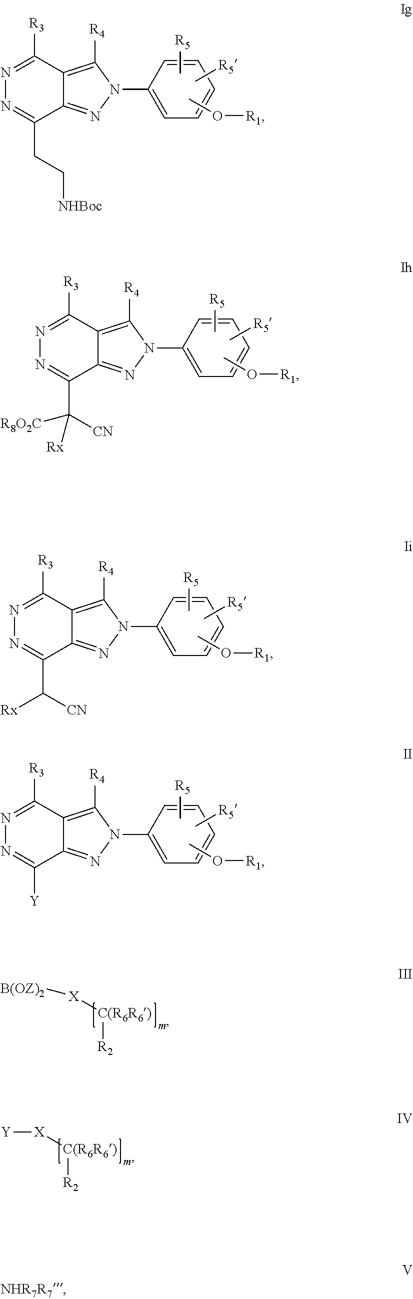 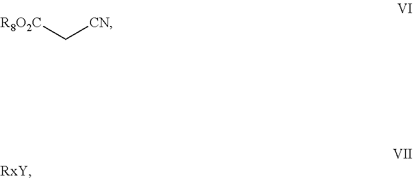
wherein Y is a halogen or a leaving group, Z is H or alkyl, and R₁, R₂, R₃, R₄, R₅, R_{5′}, R₆, R_{6′}, R₇, R_{7′″}, R₈, Rₓ, X and m are as defined in claim 14.

24. A pharmaceutical composition which comprises the compound according to claim 14, or a pharmaceutically acceptable salt thereof, and a pharmaceutically acceptable carrier, adjuvant or vehicle.

25. A method of treating pain in a subject in need thereof, comprising administration of an effective amount of the compound according to claim 14.

26. The method according to claim 14, wherein the pain is selected from the group consisting of medium to severe pain, visceral pain, chronic pain, cancer pain, migraine, inflammatory pain, acute pain or neuropathic pain, allodynia, and hyperalgesia.
